(19) United States
(12) Patent Application Publication
(10) Pub. No.: US 2022/0307033 A1
(43) Pub. Date: Sep. 29, 2022

(54) Treatment Of Liver Disease With Ring Finger Protein 213 (RNF213) Inhibitors

(71) Applicant: Regeneron Pharmaceuticals, Inc., Tarrytown, NY (US)

(72) Inventors: Luca Andrea Lotta, Tarrytown, NY (US); Niek Verweij, Tarrytown, NY (US); Manuel Allen Revez Ferreira, Tarrytown, NY (US); Aris Baras, Tarrytown, NY (US)

(21) Appl. No.: 17/683,468

(22) Filed: Mar. 1, 2022

Related U.S. Application Data
(60) Provisional application No. 63/155,327, filed on Mar. 2, 2021.
(60) Provisional application No. 63/159,027, filed on Mar. 10, 2021.

(51) Int. Cl. C12N 15/113; A61K 31/7088; A61K 38/46; C12Q 1/6883; A61P 1/16; C12N 15/11

(52) U.S. Cl. CPC C12N 15/1137; A61K 31/7088; A61K 38/465; C12Q 1/6883; A61P 1/16; C12N 15/111; C12Q 2600/106; C12Q 2600/158

(57) ABSTRACT

The present disclosure provides methods of treating subjects having a liver disease, and methods of identifying subjects having an increased risk of developing a liver disease.

### REFERENCE TO SEQUENCE LISTING

This application includes a Sequence Listing submitted electronically as a text file named 18923805901SEQ, created on Feb. 28, 2022, with a size of 13,777 kilobytes. The Sequence Listing is incorporated herein by reference.

### FIELD

The present disclosure relates generally to the treatment of subjects having a liver disease with Ring Finger Protein 213 (RNF213) inhibitors, and methods of identifying subjects having an increased risk of developing a liver disease.

### BACKGROUND

Chronic liver disease and cirrhosis are leading causes of morbidity and mortality in the United States accounting for 38,170 deaths (1.5% of total deaths) in 2014 (Kochanek et al., Nat'l. Vital Stat. Rep., 2016, 65, 1-122). The most common etiologies of cirrhosis in the U.S. are alcoholic liver disease, chronic hepatitis C, and nonalcoholic fatty liver disease (NAFLD), together accounting for about 80% of subjects awaiting liver transplant between 2004 and 2013 (Wong et al., Gastroenterology, 2015, 148, 547-555). The estimated prevalence of NAFLD in the U.S. is between 19 and 46 percent (Browning et al., Hepatology, 2004, 40, 1387-1395; Lazo et al., Am. J. Epidemiol., 2013, 178, 38-45; and Williams et al., Gastroenterology, 2011, 140, 124-131) and is rising over time (Younossi et al., Clin. Gastroenterol. Hepatol., 2011, 9, 524-530), likely in conjunction with increased rates of obesity, its primary risk factor (Cohen et al., Science, 2011, 332, 1519-1523). While significant advances have been made in the treatment of hepatitis C, there are currently no evidence-based treatments for alcoholic or nonalcoholic liver disease and cirrhosis.

Ring Finger Protein 213 (RNF213) is protein containing a C3HC4-type RING finger domain, which is a specialized type of Zn-finger that binds two atoms of zinc and is thought to be involved in mediating protein-protein interactions. The protein also contains an AAA domain, which is associated with ATPase activity. RNF213 is also known as E3 ubiquitin-protein ligase and is involved in angiogenesis and in the non-canonical Wnt signaling pathway in vascular development, where it acts by mediating ubiquitination and degradation of FLNA and NFATC2 downstream of RSPO3, leading to inhibition of the non-canonical Wnt signaling pathway and promoting vessel regression. In addition, RNF213 is a susceptibility gene for Moyamoya disease (MMD), a cerebrovascular disorder characterized by arterial occlusions and abnormal blood vessel generation. In addition, RNF213 plays a role in lipid metabolism modulating lipotoxicity, fat storage, and lipid droplet formation. RNF213^{-/-} MGI mouse show decreased body weight and leptin, increased food intake, increased glucose, and impaired insulin.

### SUMMARY

The present disclosure provides methods of treating a subject having liver disease, the methods comprising administering an RNF213 inhibitor to the subject.

The present disclosure also provides methods of treating a subject having fatty liver disease, the methods comprising administering an RNF213 inhibitor to the subject.

The present disclosure also provides methods of treating a subject having hepatocellular carcinoma, the methods comprising administering an RNF213 inhibitor to the subject.

The present disclosure also provides methods of treating a subject having liver cirrhosis, the methods comprising administering an RNF213 inhibitor to the subject.

The present disclosure also provides methods of treating a subject having liver fibrosis, the methods comprising administering an RNF213 inhibitor to the subject.

The present disclosure also provides methods of treating a subject having simple steatosis, steatohepatitis, or non-alcoholic steatohepatitis (NASH), the methods comprising administering an RNF213 inhibitor to the subject.

The present disclosure also provides methods of treating a subject with a therapeutic agent that treats or inhibits a liver disease, wherein the subject is suffering from a liver disease, the methods comprising the steps of: determining whether the subject has an RNF213 predicted loss-of-function or missense variant nucleic acid molecule encoding a human RNF213 polypeptide by: obtaining or having obtained a biological sample from the subject; and performing or having performed a genotyping assay on the biological sample to determine if the subject has a genotype comprising the RNF213 predicted loss-of-function or missense variant nucleic acid molecule; and when the subject is RNF213 reference, then administering or continuing to administer to the subject the therapeutic agent that treats or inhibits a liver disease in a standard dosage amount, and administering to the subject an RNF213 inhibitor; and when the subject is heterozygous for an RNF213 predicted loss-of-function or missense variant, then administering or continuing to administer to the subject the therapeutic agent that treats or inhibits a liver disease in an amount that is the same as or lower than a standard dosage amount, and administering to the subject an RNF213 inhibitor; wherein the presence of a genotype having the RNF213 predicted loss-of-function or missense variant nucleic acid molecule encoding the human RNF213 polypeptide indicates the subject has a reduced risk of developing a liver disease.

The present disclosure also provides methods of identifying a subject having an increased risk for developing a liver disease, wherein the methods comprise: determining or having determined the presence or absence of an RNF213 predicted loss-of-function or missense variant nucleic acid molecule encoding a human RNF213 polypeptide in a biological sample obtained from the subject; wherein: when the subject is RNF213 reference, then the subject has an increased risk for developing a liver disease; and when the subject is heterozygous for an RNF213 predicted loss-of-function or missense variant or homozygous for an RNF213 predicted loss-of-function or missense variant, then the subject has a decreased risk for developing a liver disease.

The present disclosure also provides therapeutic agents that treat or inhibit a liver disease for use in the treatment of a liver disease in a subject having: a genomic nucleic acid molecule having a nucleotide sequence encoding an RNF213 polypeptide, wherein the nucleotide sequence comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; an mRNA molecule having a nucleotide sequence encoding a human RNF213 polypeptide, wherein the nucleotide sequence comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; or a cDNA molecule having a nucleotide sequence encoding a human RNF213 polypeptide, wherein the nucleotide sequence comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof.

The present disclosure also provides RNF213 inhibitors for use in the treatment of a liver disease in a subject having: a genomic nucleic acid molecule having a nucleotide sequence encoding a human Ring Finger Protein 213 polypeptide, wherein the nucleotide sequence comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; an mRNA molecule having a nucleotide sequence encoding a human RNF213 polypeptide, wherein the nucleotide sequence comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; or a cDNA molecule having a nucleotide sequence encoding a human RNF213 polypeptide, wherein the nucleotide sequence comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

A rare variant in the RNF213 gene associated with a decreased risk of developing a liver disease in subjects has been identified in accordance with the present disclosure. For example, a genetic alteration that changes the adenine nucleotide of position 102,917 in the human RNF213 reference (see, SEQ ID NO:1) to guanine, or the guanine nucleotide of position 102,391 in the human RNF213 reference (see, SEQ ID NO:1) to cytosine, or the cytosine nucleotide of position 103,226 in the human RNF213 reference (see, SEQ ID NO:1) to thymine has been observed to indicate that the human having such an alteration may have a decreased risk of developing a liver disease. It is believed that no variants of the RNF213 gene or protein have any known association with a liver disease. Altogether, the genetic analyses described herein surprisingly indicate that the RNF213 gene and, in particular, variants in the RNF213 gene, associates with a decreased risk of developing a liver disease. Therefore, subjects that are RNF213 reference that have an increased risk of developing liver disease (such as, for example, fatty liver disease (including alcoholic fatty liver disease (AFLD) and NAFLD), hepatocellular carcinoma, liver cirrhosis, liver fibrosis, simple steatosis, steatohepatitis, non-alcoholic steatohepatitis (NASH), and parenchymal liver disease) may be treated such that liver disease is prevented, the symptoms thereof are reduced, and/or development of symptoms is repressed. Accordingly, the present disclosure provides methods of leveraging the identification of such variants in subjects to identify or stratify risk in such subjects of developing liver disease, or to diagnose subjects as having an increased risk of developing liver disease, such that subjects at risk or subjects with active disease may be treated accordingly.

It has been further observed in accordance with the present disclosure that an aggregate burden of certain variations in RNF213 associate with a lower risk of developing liver disease (such as, for example, fatty liver disease (including AFLD and NAFLD), hepatocellular carcinoma, liver cirrhosis, liver fibrosis, simple steatosis, steatohepatitis, non-alcoholic steatohepatitis (NASH), and parenchymal liver disease). Therefore, it is believed that humans having liver disease may be treated with molecules that inhibit RNF213. Accordingly, the present disclosure provides methods for leveraging the identification of such variants, and an aggregation burden of having such variants, in subjects to identify or stratify risk in such subjects of liver disease, or to diagnose subjects as having liver disease, such that subjects at risk or subjects with active disease may be treated.

For purposes of the present disclosure, any particular human can be categorized as having one of three RNF213 genotypes: i) RNF213 reference; ii) heterozygous for an RNF213 predicted loss-of-function or missense variant; or iii) homozygous for an RNF213 predicted loss-of-function or missense variant. A human is RNF213 reference when the human does not have a copy of an RNF213 predicted loss-of-function or missense variant nucleic acid molecule. A human is heterozygous for an RNF213 predicted loss-of-function or missense variant when the human has a single copy of an RNF213 predicted loss-of-function or missense variant nucleic acid molecule. As used herein, an RNF213 predicted loss-of-function variant nucleic acid molecule is any RNF213 nucleic acid molecule (such as, a genomic nucleic acid molecule, an mRNA molecule, or a cDNA molecule) encoding an RNF213 polypeptide having a partial loss-of-function, a complete loss-of-function, a predicted partial loss-of-function, or a predicted complete loss-of-function. A human who has an RNF213 polypeptide having a partial loss-of-function (or predicted partial loss-of-function) is hypomorphic for RNF213. The RNF213 predicted loss-of-function or missense variant nucleic acid molecule can be any nucleic acid molecule encoding RNF213 Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu. In some embodiments, the RNF213 predicted loss-of-function or missense variant nucleic acid molecule encodes RNF213 Glu3915Gly or Val3838Leu. A human is homozygous for an RNF213 predicted loss-of-function or missense variant when the human has two copies of an RNF213 predicted loss-of-function or missense variant nucleic acid molecule.

For subjects that are genotyped or determined to be RNF213 reference, such subjects have an increased risk of developing a liver disease (such as, for example, fatty liver disease (including AFLD and NAFLD), hepatocellular carcinoma, liver cirrhosis, liver fibrosis, simple steatosis, steatohepatitis, non-alcoholic steatohepatitis (NASH), and parenchymal liver disease). For subjects that are genotyped or determined to be either RNF213 reference or heterozygous for an RNF213 predicted loss-of-function or missense variant, such subjects can be treated with an RNF213 inhibitor.

In any of the embodiments described herein, the RNF213 predicted loss-of-function or missense variant nucleic acid molecule can be any RNF213 nucleic acid molecule (such as, for example, genomic nucleic acid molecule, mRNA molecule, or cDNA molecule) encoding an RNF213 polypeptide having a partial loss-of-function, a complete loss-of-function, a predicted partial loss-of-function, or a predicted complete loss-of-function. For example, the RNF213 predicted loss-of-function or missense variant nucleic acid molecule can be any nucleic acid molecule encoding RNF213 Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu. In some embodiments, the RNF213 predicted loss-of-function or missense variant nucleic acid molecule encodes RNF213 Glu3915Gly or Val3838Leu.

In any of the embodiments described herein, the RNF213 polypeptide can be any RNF213 polypeptide having a partial loss-of-function, a complete loss-of-function, a predicted partial loss-of-function, or a predicted complete loss-of-function. In any of the embodiments described herein, the RNF213 polypeptide can be any of the RNF213 polypeptides described herein including, for example, RNF213 Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu. In some embodiments, the RNF213 polypeptide is RNF213 Glu3915Gly or Val3838Leu.

In any of the embodiments described herein, the liver disease is a fatty liver disease, including AFLD and NAFLD, hepatocellular carcinoma, liver cirrhosis, liver fibrosis, simple steatosis, steatohepatitis, NASH, or parenchymal liver disease. In some embodiments, the liver disease is a fatty liver disease. In some embodiments, the liver disease is AFLD. In some embodiments, the liver disease is NAFLD. In some embodiments, the liver disease is hepatocellular carcinoma. In some embodiments, the liver disease is liver cirrhosis. In some embodiments, the liver disease is liver fibrosis. In some embodiments, the liver disease is simple steatosis. In some embodiments, the liver disease is steatohepatitis. In some embodiments, the liver disease is NASH. In some embodiments, the liver disease is parenchymal liver disease.

In some embodiments, the liver disease is liver damage, deposition of liver fat, liver inflammation, toxic liver disease, immune liver disease, or elevated alanine aminotransferase (ALT). In some embodiments, the liver disease is liver damage. In some embodiments, the liver damage is measured by elevation of liver enzymes. In some embodiments, the liver disease is deposition of liver fat. In some embodiments, the deposition of liver fat is identified by imaging, biopsy, or other procedure. In some embodiments, the liver disease is liver inflammation. In some embodiments, the liver inflammation is identified by biopsy, imaging, or other procedure. In some embodiments, the liver disease is toxic liver disease. In some embodiments, the liver disease is immune liver disease. In some embodiments, the liver disease is elevated ALT. In some embodiments, the liver disease is due to accumulation of metals, proteinaceous material, bile acids, or other irritant or pro-inflammatory materials. In some embodiments, the liver disease is due to accumulation of metals, such as iron. In some embodiments, the liver disease is due to accumulation of proteinaceous material, such as in alpha 1 antitrypsin deficiency. In some embodiments, the liver disease is due to accumulation of bile acids. In some embodiments, the liver disease is due to accumulation of an irritant. In some embodiments, the liver disease is due to accumulation of a pro-inflammatory material.

Symptoms of liver disease include, but are not limited to, enlarged liver, fatigue, pain in the upper right abdomen, abdominal swelling (ascites), enlarged blood vessels just beneath the skin's surface, enlarged breasts in men, enlarged spleen, red palms, and yellowing of the skin and eyes (jaundice), pruritus, dark urine color, pale stool color nausea or vomiting, loss of appetite, and tendency to bruise easily. Testing for liver diseases can involve blood tests, imaging of the liver, and biopsy of the liver. An individual is at increased risk of a liver disease if the subject has at least one known risk-factor (e.g., genetic factor such as a disease-causing mutation) placing individuals with that risk factor at a statistically significant greater risk of developing the disease than individuals without the risk factor. Risk factors for liver diseases are also well known and can include, for example, excessive alcohol use, obesity, high cholesterol, high levels of triglycerides in the blood, polycystic ovary syndrome, sleep apnea, type 2 diabetes, underactive thyroid (hypothyroidism), underactive pituitary gland (hypopituitarism), and metabolic syndromes including raised blood lipids.

The present disclosure provides methods of treating a subject having liver disease, the methods comprising administering an RNF213 inhibitor to the subject.

The present disclosure also provides methods of treating a subject having fatty liver disease (such as AFLD or NAFLD), the methods comprising administering an RNF213 inhibitor to the subject.

The present disclosure also provides methods of treating a subject having hepatocellular carcinoma, the methods comprising administering an RNF213 inhibitor to the subject.

The present disclosure also provides methods of treating a subject having liver cirrhosis, the methods comprising administering an RNF213 inhibitor to the subject.

The present disclosure also provides methods of treating a subject having liver fibrosis, the methods comprising administering an RNF213 inhibitor to the subject.

The present disclosure also provides methods of treating a subject having simple steatosis, steatohepatitis, or NASH, the methods comprising administering an RNF213 inhibitor to the subject.

In some embodiments, the RNF213 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an RNF213 nucleic acid molecule, such as an mRNA molecule. In some embodiments, the inhibitory nucleic acid molecules hybridize to a sequence within an RNF213 genomic nucleic acid molecule or mRNA molecule and decreases expression of the RNF213 polypeptide in a cell in the subject. In some embodiments, the RNF213 inhibitor comprises an antisense RNA that hybridizes to an RNF213 genomic nucleic acid molecule or mRNA molecule and decreases expression of the RNF213 polypeptide in a cell in the subject. In some embodiments, the RNF213 inhibitor comprises an siRNA that hybridizes to an RNF213 genomic nucleic acid molecule or mRNA molecule and decreases expression of the RNF213 polypeptide in a cell in the subject. In some embodiments, the RNF213 inhibitor comprises an shRNA that hybridizes to an RNF213 genomic nucleic acid molecule or mRNA molecule and decreases expression of the RNF213 polypeptide in a cell in the subject.

In some embodiments, the antisense nucleic acid molecules comprise or consist of any of the nucleotide sequences represented by SEQ ID NOs: 82-20602. In some embodiments, the siRNA molecules comprise or consist of any of the nucleotide sequences (sense and antisense strands) represented by SEQ ID NOs: 20603-64588 (e.g., the sense strand is, for example, SEQ ID NO:20603 and the corresponding antisense strand is SEQ ID NO:20604; the sense strand is, for example, SEQ ID NO:20605 and the corresponding antisense strand is SEQ ID NO:20606; etc.).

The inhibitory nucleic acid molecules disclosed herein can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules disclosed herein can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N₆-methyladenosine, inosine, and N₇-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula: Sense: mN*mN*/i2FN/mN/i2FN/mN/i2FN/mN/i2FN/mN/i2FN/mN/i2FN/mN/i2FN/mN/i2FN/*mN*/32FN/Antisense:/52FN/*/i2FN/*mN/i2FN/mN/i2FN/mN/i2FN/mN/i2FN/mN/i2FN/mN/i2FN/mN/i2FN/mN/i2FN/mN*N*N wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules disclosed herein. In some embodiments, the vectors comprise any one or more of the inhibitory nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the RNF213 inhibitor comprises a nuclease agent that induces one or more nicks or double-strand breaks at a recognition sequence(s) or a DNA-binding protein that binds to a recognition sequence within an RNF213 genomic nucleic acid molecule. The recognition sequence can be located within a coding region of the RNF213 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the RNF213 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an RNF213 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of RNF213 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an RNF213 genomic nucleic acid molecule or it can be a nickase that creates a single-strand break in an RNF213 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of RNF213 genomic nucleic acid molecules can be generated by contacting a cell with a Cas protein and one or more gRNAs that hybridize to one or more gRNA recognition sequences within a target genomic locus in the RNF213 genomic nucleic acid molecule. For example, a gRNA recognition sequence can be located within a region of SEQ ID NO:1. The gRNA recognition sequence can also include or be proximate to a position corresponding to: position 102,917, position 102,391, or position 103,226 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 102,917, position 102,391, or position 103,226 according to SEQ ID NO:1. The gRNA recognition sequence can include or be proximate to the start codon of an RNF213 genomic nucleic acid molecule or the stop codon of an RNF213 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an RNF213 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an RNF213 genomic nucleic acid molecule. An exemplary gRNA is a gRNA effective to direct a Cas enzyme to bind to or cleave an RNF213 genomic nucleic acid molecule, wherein the gRNA comprises a DNA-targeting segment that hybridizes to a gRNA recognition sequence within the RNF213 genomic nucleic acid molecule that includes or is proximate to a position corresponding to: position 102,917, position 102,391, or position 103,226 according to SEQ ID NO:1. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to: position 102,917, position 102,391, or position 103,226 according to SEQ ID NO:1. Other exemplary gRNAs comprise a DNA-targeting segment that hybridizes to a gRNA recognition sequence present within an RNF213 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human RNF213 reference gene are set forth in Table 1 as SEQ ID NOs:62-81.

TABLE 1
Guide RNA Recognition Sequences Near
RNF213 Variation(s)

| | | SEQ |
|---|---|---|
| | | ID |
| Strand | gRNA Recognition Sequence | NO: |
| + | GTGGACCGATTTGCAGTACAGGG | 62 |
| − | GTGCTTTTTCGGTCCGGCAATGG | 63 |
| + | CACGTGGTACCATTGCCGGACGG | 64 |
| − | GAATCTGTAACGGCAGATGAAGG | 65 |
| − | TTGTTCCCGGAACGGTGAGAAGG | 66 |
| + | GGACCCTTGCTGCTACGAAAAGG | 67 |
| + | ATCCAATTCCCCGCGGAGCATGG | 68 |
| − | GTCGCCAACCTCGGTGGGCGCGG | 69 |
| + | CTCCACAATGGCGTCGGCCTCGG | 70 |
| − | AGGTCACGGTGAAACTCATCTGG | 71 |
| + | AGGGATTTACTACCGGCTTCCGG | 72 |
| + | AGTCGGTAAGAATGAACAAGGGG | 73 |
| − | TCCCGGATGACTCACCATAGAGG | 74 |
| + | CCCTTGCTGCTACGAAAAGGTGG | 75 |
| + | TTTGCGGGGCAGGATTCCCGAGG | 76 |
| + | ACAATGGCGTCGGCCTCGGAGGG | 77 |
| − | ACTCACTTCTTGGACGGTCCAGG | 78 |
| − | CTCCGAGGCCGACGCCATTGTGG | 79 |
| + | CACAATGGCGTCGGCCTCGGAGG | 80 |
| + | AATTCCCCGCGGAGCATGGCTGG | 81 |

The Cas protein and the gRNA form a complex, and the Cas protein cleaves the target RNF213 genomic nucleic acid molecule. The Cas protein can cleave the nucleic acid molecule at a site within or outside of the nucleic acid sequence present in the target RNF213 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the RNF213 genomic nucleic acid molecule to which a DNA-targeting segment of a gRNA will bind.

Such methods can result, for example, in an RNF213 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the RNF213 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment further comprise detecting the presence or absence of an RNF213 predicted loss-of-function or missense variant nucleic acid molecule encoding a human RNF213 polypeptide in a biological sample from the subject. As used throughout the present disclosure, a “RNF213 predicted loss-of-function variant nucleic acid molecule” is any RNF213 nucleic acid molecule (such as, for example, genomic nucleic acid molecule, mRNA molecule, or cDNA molecule) encoding an RNF213 polypeptide having a partial loss-of-function, a complete loss-of-function, a predicted partial loss-of-function, or a predicted complete loss-of-function.

The present disclosure also provides methods of treating a subject with a therapeutic agent that treats or inhibits a liver disease, wherein the subject is suffering from a liver disease. In some embodiments, the methods comprise determining whether the subject has an RNF213 predicted loss-of-function or missense variant nucleic acid molecule encoding a human RNF213 polypeptide by obtaining or having obtained a biological sample from the subject, and performing or having performed a genotyping assay on the biological sample to determine if the subject has a genotype comprising the RNF213 predicted loss-of-function or missense variant nucleic acid molecule. When the subject is RNF213 reference, the therapeutic agent that treats or inhibits a liver disease is administered or continued to be administered to the subject in a standard dosage amount, and an RNF213 inhibitor is administered to the subject. When the subject is heterozygous for an RNF213 predicted loss-of-function or missense variant, the therapeutic agent that treats or inhibits a liver disease is administered or continued to be administered to the subject in an amount that is the same as or lower than a standard dosage amount, and an RNF213 inhibitor is administered to the subject. The presence of a genotype having the RNF213 predicted loss-of-function or missense variant nucleic acid molecule encoding the human RNF213 polypeptide indicates the subject has a reduced risk of developing a liver disease. In some embodiments, the subject is RNF213 reference. In some embodiments, the subject is heterozygous for an RNF213 predicted loss-of-function or missense variant.

In some embodiments, the methods comprise determining the subject's aggregate burden of having a plurality of RNF213 predicted loss-of-function or missense variant genomic nucleic acid molecules, RNF213 predicted loss-of-function or missense variant mRNA molecules, and/or RNF213 predicted loss-of-function or missense variant cDNA molecules produced from the mRNA molecules, by: performing or having performed a genotyping assay on a biological sample obtained from the subject to determine the subject's aggregate burden. When the subject has a lower aggregate burden, the subject is at a higher risk of developing a liver disease and the subject is administered or continued to be administered the therapeutic agent that treats or inhibits liver disease in a standard dosage amount. When the subject has a greater aggregate burden, the subject is at a lower risk of developing a liver disease and the subject is administered or continued to be administered the therapeutic agent that treats or inhibits liver disease in an amount that is the same as or lower than the standard dosage amount. The greater the aggregate burden, the lower the risk of developing liver disease.

For subjects that are genotyped or determined to be either RNF213 reference or heterozygous for an RNF213 predicted loss-of-function or missense variant, such subjects can be treated with an RNF213 inhibitor, as described herein.

Detecting the presence or absence of an RNF213 predicted loss-of-function or missense variant nucleic acid molecule in a biological sample from a subject and/or determining whether a subject has an RNF213 predicted loss-of-function or missense variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is RNF213 reference, the subject is also administered a therapeutic agent that treats or inhibits liver disease in a standard dosage amount. In some embodiments, when the subject is heterozygous for an RNF213 predicted loss-of-function or missense variant, the subject is also administered a therapeutic agent that treats or inhibits liver disease in a dosage amount that is the same as or lower than a standard dosage amount.

In some embodiments, the treatment methods further comprise detecting the presence or absence of an RNF213 predicted loss-of-function polypeptide in a biological sample from the subject. In some embodiments, when the subject does not have an RNF213 predicted loss-of-function polypeptide, the subject is also administered a therapeutic agent that treats or inhibits liver disease in a standard dosage amount. In some embodiments, when the subject has an RNF213 predicted loss-of-function polypeptide, the subject is also administered a therapeutic agent that treats or inhibits liver disease in a dosage amount that is the same as or lower than a standard dosage amount.

The present disclosure also provides methods of treating a subject with a therapeutic agent that treats or inhibits liver disease, wherein the subject is suffering from liver disease. In some embodiments, the method comprises determining whether the subject has an RNF213 predicted loss-of-function polypeptide by obtaining or having obtained a biological sample from the subject, and performing or having performed an assay on the biological sample to determine if the subject has an RNF213 predicted loss-of-function polypeptide. When the subject does not have an RNF213 predicted loss-of-function polypeptide, the therapeutic agent that treats or inhibits liver disease is administered or continued to be administered to the subject in a standard dosage amount, and a GPAM inhibitor is administered to the subject. When the subject has an RNF213 predicted loss-of-function polypeptide, the therapeutic agent that treats or inhibits liver disease is administered or continued to be administered to the subject in an amount that is the same as or lower than a standard dosage amount, and an RNF213 inhibitor is administered to the subject. The presence of an RNF213 predicted loss-of-function polypeptide indicates the subject has a reduced risk of developing liver disease. In some embodiments, the subject has an RNF213 predicted loss-of-function polypeptide. In some embodiments, the subject does not have an RNF213 predicted loss-of-function polypeptide.

Detecting the presence or absence of an RNF213 predicted loss-of-function polypeptide in a biological sample from a subject and/or determining whether a subject has an RNF213 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit liver disease include, but are not limited to: Disulfiram, Naltrexone, Acamprosate, Prednisone, Prednisone, Azathioprine, Penicillamine, Trientine, Deferoxamine, Ciprofloxacin, Norofloxacin, Ceftriaxone, Ofloxacin, Amoxicillin-clavulanate, Phytonadione, Bumetanide, Furosemide, Hydrochlorothiazide, Chlorothiazide, Amiloride, Triamterene, Spironolactone, Octreotide, Atenolol, Metoprolol, Nadolol, Propranolol, Timolol, and Carvedilol.

Additional examples of liver disease therapeutic agents (e.g., for use in chronic hepatitis C treatment) include, but are not limited to, ribavirin, paritaprevir, simeprevir (Olysio), grazoprevir, ledipasvir, ombitasvir, elbasvir, daclatasvir (Daklinza), dasabuvir, ritonavir, sofosbuvir, velpatasvir, voxilaprevir, glecaprevir, pibrentasvir, peginterferon alfa-2a, peginterferon alfa-2b, and interferon alfa-2b.

Additional examples of liver disease therapeutic agents (e.g., for use in nonalcoholic fatty liver disease) include, but are not limited to, weight loss inducing agents such as orlistat or sibutramine; insulin sensitizing agents such as thiazolidinediones (TZDs), metformin, and meglitinides; lipid lowering agents such as statins, fibrates, and omega-3 fatty acids; 26orrespondin such as, vitamin E, betaine, N-Acetyl-cysteine, lecithin, silymarin, and beta-carotene; anti TNF agents such as pentoxifylline; probiotics, such as VSL #3; and cytoprotective agents such as ursodeoxycholic acid (UDCA). Other suitable treatments include ACE inhibitors/ARBs, oligofructose, and Incretin analogs.

Additional examples of liver disease therapeutic agents (e.g., for use in NASH) include, but are not limited to, OCALIVA® (obeticholic acid), Selonsertib, Elafibranor, Cenicriviroc, GR_MD_02, MGL_3196, IMM124E, arachidyl amido cholanoic acid (ARAMCHOL™), GS0976, Emricasan, Volixibat, NGM282, GS9674, Tropifexor, MN_001, LMB763, BI_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT_3995, Elobixibat, Namodenoson, Foralumab, SAR425899, Sotagliflozin, EDP_305, Isosabutate, Gemcabene, TERN_101, KBP_042, PF_06865571, DUR928, PF_06835919, NGM313, BMS_986171, Namacizumab, CER_209, ND_L02_s0201, RTU_1096, DRX_065, IONIS_DGAT2Rx, INT_767, NC_001, Seladepar, PXL770, TERN_201, NV556, AZD2693, SP_1373, VK0214, Hepastem, TGFTX4, RLBN1127, GKT_137831, RYI_018, CB4209-CB4211, and JH_0920.

In some embodiments, the dose of the therapeutic agents that treat or inhibit a liver disease can be reduced by about 10%, by about 20%, by about 30%, by about 40%, by about 50%, by about 60%, by about 70%, by about 80%, or by about 90% for subjects or subjects that are heterozygous for an RNF213 predicted loss-of-function or missense variant (i.e., a lower than the standard dosage amount) compared to subjects or subjects that are RNF213 reference (who may receive a standard dosage amount). In some embodiments, the dose of the therapeutic agents that treat or inhibit a liver disease can be reduced by about 10%, by about 20%, by about 30%, by about 40%, or by about 50%. In addition, the dose of therapeutic agents that treat or inhibit a liver disease in subjects or subjects that are heterozygous for an RNF213 predicted loss-of-function or missense variant can be administered less frequently compared to subjects or subjects that are RNF213 reference.

Administration of the therapeutic agents that treat or inhibit a liver disease and/or RNF213 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit a liver disease and/or RNF213 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a liver disease, a decrease/reduction in the severity of a liver disease (such as, for example, a reduction or inhibition of development or a liver disease), a decrease/reduction in symptoms and liver disease-related effects, delaying the onset of symptoms and liver disease-related effects, reducing the severity of symptoms of liver disease-related effects, reducing the severity of an acute episode, reducing the number of symptoms and liver disease-related effects, reducing the latency of symptoms and liver disease-related effects, an amelioration of symptoms and liver disease-related effects, reducing secondary symptoms, reducing secondary infections, preventing relapse to a liver disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of a liver disease development/progression (such as, for example, a complete or partial avoidance/inhibition or a delay), and an increased survival time of the affected host animal, following administration of a therapeutic protocol. Treatment of a liver disease encompasses the treatment of subjects already diagnosed as having any form of a liver disease at any clinical stage or manifestation, the delay of the onset or evolution or aggravation or deterioration of the symptoms or signs of a liver disease, and/or preventing and/or reducing the severity of a liver disease.

The present disclosure also provides methods of identifying a subject having an increased risk for developing liver disease. In some embodiments, the method comprises determining or having determined in a biological sample obtained from the subject the presence or absence of an RNF213 predicted loss-of-function or missense variant nucleic acid molecule (such as a genomic nucleic acid molecule, mRNA molecule, and/or cDNA molecule) encoding a human RNF213 polypeptide. When the subject lacks an RNF213 predicted loss-of-function or missense variant nucleic acid molecule (i.e., the subject is genotypically categorized as RNF213 reference), then the subject has an increased risk for developing liver disease. When the subject has an RNF213 predicted loss-of-function or missense variant nucleic acid molecule (i.e., the subject is heterozygous or homozygous for an RNF213 predicted loss-of-function or missense variant), then the subject has a decreased risk for developing liver disease.

Having a single copy of an RNF213 predicted loss-of-function or missense variant nucleic acid molecule is more protective of a subject from developing liver disease than having no copies of an RNF213 predicted loss-of-function or missense variant nucleic acid molecule. Without intending to be limited to any particular theory or mechanism of action, it is believed that a single copy of an RNF213 predicted loss-of-function or missense variant nucleic acid molecule (i.e., heterozygous for an RNF213 predicted loss-of-function or missense variant) is protective of a subject from developing liver disease, and it is also believed that having two copies of an RNF213 predicted loss-of-function or missense variant nucleic acid molecule (i.e., homozygous for an RNF213 predicted loss-of-function or missense variant) may be more protective of a subject from developing liver disease, relative to a subject with a single copy. Thus, in some embodiments, a single copy of an RNF213 predicted loss-of-function or missense variant nucleic acid molecule may not be completely protective, but instead, may be partially or incompletely protective of a subject from developing liver disease. While not desiring to be bound by any particular theory, there may be additional factors or molecules involved in the development of liver disease that are still present in a subject having a single copy of an RNF213 predicted loss-of-function or missense variant nucleic acid molecule, thus resulting in less than complete protection from the development of liver disease.

Determining whether a subject has an RNF213 predicted loss-of-function or missense variant nucleic acid molecule in a biological sample from a subject and/or determining whether a subject has an RNF213 predicted loss-of-function or missense variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of identifying a subject having an increased risk of developing a liver disease wherein the methods comprise determining or having determined the subject's aggregate burden of having one or more RNF213 predicted loss-of-function or missense variant genomic nucleic acid molecules, mRNA molecules, or cDNA molecules described herein, and/or one or more RNF213 predicted loss-of-function variant polypeptides described herein. The greater the aggregate burden the subject has, the lower the risk for developing a liver disease. The lower the aggregate burden the subject has, the greater the risk for developing a liver disease.

In some embodiments, the methods can further comprise determining the subject's aggregate burden of having a predicted loss-of-function or missense variant RNF213 genomic nucleic acid molecule, mRNA molecule, or cDNA molecule produced from an mRNA molecule, and/or a predicted loss-of-function variant RNF213 polypeptide associated with a decreased risk of liver disease. The aggregate burden is the sum of all variants in the RNF213 gene, which can be carried out in an association analysis with liver disease. In some embodiments, the subject is homozygous for one or more predicted loss-of-function or missense variant RNF213 nucleic acid molecules associated with a decreased risk of developing liver disease. In some embodiments, the subject is heterozygous for one or more predicted loss-of-function or missense variant RNF213 nucleic acid molecules associated with a decreased risk of developing liver disease. The result of the association analysis suggests that loss-of-function and missense variants of RNF213 are associated with decreased risk of liver disease. In some embodiments, when a subject is identified as having an increased risk of developing a liver disease based on their aggregate burden, the subject is further treated with a therapeutic agent that treats or inhibits liver diseases and/or an RNF213 inhibitor, as described herein.

In some embodiments, the subject's aggregate burden of having any one or more RNF213 predicted loss-of-function or missense variant nucleic acid molecules represents a weighted sum of a plurality of any of the predicted loss-of-function or missense variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the RNF213 gene where the genetic burden is the number of alleles multiplied by the association estimate with liver disease or related outcome for each allele (e.g., a weighted polygenic burden score). This can include any genetic variants, regardless of their genomic annotation, in proximity to the RNF213 gene (up to 10 Mb around the gene) that show a non-zero association with liver-related traits in a genetic association analysis. In some embodiments, when the subject has an aggregate burden above a desired threshold score, the subject has a lower or decreased risk of developing a liver disease. In some embodiments, when the subject has an aggregate burden below a desired threshold score, the subject has a greater or increased risk of developing a liver disease.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprise the highest weighted aggregate burdens, including, but not limited to the top 10%, top 20%, top 30%, top 40%, or top 50% of aggregate burdens from a subject population. In some embodiments, the genetic variants comprise the genetic variants having association with a liver disease in the top 10%, top 20%, top 30%, top 40%, or top 50% of p-value range for the association. In some embodiments, each of the identified genetic variants comprise the genetic variants having association with a liver disease with p-value of no more than about 10⁻², about 10⁻³, about 10⁻⁴, about 10⁻⁵, about 10′, about 10⁻², about 10′, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with a liver disease with p-value of less than 5×10⁻⁸. In some embodiments, the identified genetic variants comprise genetic variants having association with a liver disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing liver disease, the subject is further treated with a therapeutic agent that treats or inhibits liver disease and/or an RNF213 inhibitor, as described herein. For example, when the subject is RNF213 reference, and therefore has an increased risk for developing liver disease, the subject is administered an RNF213 inhibitor. In some embodiments, such a subject is also administered a therapeutic agent that treats or inhibits liver disease. In some embodiments, when the subject is heterozygous for an RNF213 predicted loss-of-function or missense variant, the subject is administered the therapeutic agent that treats or inhibits liver disease in a dosage amount that is the same as or lower than a standard dosage amount, and is also administered an RNF213 inhibitor. In some embodiments, the subject is RNF213 reference. In some embodiments, the subject is heterozygous for an RNF213 predicted loss-of-function or missense variant. Furthermore, when the subject has a lower aggregate burden for having an RNF213 predicted loss-of-function or missense variant nucleic acid molecule, and therefore has an increased risk for liver disease, the subject is administered a therapeutic agent that treats or inhibits liver disease. In some embodiments, when the subject has a lower aggregate burden for having an RNF213 predicted loss-of-function or missense variant nucleic acid molecule, the subject is administered the therapeutic agent that treats or inhibits liver disease in a dosage amount that is the same as or greater than the standard dosage amount administered to a subject who has a greater aggregate burden for having an RNF213 predicted loss-of-function or missense variant nucleic acid molecule.

The present disclosure also provides methods of detecting the presence or absence of an RNF213 predicted loss-of-function or missense variant genomic nucleic acid molecule in a biological sample from a subject, and/or an RNF213 predicted loss-of-function or missense variant mRNA molecule in a biological sample from a subject, and/or an RNF213 predicted loss-of-function or missense variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the RNF213 variant genomic nucleic acid molecule, RNF213 variant mRNA molecule, and RNF213 variant cDNA molecule are only exemplary sequences. Other sequences for the RNF213 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any RNF213 variant nucleic acid molecule, preliminary processing designed to isolate or enrich the biological sample for the genomic DNA can be employed. A variety of techniques may be used for this purpose. When detecting the level of any RNF213 variant mRNA molecule, different techniques can be used enrich the biological sample with mRNA molecules. Various methods to detect the presence or level of an mRNA molecule or the presence of a particular variant genomic DNA locus can be used.

In some embodiments, detecting a human RNF213 predicted loss-of-function or missense variant nucleic acid molecule in a subject comprises assaying or genotyping a biological sample obtained from the subject to determine whether an RNF213 genomic nucleic acid molecule in the biological sample, and/or an RNF213 mRNA molecule in the biological sample, and/or an RNF213 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an RNF213 predicted loss-of-function or missense variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the nucleotide sequence comprises: a guanine at a position corresponding to position 102,917 according to SEQ ID NO:2 (for genomic nucleic acid molecules); a guanine at a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18 (for mRNA molecules); or a guanine at a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37 (for CDNA molecules).

In some embodiments, the nucleotide sequence comprises: a cytosine at a position corresponding to position 102,391 according to SEQ ID NO:3 (for genomic nucleic acid molecules); a cytosine at a position corresponding to: position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23, (for mRNA molecules); a cytosine at a position corresponding to: position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42 (for CDNA molecules).

In some embodiments, the nucleotide sequence comprises a thymine at a position corresponding to position 103,226 according to SEQ ID NO:4 (for genomic nucleic acid molecules).

In some embodiments, the nucleotide sequence comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof.

In some embodiments, the nucleotide sequence comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof.

In some embodiments, the nucleotide sequence comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof.

In some embodiments, the biological sample comprises a cell or cell lysate. Such methods can further comprise, for example, obtaining a biological sample from the subject comprising an RNF213 genomic nucleic acid molecule or mRNA molecule, and if mRNA, optionally reverse transcribing the mRNA into cDNA. Such assays can comprise, for example determining the identity of these positions of the particular RNF213 nucleic acid molecule. In some embodiments, the method is an in vitro method.

In some embodiments, the determining step, detecting step, or genotyping assay comprises sequencing at least a portion of the nucleotide sequence of the RNF213 genomic nucleic acid molecule, the RNF213 mRNA molecule, or the RNF213 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or genotyping assay comprises sequencing at least a portion of: the nucleotide sequence of the RNF213 genomic nucleic acid molecule in the biological sample, wherein the sequenced portion comprises a position corresponding to position 102,917 according to SEQ ID NO:2, or the complement thereof; the nucleotide sequence of the RNF213 mRNA molecule in the biological sample, wherein the sequenced portion comprises a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; or position 84 according to SEQ ID NO:18, or the complement thereof; and/or the nucleotide sequence of the RNF213 cDNA molecule produced from the mRNA in the biological sample, wherein the sequenced portion comprises a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof. When the sequenced portion of the RNF213 nucleic acid molecule in the biological sample comprises: a guanine at a position corresponding to position 102,917 according to SEQ ID NO:2; a guanine at a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18; or a guanine at a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37, then the RNF213 nucleic acid molecule in the biological sample is an RNF213 predicted loss-of-function or missense variant nucleic acid molecule.

In some embodiments, the determining step, detecting step, or genotyping assay comprises sequencing at least a portion of: the nucleotide sequence of the RNF213 genomic nucleic acid molecule in the biological sample, wherein the sequenced portion comprises a position corresponding to position 102,391 according to SEQ ID NO:3, or the complement thereof; the nucleotide sequence of the RNF213 mRNA molecule in the biological sample, wherein the sequenced portion comprises a position corresponding to: position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof; and/or the nucleotide sequence of the RNF213 cDNA molecule produced from the mRNA in the biological sample, wherein the sequenced portion comprises a position corresponding to: position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof. When the sequenced portion of the RNF213 nucleic acid molecule in the biological sample comprises: a cytosine at a position corresponding to position 102,391 according to SEQ ID NO:3; a cytosine at a position corresponding to: position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; or a cytosine at a position corresponding to: position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42, then the RNF213 nucleic acid molecule in the biological sample is an RNF213 predicted loss-of-function or missense variant nucleic acid molecule.

In some embodiments, the determining step, detecting step, or genotyping assay comprises sequencing at least a portion of the nucleotide sequence of the RNF213 genomic nucleic acid molecule in the biological sample, wherein the sequenced portion comprises a position corresponding to position 103,226 according to SEQ ID NO:4, or the complement thereof. When the sequenced portion of the RNF213 nucleic acid molecule in the biological sample comprises a thymine at a position corresponding to position 103,226 according to SEQ ID NO:4, then the RNF213 nucleic acid molecule in the biological sample is an RNF213 predicted loss-of-function or missense variant nucleic acid molecule.

In some embodiments, the determining step, detecting step, or genotyping assay comprises sequencing at least a portion of the nucleotide sequence of the RNF213 genomic nucleic acid molecule in the biological sample, wherein the sequenced portion comprises a position corresponding to: position 102,917 according to SEQ ID NO:2, or the complement thereof; position 102,391 according to SEQ ID NO:3, or the complement thereof; or position 103,226 according to SEQ ID NO:4. When the sequenced portion of the RNF213 nucleic acid molecule in the biological sample comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4, then the RNF213 nucleic acid molecule in the biological sample is an RNF213 predicted loss-of-function or missense variant nucleic acid molecule.

In some embodiments, the determining step, detecting step, or genotyping assay comprises sequencing at least a portion of the nucleotide sequence of the RNF213 mRNA molecule in the biological sample, wherein the sequenced portion comprises a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; position 84 according to SEQ ID NO:18, or the complement thereof; position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof. When the sequenced portion of the RNF213 nucleic acid molecule in the biological sample comprises: a guanine at a position corresponding to position 11,887 according to SEQ ID NO:12; a guanine at a position corresponding to position 12,036 according to SEQ ID NO:13; a guanine at a position corresponding to position 2,685 according to SEQ ID NO:14; a guanine at a position corresponding to position 1,050 according to SEQ ID NO:15; a guanine at a position corresponding to position 438 according to SEQ ID NO:16; a guanine at a position corresponding to position 112 according to SEQ ID NO:17; a guanine at a position corresponding to position 84 according to SEQ ID NO:18; a cytosine at a position corresponding to position 11,655 according to SEQ ID NO:19; a cytosine at a position corresponding to position 11,804 according to SEQ ID NO:20; a cytosine at a position corresponding to position 2,453 according to SEQ ID NO:21; a cytosine at a position corresponding to position 818 according to SEQ ID NO:22; or a cytosine at a position corresponding to position 206 according to SEQ ID NO:23; then the RNF213 nucleic acid molecule in the biological sample is an RNF213 predicted loss-of-function or missense variant nucleic acid molecule.

In some embodiments, the determining step, detecting step, or genotyping assay comprises sequencing at least a portion of the nucleotide sequence of the RNF213 cDNA molecule produced from the mRNA molecule in the biological sample, wherein the sequenced portion comprises a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; position 84 according to SEQ ID NO:37, or the complement thereof; position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof. When the sequenced portion of the RNF213 nucleic acid molecule in the biological sample comprises: a guanine at a position corresponding to position 11,887 according to SEQ ID NO:31; a guanine at a position corresponding to position 12,036 according to SEQ ID NO:32; a guanine at a position corresponding to position 2,685 according to SEQ ID NO:33; a guanine at a position corresponding to position 1,050 according to SEQ ID NO:34; a guanine at a position corresponding to position 438 according to SEQ ID NO:35; a guanine at a position corresponding to position 112 according to SEQ ID NO:36; a guanine at a position corresponding to position 84 according to SEQ ID NO:37]; a cytosine at a position corresponding to position 11,655 according to SEQ ID NO:38; a cytosine at a position corresponding to position 11,804 according to SEQ ID NO:39; a cytosine at a position corresponding to position 2,453 according to SEQ ID NO:40; a cytosine at a position corresponding to position 818 according to SEQ ID NO:41; or a cytosine at a position corresponding to position 206 according to SEQ ID NO:42; then the RNF213 nucleic acid molecule in the biological sample is an RNF213 predicted loss-of-function or missense variant nucleic acid molecule.

In some embodiments, the determining step, detecting step, or genotyping assay comprises: a) contacting the biological sample with a primer hybridizing to a portion of the nucleotide sequence of the RNF213: genomic nucleic acid molecule that is proximate to a position corresponding to position 102,917 according to SEQ ID NO:2; mRNA molecule that is proximate to a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18; and/or cDNA molecule that is proximate to a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37; b) extending the primer at least through the position of the nucleotide sequence of the RNF213: genomic nucleic acid molecule corresponding to position 102,917 according to SEQ ID NO:2; mRNA molecule corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18; and/or cDNA molecule corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37; and c) determining whether the extension product of the primer comprises a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18; or a guanine at a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37.

In some embodiments, the determining step, detecting step, or genotyping assay comprises: a) contacting the biological sample with a primer hybridizing to a portion of the nucleotide sequence of the RNF213: genomic nucleic acid molecule that is proximate to a position corresponding to position 102,391 according to SEQ ID NO:3; mRNA molecule that is proximate to a position corresponding to: position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and/or cDNA molecule that is proximate to a position corresponding to: position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; b) extending the primer at least through the position of the nucleotide sequence of the RNF213: genomic nucleic acid molecule corresponding to position 102,391 according to SEQ ID NO:3; mRNA molecule corresponding to: position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and/or cDNA molecule corresponding to: position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; and c) determining whether the extension product of the primer comprises: a cytosine at a position corresponding to position 102,391 according to SEQ ID NO:3; a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23; or a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, a cytosine at a position corresponding to position or 206 according to SEQ ID NO:42.

In some embodiments, the determining step, detecting step, or genotyping assay comprises: a) contacting the biological sample with a primer hybridizing to a portion of the nucleotide sequence of the RNF213 genomic nucleic acid molecule that is proximate to a position corresponding to position 103,226 according to SEQ ID NO:4; b) extending the primer at least through the position of the nucleotide sequence of the RNF213 genomic nucleic acid molecule corresponding to position 103,226 according to SEQ ID NO:4; and c) determining whether the extension product of the primer comprises a thymine at a position corresponding to position 103,226 according to SEQ ID NO:4.

In some embodiments, the determining step, detecting step, or genotyping assay comprises: a) contacting the biological sample with a primer hybridizing to a portion of the nucleotide sequence of the RNF213 genomic nucleic acid molecule that is proximate to a position corresponding to: position 102,917 according to SEQ ID NO:2, position 102,391 according to SEQ ID NO:3, or position 103,226 according to SEQ ID NO:4; b) extending the primer at least through the position of the nucleotide sequence of the RNF213 genomic nucleic acid molecule corresponding to: position 102,917 according to SEQ ID NO:2, position 102,391 according to SEQ ID NO:3, or position 103,226 according to SEQ ID NO:4; and c) determining whether the extension product of the primer comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4.

In some embodiments, the determining step, detecting step, or genotyping assay comprises: a) contacting the biological sample with a primer hybridizing to a portion of the nucleotide sequence of the RNF213 mRNA molecule that is proximate to a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; b) extending the primer at least through the position of the nucleotide sequence of the RNF213 mRNA molecule corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and c) determining whether the extension product of the primer comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23.

In some embodiments, the determining step, detecting step, or genotyping assay comprises: a) contacting the biological sample with a primer hybridizing to a portion of the nucleotide sequence of the RNF213 cDNA molecule that is proximate to a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; b) extending the primer at least through the position of the nucleotide sequence of the RNF213 CDNA molecule corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; and c) determining whether the extension product of the primer comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

In some embodiments, the assay comprises sequencing the entire nucleic acid molecule. In some embodiments, only an RNF213 genomic nucleic acid molecule is analyzed. In some embodiments, only an RNF213 mRNA is analyzed. In some embodiments, only an RNF213 cDNA obtained from RNF213 mRNA is analyzed.

In some embodiments, the determining step, detecting step, or genotyping assay comprises: a) amplifying at least a portion of the nucleic acid molecule that encodes the human RNF213 polypeptide, wherein the amplified portion comprises: i) a guanine at a position corresponding to position 102,917 according to SEQ ID NO:2, or the complement thereof; ii) a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; and/or iii) a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: i) a guanine at a position corresponding to position 102,917 according to SEQ ID NO:2, or the complement thereof; ii) a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; and/or iii) a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; or a guanine at a position corresponding to position 84 according to SEQ ID NO:37, or the complement thereof; and d) detecting the detectable label.

In some embodiments, the determining step, detecting step, or genotyping assay comprises: a) amplifying at least a portion of the nucleic acid molecule that encodes the human RNF213 polypeptide, wherein the amplified portion comprises: i) a cytosine at a position corresponding to position 102,391 according to SEQ ID NO:3, or the complement thereof; ii) a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and/or iii) a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: i) a cytosine at a position corresponding to position 102,391 according to SEQ ID NO:3, or the complement thereof; ii) a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and/or iii) a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and d) detecting the detectable label.

In some embodiments, the determining step, detecting step, or genotyping assay comprises: a) amplifying at least a portion of the nucleic acid molecule that encodes the human RNF213 polypeptide, wherein the amplified portion comprises a thymine at a position corresponding to position 103,226 according to SEQ ID NO:4, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 103,226 according to SEQ ID NO:4, or the complement thereof; and d) detecting the detectable label.

In some embodiments, the determining step, detecting step, or genotyping assay comprises: a) amplifying at least a portion of the nucleic acid molecule that encodes the human RNF213 polypeptide, wherein the amplified portion comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and d) detecting the detectable label.

In some embodiments, the determining step, detecting step, or genotyping assay comprises: a) amplifying at least a portion of the nucleic acid molecule that encodes the human RNF213 polypeptide, wherein the amplified portion comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and d) detecting the detectable label.

In some embodiments, the determining step, detecting step, or genotyping assay comprises: a) amplifying at least a portion of the nucleic acid molecule that encodes the human RNF213 polypeptide, wherein the amplified portion comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: i) a guanine at a position corresponding to position 102,917 according to SEQ ID NO:2, or the complement thereof; ii) a guanine at a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; position 84 according to SEQ ID NO:18, or the complement thereof; and/or iii) a guanine at a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: i) a cytosine at a position corresponding to position 102,391 according to SEQ ID NO:3, or the complement thereof; ii) a cytosine at a position corresponding to: position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof; and/or iii) a cytosine at a position corresponding to: position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 103,226 according to SEQ ID NO:4, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an RNF213 variant genomic sequence, variant mRNA sequence, or variant cDNA sequence and not the corresponding RNF213 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an RNF213 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an RNF213 nucleic acid molecule (genomic nucleic acid molecule, mRNA molecule, or cDNA molecule), or complement thereof, within a biological sample comprises a nucleotide sequence comprising: i) a guanine at a position corresponding to position 102,917 according to SEQ ID NO:2 (genomic nucleic acid molecule); ii) a guanine at a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, or position 84 according to SEQ ID NO:18 (for mRNA molecules); or iii) a guanine at a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37 (for CDNA molecules), the biological sample can be subjected to an amplification method using a primer pair that includes a first primer derived from the 5′ flanking sequence adjacent to a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37, and a second primer derived from the 3′ flanking sequence adjacent to a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37 to produce an amplicon that is indicative of the presence of the SNP at positions encoding a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37, and at least 1, 2, 3, 4, 5, 6, 7, 8, 9, 10, or more nucleotides on each side of positions comprising a guanine at a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37.

In some embodiments, to determine whether an RNF213 nucleic acid molecule (genomic nucleic acid molecule, mRNA molecule, or cDNA molecule), or complement thereof, within a biological sample comprises a nucleotide sequence comprising: i) a cytosine at a position corresponding to position 102,391 according to SEQ ID NO:3 (genomic nucleic acid molecule); ii) a cytosine at a position corresponding to: position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23 (for mRNA molecules); or iii) a cytosine at a position corresponding to: position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42 (for CDNA molecules), the biological sample can be subjected to an amplification method using a primer pair that includes a first primer derived from the 5′ flanking sequence adjacent to a cytosine at a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42, and a second primer derived from the 3′ flanking sequence adjacent to a cytosine at a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42 to produce an amplicon that is indicative of the presence of the SNP at positions encoding a cytosine at a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42, and at least 1, 2, 3, 4, 5, 6, 7, 8, 9, 10, or more nucleotides on each side of positions comprising a cytosine at a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42.

In some embodiments, to determine whether an RNF213 nucleic acid molecule (genomic nucleic acid molecule, mRNA molecule, or cDNA molecule), or complement thereof, within a biological sample comprises a nucleotide sequence comprising a thymine at a position corresponding to position 103,226 according to SEQ ID NO:4 (genomic nucleic acid molecule), the biological sample can be subjected to an amplification method using a primer pair that includes a first primer derived from the 5′ flanking sequence adjacent to a thymine at a position corresponding to position 103,226 according to SEQ ID NO:4, and a second primer derived from the 3′ flanking sequence adjacent to a thymine at a position corresponding to position 103,226 according to SEQ ID NO:4 to produce an amplicon that is indicative of the presence of the SNP at positions encoding a thymine at a position corresponding to position 103,226 according to SEQ ID NO:4. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 103,226 according to SEQ ID NO:4, and at least 1, 2, 3, 4, 5, 6, 7, 8, 9, 10, or more nucleotides on each side of positions comprising a thymine at a position corresponding to position 103,226 according to SEQ ID NO:4.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human RNF213 predicted loss-of-function polypeptide comprising performing an assay on a biological sample obtained from a subject to determine whether an RNF213 polypeptide in the subject contains one or more variations that causes the polypeptide to have a loss-of-function (partial or complete) or predicted loss-of-function (partial or complete). The RNF213 predicted loss-of-function polypeptide can be any of the RNF213 variant polypeptides described herein. In some embodiments, the methods detect the presence of RNF213 Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu. In some embodiments, the methods detect the presence of RNF213 Glu3915Gly or Val3838Leu.

In some embodiments, the methods comprise performing an assay on a sample obtained from a subject to determine whether an RNF213 polypeptide in the sample comprises a glycine at a position corresponding to: position 3,915 according to SEQ ID NO:50, position 3,964 according to SEQ ID NO:51, position 822 according to SEQ ID NO:52, position 350 according to SEQ ID NO:53, position 146 according to SEQ ID NO:54, position 37 according to SEQ ID NO:55, or position 28 according to SEQ ID NO:56. In some embodiments, the methods comprise performing an assay on a sample obtained from a subject to determine whether an RNF213 polypeptide in the sample comprises a leucine at a position corresponding to: position 3,838 according to SEQ ID NO:57, position 3,887 according to SEQ ID NO:58, position 745 according to SEQ ID NO:59, position 273 according to SEQ ID NO:60, or position 69 according to SEQ ID NO:61.

In some embodiments, the detecting step comprises sequencing at least a portion of the polypeptide that comprises a position corresponding to: position 3,915 according to SEQ ID NO:50 or SEQ ID NO:43, position 3,964 according to SEQ ID NO:51 or SEQ ID NO:44, position 822 according to SEQ ID NO:52 or SEQ ID NO:45, position 350 according to SEQ ID NO:53 or SEQ ID NO:46, position 146 according to SEQ ID NO:54 or SEQ ID NO:47, position 37 according to SEQ ID NO:55 or SEQ ID NO:48, or position 28 according to SEQ ID NO:56 or SEQ ID NO:49. In some embodiments, the detecting step comprises sequencing at least a portion of the polypeptide that comprises a position corresponding to: position 3,838 according to SEQ ID NO:57 or SEQ ID NO:43, position 3,887 according to SEQ ID NO:58 or SEQ ID NO:44, position 745 according to SEQ ID NO:59 or SEQ ID NO:45, position 273 according to SEQ ID NO:60 or SEQ ID NO:46, or position 69 according to SEQ ID NO:61 or SEQ ID NO:47.

In some embodiments, the detecting step comprises an immunoassay for detecting the presence of a polypeptide that comprises a position corresponding to: position 3,915 according to SEQ ID NO:50 or SEQ ID NO:43, position 3,964 according to SEQ ID NO:51 or SEQ ID NO:44, position 822 according to SEQ ID NO:52 or SEQ ID NO:45, position 350 according to SEQ ID NO:53 or SEQ ID NO:46, position 146 according to SEQ ID NO:54 or SEQ ID NO:47, position 37 according to SEQ ID NO:55 or SEQ ID NO:48, or position 28 according to SEQ ID NO:56 or SEQ ID NO:49. In some embodiments, the detecting step comprises an immunoassay for detecting the presence of a polypeptide that comprises a position corresponding to: position 3,838 according to SEQ ID NO:57 or SEQ ID NO:43, position 3,887 according to SEQ ID NO:58 or SEQ ID NO:44, position 745 according to SEQ ID NO:59 or SEQ ID NO:45, position 273 according to SEQ ID NO:60 or SEQ ID NO:46, or position 69 according to SEQ ID NO:61 or SEQ ID NO:47.

In some embodiments, when the subject does not have an RNF213 predicted loss-of-function polypeptide, the subject has an increased risk for developing a liver disease. In some embodiments, when the subject has an RNF213 predicted loss-of-function polypeptide, the subject has a decreased risk for developing a liver disease.

The present disclosure also provides isolated nucleic acid molecules that hybridize to RNF213 variant genomic nucleic acid molecules, RNF213 variant mRNA molecules, and/or RNF213 variant cDNA molecules (such as any of the genomic variant nucleic acid molecules, mRNA variant molecules, and cDNA variant molecules disclosed herein). In some embodiments, the isolated nucleic acid molecules hybridize to a portion of the RNF213 nucleic acid molecule that includes a position corresponding to: position 102,917 according to SEQ ID NO:2, position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, or position 84 according to SEQ ID NO:37.

In some embodiments, the isolated nucleic acid molecules hybridize to a portion of the RNF213 nucleic acid molecule that includes a position corresponding to: position 102,391 according to SEQ ID NO:3, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42.

In some embodiments, the isolated nucleic acid molecules hybridize to a portion of the RNF213 nucleic acid molecule that includes a position corresponding to position 103,226 according to SEQ ID NO:4.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to RNF213 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to RNF213 variant genomic nucleic acid molecules, RNF213 variant mRNA molecules, and/or RNF213 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human RNF213 polypeptide, wherein the portion comprises a position corresponding to: position 102,917 according to SEQ ID NO:2, or the complement thereof; position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; position 84 according to SEQ ID NO:18, or the complement thereof; position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof. In some embodiments, the alteration-specific probe or alteration-specific primer comprises a nucleotide sequence which is complementary to a portion of a nucleotide sequence comprising positions corresponding to: positions 102,916-102,918 according to SEQ ID NO:2, or the complement thereof; positions 11,886-11,888 according to SEQ ID NO:12, or the complement thereof; positions 12,035-12,037 according to SEQ ID NO:13, or the complement thereof; positions 2,684-2,686 according to SEQ ID NO:14, or the complement thereof; positions 1,049-1,051 according to SEQ ID NO:15, or the complement thereof; positions 437-439 according to SEQ ID NO:16, or the complement thereof; positions 111-113 according to SEQ ID NO:17, or the complement thereof; positions 83-85 according to SEQ ID NO:18, or the complement thereof; positions 11,886-11,888 according to SEQ ID NO:31, or the complement thereof; positions 12,035-12,037 according to SEQ ID NO:32, or the complement thereof; positions 2,684-2,686 according to SEQ ID NO:33, or the complement thereof; positions 1,049-1,051 according to SEQ ID NO:34, or the complement thereof; positions 437-439 according to SEQ ID NO:35, or the complement thereof; or positions 111-113 according to SEQ ID NO:36, or the complement thereof; or positions 83-85 according to SEQ ID NO:37, the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human RNF213 polypeptide, wherein the portion comprises a position corresponding to: position 102,391 according to SEQ ID NO:3, or the complement thereof; position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; position 206 according to SEQ ID NO:23, or the complement thereof; position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof. In some embodiments, the alteration-specific probe or alteration-specific primer comprises a nucleotide sequence which is complementary to a portion of a nucleotide sequence comprising positions corresponding to: positions 102,391-102,393 according to SEQ ID NO:3, or the complement thereof; positions 11,655-11,657 according to SEQ ID NO:19, or the complement thereof; positions 11,804-11,806 according to SEQ ID NO:20, or the complement thereof; positions 2,453-2,455 according to SEQ ID NO:21, or the complement thereof; positions 818-820 according to SEQ ID NO:22, or the complement thereof; positions 206-208 according to SEQ ID NO:23, or the complement thereof; positions 11,655-11,657 according to SEQ ID NO:38, or the complement thereof; positions 11,804-11,806 according to SEQ ID NO:39, or the complement thereof; positions 2,453-2,455 according to SEQ ID NO:40, or the complement thereof; positions 818-820 according to SEQ ID NO:41, or the complement thereof; or positions 206-208 according to SEQ ID NO:42, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human RNF213 polypeptide, wherein the portion comprises a position corresponding to position 103,226 according to SEQ ID NO:4, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the RNF213 variant genomic nucleic acid molecules, RNF213 variant mRNA molecules, and/or RNF213 variant cDNA molecules disclosed herein. The primers described herein can be used to amplify RNF213 variant genomic nucleic acid molecules, RNF213 variant mRNA molecules, or RNF213 variant cDNA molecules, or a fragment thereof.

The present disclosure also provides pairs of primers comprising any of the primers described above. For example, if one of the primers' 3′-ends hybridizes to an adenine at a position corresponding to position 102,917 according to SEQ ID NO:1 (rather than guanine) in a particular RNF213 nucleic acid molecule, then the presence of the amplified fragment would indicate the presence of an RNF213 reference genomic nucleic acid molecule. Conversely, if one of the primers' 3′-ends hybridizes to a guanine at a position corresponding to position 102,917 according to SEQ ID NO:2 (rather than adenine) in a particular RNF213 nucleic acid molecule, then the presence of the amplified fragment would indicate the presence of the RNF213 variant genomic nucleic acid molecule. In some embodiments, the nucleotide of the primer complementary to the guanine at a position corresponding to position 102,917 according to SEQ ID NO:2 can be at the 3′ end of the primer.

If one of the primers' 3′-ends hybridizes to an adenine at a position corresponding to position 11,887 according to SEQ ID NO:5 (rather than guanine at position 11,887) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 11,887 according to SEQ ID NO:12 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 11,887 according to SEQ ID NO:12 can be at the 3′ end of the primer.

If one of the primers' 3′-ends hybridizes to an adenine at a position corresponding to position 12,036 according to SEQ ID NO:6 (rather than guanine at position 12,036) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 12,036 according to SEQ ID NO:13 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 12,036 according to SEQ ID NO:13 can be at the 3′ end of the primer.

If one of the primers' 3′-ends hybridizes to an adenine at a position corresponding to position 2,685 according to SEQ ID NO:7 (rather than guanine at position 2,685) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 2,685 according to SEQ ID NO:14 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 2,685 according to SEQ ID NO:14 can be at the 3′ end of the primer.

If one of the primers' 3′-ends hybridizes to an adenine at a position corresponding to position 1,050 according to SEQ ID NO:8 (rather than guanine at position 1,050) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 1,050 according to SEQ ID NO:15 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 1,050 according to SEQ ID NO:15 can be at the 3′ end of the primer.

If one of the primers' 3′-ends hybridizes to an adenine at a position corresponding to position 438 according to SEQ ID NO:9 (rather than guanine at position 438) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 438 according to SEQ ID NO:16 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 438 according to SEQ ID NO:16 can be at the 3′ end of the primer.

If one of the primers' 3′-ends hybridizes to an adenine at a position corresponding to position 112 according to SEQ ID NO:10 (rather than guanine at position 112) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 112 according to SEQ ID NO:17 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 112 according to SEQ ID NO:17 can be at the 3′ end of the primer.

If one of the primers' 3′-ends hybridizes to an adenine at a position corresponding to position 84 according to SEQ ID NO:11 (rather than guanine at position 84) in a particular RNF213 nucleic acid molecule, then the presence of the amplified fragment would indicate the presence of an RNF213 reference mRNA molecule. Conversely, if one of the primers' 3′-ends hybridizes to a guanine at a position corresponding to position 84 according to SEQ ID NO:18 (rather than adenine) in a particular RNF213 mRNA molecule, then the presence of the amplified fragment would indicate the presence of the RNF213 variant mRNA molecule. In some embodiments, the nucleotide of the primer complementary to the guanine at a position corresponding to position 84 according to SEQ ID NO:18 can be at the 3′ end of the primer.

If one of the primers' 3′-ends hybridizes to an adenine at a position corresponding to position 11,887 according to SEQ ID NO:24 (rather than guanine at position 11,887) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 11,887 according to SEQ ID NO:31 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 11,887 according to SEQ ID NO:31 can be at the 3′ end of the primer.

If one of the primers' 3′-ends hybridizes to an adenine at a position corresponding to position 12,036 according to SEQ ID NO:25 (rather than guanine at position 12,036) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 12,036 according to SEQ ID NO:32 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 12,036 according to SEQ ID NO:32 can be at the 3′ end of the primer.

If one of the primers' 3′-ends hybridizes to an adenine at a position corresponding to position 2,685 according to SEQ ID NO:26 (rather than guanine at position 2,685) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 2,685 according to SEQ ID NO:33 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 2,685 according to SEQ ID NO:33 can be at the 3′ end of the primer.

If one of the primers' 3′-ends hybridizes to an adenine at a position corresponding to position 1,050 according to SEQ ID NO:27 (rather than guanine at position 1,050) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 1,050 according to SEQ ID NO:34 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 1,050 according to SEQ ID NO:34 can be at the 3′ end of the primer.

If one of the primers' 3′-ends hybridizes to an adenine at a position corresponding to position 438 according to CDNA SEQ ID NO:28 (rather than guanine at position 438) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 438 according to SEQ ID NO:35 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 438 according to SEQ ID NO:35 can be at the 3′ end of the primer.

If one of the primers' 3′-ends hybridizes to an adenine at a position corresponding to position 112 according to SEQ ID NO:29 (rather than guanine at position 112) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 112 according to SEQ ID NO:36 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 112 according to SEQ ID NO:36 can be at the 3′ end of the primer.

If one of the primers' 3′-ends hybridizes to an adenine at a position corresponding to position 84 according to SEQ ID NO:30 (rather than guanine at position 84) in a particular RNF213 nucleic acid molecule, then the presence of the amplified fragment would indicate the presence of an RNF213 reference CDNA molecule. Conversely, if one of the primers' 3′-ends hybridizes to a guanine at a position corresponding to position 84 according to SEQ ID NO:37 (rather than adenine) in a particular RNF213 CDNA molecule, then the presence of the amplified fragment would indicate the presence of the RNF213 variant CDNA molecule. In some embodiments, the nucleotide of the primer complementary to the guanine at a position corresponding to position 84 according to SEQ ID NO:37 can be at the 3′ end of the primer.

The present disclosure also provides pairs of primers comprising any of the primers described above. For example, if one of the primers' 3′-ends hybridizes to a guanine at a position corresponding to position 102,391 according to SEQ ID NO:1 (rather than cytosine) in a particular RNF213 nucleic acid molecule, then the presence of the amplified fragment would indicate the presence of an RNF213 reference genomic nucleic acid molecule. Conversely, if one of the primers' 3′-ends hybridizes to a cytosine at a position corresponding to position 102,391 according to SEQ ID NO:3 (rather than guanine) in a particular RNF213 nucleic acid molecule, then the presence of the amplified fragment would indicate the presence of the RNF213 variant genomic nucleic acid molecule. In some embodiments, the nucleotide of the primer complementary to the cytosine at a position corresponding to position 102,391 according to SEQ ID NO:3 can be at the 3′ end of the primer.

If one of the primers' 3′-ends hybridizes to a guanine at a position corresponding to position 11,655 according to SEQ ID NO:5 (rather than cytosine at position 11,655) in a particular RNF213 nucleic acid molecule, then the presence of the amplified fragment would indicate the presence of an RNF213 reference mRNA molecule. Conversely, if one of the primers' 3′-ends hybridizes to a cytosine at a position corresponding to position 11,655 according to SEQ ID NO:19 (rather than guanine) in a particular RNF213 mRNA molecule, then the presence of the amplified fragment would indicate the presence of the RNF213 variant mRNA molecule. In some embodiments, the nucleotide of the primer complementary to the cytosine at a position corresponding to position 11,655 according to SEQ ID NO:19 can be at the 3′ end of the primer.

If one of the primers' 3′-ends hybridizes to a guanine at a position corresponding to position 11,804 according to SEQ ID NO:6 (rather than cytosine at position 11,804) in a particular RNF213 nucleic acid molecule, then the presence of the amplified fragment would indicate the presence of an RNF213 reference mRNA molecule. Conversely, if one of the primers' 3′-ends hybridizes to a cytosine at a position corresponding to position 11,804 according to SEQ ID NO:20 (rather than guanine) in a particular RNF213 mRNA molecule, then the presence of the amplified fragment would indicate the presence of the RNF213 variant mRNA molecule. In some embodiments, the nucleotide of the primer complementary to the cytosine at a position corresponding to position 11,804 according to SEQ ID NO:20 can be at the 3′ end of the primer.

If one of the primers' 3′-ends hybridizes to a guanine at a position corresponding to position 2,453 according to SEQ ID NO:7 (rather than cytosine at position 2,453) in a particular RNF213 nucleic acid molecule, then the presence of the amplified fragment would indicate the presence of an RNF213 reference mRNA molecule. Conversely, if one of the primers' 3′-ends hybridizes to a cytosine at a position corresponding to position 2,453 according to SEQ ID NO:21 (rather than guanine) in a particular RNF213 mRNA molecule, then the presence of the amplified fragment would indicate the presence of the RNF213 variant mRNA molecule. In some embodiments, the nucleotide of the primer complementary to the cytosine at a position corresponding to position 2,453 according to SEQ ID NO:21 can be at the 3′ end of the primer.

If one of the primers' 3′-ends hybridizes to a guanine at a position corresponding to position 818 according to SEQ ID NO:8 (rather than cytosine at position 818) in a particular RNF213 nucleic acid molecule, then the presence of the amplified fragment would indicate the presence of an RNF213 reference mRNA molecule. Conversely, if one of the primers' 3′-ends hybridizes to a cytosine at a position corresponding to position 818 according to SEQ ID NO:22 (rather than guanine) in a particular RNF213 mRNA molecule, then the presence of the amplified fragment would indicate the presence of the RNF213 variant mRNA molecule. In some embodiments, the nucleotide of the primer complementary to the cytosine at a position corresponding to position 818 according to SEQ ID NO:22 can be at the 3′ end of the primer.

If one of the primers' 3′-ends hybridizes to a guanine at a position corresponding to position 206 according to SEQ ID NO:9 (rather than cytosine at position 206) in a particular RNF213 nucleic acid molecule, then the presence of the amplified fragment would indicate the presence of an RNF213 reference mRNA molecule. Conversely, if one of the primers' 3′-ends hybridizes to a cytosine at a position corresponding to position 206 according to SEQ ID NO:23 (rather than guanine) in a particular RNF213 mRNA molecule, then the presence of the amplified fragment would indicate the presence of the RNF213 variant mRNA molecule. In some embodiments, the nucleotide of the primer complementary to the cytosine at a position corresponding to position 206 according to SEQ ID NO:23 can be at the 3′ end of the primer.

If one of the primers' 3′-ends hybridizes to an guanine at a position corresponding to position 11,655 according to SEQ ID NO:24 (rather than cytosine at position 11,655) in a particular RNF213 nucleic acid molecule, then the presence of the amplified fragment would indicate the presence of an RNF213 reference CDNA molecule. Conversely, if one of the primers' 3′-ends hybridizes to a cytosine at a position corresponding to position 11,655 according to SEQ ID NO:38 (rather than guanine) in a particular RNF213 CDNA molecule, then the presence of the amplified fragment would indicate the presence of the RNF213 variant CDNA molecule. In some embodiments, the nucleotide of the primer complementary to the cytosine at a position corresponding to position 11,655 according to SEQ ID NO:38 can be at the 3′ end of the primer.

If one of the primers' 3′-ends hybridizes to an guanine at a position corresponding to position 11,804 according to SEQ ID NO:25 (rather than cytosine at position 11,804) in a particular RNF213 nucleic acid molecule, then the presence of the amplified fragment would indicate the presence of an RNF213 reference CDNA molecule. Conversely, if one of the primers' 3′-ends hybridizes to a cytosine at a position corresponding to position 11,804 according to SEQ ID NO:39 (rather than guanine) in a particular RNF213 CDNA molecule, then the presence of the amplified fragment would indicate the presence of the RNF213 variant CDNA molecule. In some embodiments, the nucleotide of the primer complementary to the cytosine at a position corresponding to position 11,804 according to SEQ ID NO:39 can be at the 3′ end of the primer.

If one of the primers' 3′-ends hybridizes to an guanine at a position corresponding to position 2,453 according to SEQ ID NO:26 (rather than cytosine at position 2,453) in a particular RNF213 nucleic acid molecule, then the presence of the amplified fragment would indicate the presence of an RNF213 reference CDNA molecule. Conversely, if one of the primers' 3′-ends hybridizes to a cytosine at a position corresponding to position 2,453 according to SEQ ID NO:40 (rather than guanine) in a particular RNF213 CDNA molecule, then the presence of the amplified fragment would indicate the presence of the RNF213 variant CDNA molecule. In some embodiments, the nucleotide of the primer complementary to the cytosine at a position corresponding to position 2,453 according to SEQ ID NO:40 can be at the 3′ end of the primer.

If one of the primers' 3′-ends hybridizes to an guanine at a position corresponding to position 818 according to SEQ ID NO:27 (rather than cytosine at position 818) in a particular RNF213 nucleic acid molecule, then the presence of the amplified fragment would indicate the presence of an RNF213 reference CDNA molecule. Conversely, if one of the primers' 3′-ends hybridizes to a cytosine at a position corresponding to position 818 according to SEQ ID NO:41 (rather than guanine) in a particular RNF213 CDNA molecule, then the presence of the amplified fragment would indicate the presence of the RNF213 variant CDNA molecule. In some embodiments, the nucleotide of the primer complementary to the cytosine at a position corresponding to position 818 according to SEQ ID NO:41 can be at the 3′ end of the primer.

If one of the primers' 3′-ends hybridizes to an guanine at a position corresponding to position 206 according to SEQ ID NO:28 (rather than cytosine at position 206) in a particular RNF213 nucleic acid molecule, then the presence of the amplified fragment would indicate the presence of an RNF213 reference CDNA molecule. Conversely, if one of the primers' 3′-ends hybridizes to a cytosine at a position corresponding to position 206 according to SEQ ID NO:42 (rather than guanine) in a particular RNF213 CDNA molecule, then the presence of the amplified fragment would indicate the presence of the RNF213 variant CDNA molecule. In some embodiments, the nucleotide of the primer complementary to the cytosine at a position corresponding to position 206 according to SEQ ID NO:42 can be at the 3′ end of the primer.

The present disclosure also provides pairs of primers comprising any of the primers described above. For example, if one of the primers' 3′-ends hybridizes to an adenine at a position corresponding to position 103,226 according to SEQ ID NO:1 (rather than thymine) in a particular RNF213 nucleic acid molecule, then the presence of the amplified fragment would indicate the presence of an RNF213 reference genomic nucleic acid molecule. Conversely, if one of the primers' 3′-ends hybridizes to a thymine at a position corresponding to position 103,226 according to SEQ ID NO:4 (rather than adenine) in a particular RNF213 nucleic acid molecule, then the presence of the amplified fragment would indicate the presence of the RNF213 variant genomic nucleic acid molecule. In some embodiments, the nucleotide of the primer complementary to the thymine at a position corresponding to position 103,226 according to SEQ ID NO:4 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an RNF213 reference genomic nucleic acid molecule, an RNF213 reference mRNA molecule, and/or an RNF213 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an RNF213 reference genomic nucleic acid molecule is set forth in SEQ ID NO:1. Referring to SEQ ID NO:1, position 102,917 is an adenine. Referring to SEQ ID NO:1, position 102,391 is a guanine. Referring to SEQ ID NO:1, position 103,226 is a cytosine.

A variant genomic nucleic acid molecule of RNF213 exists, wherein the adenine, at position 102,917 is replaced with guanine. The nucleotide sequence of this RNF213 variant genomic nucleic acid molecule is set forth in SEQ ID NO:2.

Another variant genomic nucleic acid molecule of RNF213 exists, wherein the guanine at position 102,391 is replaced with cytosine. The nucleotide sequence of this RNF213 variant genomic nucleic acid molecule is set forth in SEQ ID NO:3.

Another variant genomic nucleic acid molecule of RNF213 exists, wherein the cytosine at position 103,226 is replaced with thymine. The nucleotide sequence of this RNF213 variant genomic nucleic acid molecule is set forth in SEQ ID NO:4.

The nucleotide sequence of an RNF213 reference mRNA molecule is set forth in SEQ ID NO:5. Referring to SEQ ID NO:5, position 11,887 is an adenine. Referring to SEQ ID NO:5, position 11,655 is a guanine. The nucleotide sequence of another RNF213 reference mRNA molecule is set forth in SEQ ID NO:6. Referring to SEQ ID NO:6, position 12,036 is an adenine. Referring to SEQ ID NO:6, position 11,804 is a guanine. The nucleotide sequence of another RNF213 reference mRNA molecule is set forth in SEQ ID NO:7. Referring to SEQ ID NO:7, position 2,685 is an adenine. Referring to SEQ ID NO:7, position 2,453 is a guanine. The nucleotide sequence of another RNF213 reference mRNA molecule is set forth in SEQ ID NO:8. Referring to SEQ ID NO:8, position 1,050 is an adenine. Referring to SEQ ID NO:8, position 818 is a guanine. The nucleotide sequence of another RNF213 reference mRNA molecule is set forth in SEQ ID NO:9. Referring to SEQ ID NO:9, position 438 is an adenine. Referring to SEQ ID NO:9, position 206 is a guanine. The nucleotide sequence of another RNF213 reference mRNA molecule is set forth in SEQ ID NO:10. Referring to SEQ ID NO:10, position 112 is an adenine. The nucleotide sequence of another RNF213 reference mRNA molecule is set forth in SEQ ID NO:11. Referring to SEQ ID NO:11, position 84 is an adenine.

A variant mRNA molecule of RNF213 exists, wherein the adenine at position 11,887 is replaced with guanine. The nucleotide sequence of this RNF213 variant mRNA molecule is set forth in SEQ ID NO:12.

Another variant mRNA molecule of RNF213 exists, wherein the adenine at position 12,036 is replaced with guanine. The nucleotide sequence of this RNF213 variant mRNA molecule is set forth in SEQ ID NO:13.

Another variant mRNA molecule of RNF213 exists, wherein the adenine at position 2,685 is replaced with guanine. The nucleotide sequence of this RNF213 variant mRNA molecule is set forth in SEQ ID NO:14.

Another variant mRNA molecule of RNF213 exists, wherein the adenine at position 1,050 is replaced with guanine. The nucleotide sequence of this RNF213 variant mRNA molecule is set forth in SEQ ID NO:15.

Another variant mRNA molecule of RNF213 exists, wherein the adenine at position 438 is replaced with guanine. The nucleotide sequence of this RNF213 variant mRNA molecule is set forth in SEQ ID NO:16.

Another variant mRNA molecule of RNF213 exists, wherein the adenine at position 112 is replaced with guanine. The nucleotide sequence of this RNF213 variant mRNA molecule is set forth in SEQ ID NO:17.

Another variant mRNA molecule of RNF213 exists, wherein the adenine at position 84 is replaced with guanine. The nucleotide sequence of this RNF213 variant mRNA molecule is set forth in SEQ ID NO:18.

A variant mRNA molecule of RNF213 exists, wherein the adenine at position 11,655 is replaced with cytosine. The nucleotide sequence of this RNF213 variant mRNA molecule is set forth in SEQ ID NO:19.

Another variant mRNA molecule of RNF213 exists, wherein the adenine at position 11,804 is replaced with cytosine. The nucleotide sequence of this RNF213 variant mRNA molecule is set forth in SEQ ID NO:20.

Another variant mRNA molecule of RNF213 exists, wherein the adenine at position 2,453 is replaced with cytosine. The nucleotide sequence of this RNF213 variant mRNA molecule is set forth in SEQ ID NO:21.

Another variant mRNA molecule of RNF213 exists, wherein the adenine at position 818 is replaced with cytosine. The nucleotide sequence of this RNF213 variant mRNA molecule is set forth in SEQ ID NO:22.

Another variant mRNA molecule of RNF213 exists, wherein the adenine at position 206 is replaced with cytosine. The nucleotide sequence of this RNF213 variant mRNA molecule is set forth in SEQ ID NO:23.

The nucleotide sequence of an RNF213 reference cDNA molecule is set forth in SEQ ID NO:24. Referring to SEQ ID NO:24, position 11,887 is an adenine. Referring to SEQ ID NO:24, position 11,655 is a guanine. The nucleotide sequence of another RNF213 reference cDNA molecule is set forth in SEQ ID NO:25. Referring to SEQ ID NO:25, position 12,036 is an adenine. Referring to SEQ ID NO:25, position 11,804 is a guanine. The nucleotide sequence of another RNF213 reference cDNA molecule is set forth in SEQ ID NO:26. Referring to SEQ ID NO:26, position 2,685 is an adenine. Referring to SEQ ID NO:26, position 2,453 is a guanine. The nucleotide sequence of another RNF213 reference cDNA molecule is set forth in SEQ ID NO:27. Referring to SEQ ID NO:27, position 1,050 is an adenine. Referring to SEQ ID NO:27, position 818 is a guanine. The nucleotide sequence of another RNF213 reference cDNA molecule is set forth in SEQ ID NO:28. Referring to SEQ ID NO:28, position 438 is an adenine. Referring to SEQ ID NO:28, position 206 is a guanine. The nucleotide sequence of another RNF213 reference cDNA molecule is set forth in SEQ ID NO:29. Referring to SEQ ID NO:29, position 112 is an adenine. The nucleotide sequence of another RNF213 reference cDNA molecule is set forth in SEQ ID NO:30. Referring to SEQ ID NO:30, position 84 is an adenine.

A variant cDNA molecule of RNF213 exists, wherein the adenine at position 11,887 is replaced with guanine. The nucleotide sequence of this RNF213 variant cDNA molecule is set forth in SEQ ID NO:31.

Another variant cDNA molecule of RNF213 exists, wherein the adenine at position 12,036 is replaced with guanine. The nucleotide sequence of this RNF213 variant cDNA molecule is set forth in SEQ ID NO:32.

Another variant cDNA molecule of RNF213 exists, wherein the adenine at position 2,685 is replaced with guanine. The nucleotide sequence of this RNF213 variant cDNA molecule is set forth in SEQ ID NO:33.

Another variant cDNA molecule of RNF213 exists, wherein the adenine at position 1,050 is replaced with guanine. The nucleotide sequence of this RNF213 variant cDNA molecule is set forth in SEQ ID NO:34.

Another variant cDNA molecule of RNF213 exists, wherein the adenine at position 438 is replaced with guanine. The nucleotide sequence of this RNF213 variant cDNA molecule is set forth in SEQ ID NO:35.

Another variant cDNA molecule of RNF213 exists, wherein the adenine at position 112 is replaced with guanine. The nucleotide sequence of this RNF213 variant cDNA molecule is set forth in SEQ ID NO:36.

Another variant cDNA molecule of RNF213 exists, wherein the adenine at position 84 is replaced with guanine. The nucleotide sequence of this RNF213 variant cDNA molecule is set forth in SEQ ID NO:37.

A variant cDNA molecule of RNF213 exists, wherein the adenine at position 11,655 is replaced with cytosine. The nucleotide sequence of this RNF213 variant cDNA molecule is set forth in SEQ ID NO:38.

Another variant cDNA molecule of RNF213 exists, wherein the adenine at position 11,804 is replaced with cytosine. The nucleotide sequence of this RNF213 variant cDNA molecule is set forth in SEQ ID NO:39.

Another variant cDNA molecule of RNF213 exists, wherein the adenine at position 2,453 is replaced with cytosine. The nucleotide sequence of this RNF213 variant cDNA molecule is set forth in SEQ ID NO:40.

Another variant cDNA molecule of RNF213 exists, wherein the adenine at position 818 is replaced with cytosine. The nucleotide sequence of this RNF213 variant cDNA molecule is set forth in SEQ ID NO:41.

Another variant cDNA molecule of RNF213 exists, wherein the adenine at position 206 is replaced with cytosine. The nucleotide sequence of this RNF213 variant cDNA molecule is set forth in SEQ ID NO:42.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH 2)ₙOCH 3, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:5, or SEQ ID NO:24). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a human RNF213 polypeptide, wherein the nucleotide sequence comprises a guanine at a position corresponding to position 102,917 according to SEQ ID NO:2 means that if the nucleotide sequence of the RNF213 genomic nucleic acid molecule is aligned to the sequence of SEQ ID NO:2, the RNF213 sequence has a guanine residue at the position that corresponds to position 102,917 of SEQ ID NO:2. The same applies for mRNA molecules comprising a nucleotide sequence encoding a human RNF213 polypeptide, wherein the nucleotide sequence comprises a guanine at a position corresponding to position 11,887 according to SEQ ID NO:12, and cDNA molecules comprising a nucleotide sequence encoding a human RNF213 polypeptide, wherein the nucleotide sequence comprises a guanine at a position corresponding to position 11,887 according to SEQ ID NO:31. In other words, these phrases refer to a nucleic acid molecule encoding an RNF213 polypeptide, wherein the genomic nucleic acid molecule has a nucleotide sequence that comprises a guanine residue that is homologous to the guanine residue at position 102,917 of SEQ ID NO:2 (or wherein the mRNA molecule has a nucleotide sequence that comprises a guanine residue that is homologous to the guanine residue at position 11,887 of SEQ ID NO:12, or wherein the cDNA molecule has a nucleotide sequence that comprises a guanine residue that is homologous to the guanine residue at position 11,887 of SEQ ID NO:31). Herein, such a sequence is also referred to as “RNF213 sequence with the Glu3915Gly alteration” or “RNF213 sequence with the Glu3915Gly variation” referring to genomic nucleic acid molecules (or “RNF213 sequence with the A11887G alteration” or “RNF213 sequence with the A11887G variation” referring to mRNA molecules, and “RNF213 sequence with the A11887G alteration” or “RNF213 sequence with the A11887G variation” referring to cDNA molecules). The same can be carried out for all other molecules disclosed herein.

As described herein, a position within an RNF213 genomic nucleic acid molecule that corresponds to position 102,917 according to SEQ ID NO:2, for example, can be identified by performing a sequence alignment between the nucleotide sequence of a particular RNF213 nucleic acid molecule and the nucleotide sequence of SEQ ID NO:2. A variety of computational algorithms exist that can be used for performing a sequence alignment to identify a nucleotide position that corresponds to, for example, position 102,917 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of RNF213 reference polypeptides are set forth in SEQ ID NO:43 (Isoform 1), SEQ ID NO:44 (Isoform 2), SEQ ID NO:45 (Isoform 3), SEQ ID NO:46 (Isoform 4), SEQ ID NO:47 (Isoform 5), SEQ ID NO:48 (Isoform 6), and SEQ ID NO:49 (Isoform 7). Referring to SEQ ID NO:43 (Isoform 1), the RNF213 reference polypeptide is 5,207 amino acids in length. Referring to SEQ ID NO:43, position 3,915 is glutamic acid. Referring to SEQ ID NO:43, position 3,838 is valine. Referring to SEQ ID NO:44 (Isoform 2), the RNF213 reference polypeptide is 5,256 amino acids in length. Referring to SEQ ID NO:44, position 3,964 is glutamic acid. Referring to SEQ ID NO:44, position 3,887 is valine. Referring to SEQ ID NO:45 (Isoform 3), the RNF213 reference polypeptide is 2,114 amino acids in length. Referring to SEQ ID NO:45, position 822 is glutamic acid. Referring to SEQ ID NO:46 (Isoform 4), the RNF213 reference polypeptide is 1,642 amino acids in length. Referring to SEQ ID NO:46, position 350 is glutamic acid. Referring to SEQ ID NO:46, position 273 is valine. Referring to SEQ ID NO:47 (Isoform 5), the RNF213 reference polypeptide is 5,256 amino acids in length. Referring to SEQ ID NO:47, position 146 is glutamic acid. Referring to SEQ ID NO:44, position 69 is valine. Referring to SEQ ID NO:48 (Isoform 6), the RNF213 reference polypeptide is 37 amino acids in length. Referring to SEQ ID NO:49 (Isoform 7), the RNF213 reference polypeptide is 28 amino acids in length.

A set of RNF213 variant polypeptides exists, wherein the glutamic acid at the positions referred to above for the RNF213 reference polypeptides (referring to SEQ ID NO:43, SEQ ID NO:44, SEQ ID NO:45, SEQ ID NO:46, SEQ ID NO:47, SEQ ID NO:48, and SEQ ID NO:49) is replaced by glycine. Referring to SEQ ID NO:50 (Glu3915Gly; Isoform 1), position 3,915 is glycine. Referring to SEQ ID NO:51 (Glu3964Gly; Isoform 2), position 3,964 is glycine. Referring to SEQ ID NO:52 (Glu822Gly; Isoform 3), position 822 is glycine. Referring to SEQ ID NO:53 (Glu350Gly; Isoform 4), position 350 is glycine. Referring to SEQ ID NO:54 (Glu146Gly; Isoform 5), position 146 is glycine. Referring to SEQ ID NO:55 (Glu37Gly; Isoform 6), position 37 is glycine. Referring to SEQ ID NO:56 (Glu28Gly; Isoform 7), position 28 is glycine.

Another set of RNF213 variant polypeptides exists, wherein the valine at the positions referred to above for the RNF213 reference polypeptides (referring to SEQ ID NO:43, SEQ ID NO:44, SEQ ID NO:45, SEQ ID NO:46, SEQ ID NO:47, SEQ ID NO:48, and SEQ ID NO:49) is replaced by leucine. Referring to SEQ ID NO:57 (Val3838Leu; Isoform 1), position 3,838 is leucine. Referring to SEQ ID NO:58 Val3887Leu; Isoform 2), position 3,887 is leucine. Referring to SEQ ID NO:59 (Val745Leu; Isoform 3), position 745 is leucine. Referring to SEQ ID NO:60 (Val273Leu; Isoform 4), position 273 is leucine. Referring to SEQ ID NO:61 (Val69Leu; Isoform 5), position 69 is leucine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit a liver disease for use in the treatment of a liver disease (or for use in the preparation of a medicament for treating a liver disease) in a subject, wherein the subject has any of the genomic nucleic acid molecules, mRNA molecules, and/or cDNA molecules encoding a human RNF213 polypeptide described herein. The therapeutic agents that treat or inhibit a liver disease can be any of the therapeutic agents that treat or inhibit a liver disease described herein.

In some embodiments, the subject comprises: i) a genomic nucleic acid molecule having a nucleotide sequence encoding a human RNF213 polypeptide, wherein the nucleotide sequence comprises a guanine at a position corresponding to position 102,917 according to SEQ ID NO:2, or the complement thereof; ii) an mRNA molecule having a nucleotide sequence encoding a human RNF213 polypeptide, wherein the nucleotide sequence comprises a guanine at a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; or position 84 according to SEQ ID NO:18, or the complement thereof; or iii) an cDNA molecule having a nucleotide sequence encoding a human RNF213 polypeptide, wherein the nucleotide sequence comprises a guanine at a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof; or iv) an RNF213 polypeptide that comprises a glycine at a position corresponding to: position 3,915 according to SEQ ID NO:50, position 3,964 according to SEQ ID NO:51, position 822 according to SEQ ID NO:52, position 350 according to SEQ ID NO:53, position 146 according to SEQ ID NO:54, position 37 according to SEQ ID NO:55, or position 28 according to SEQ ID NO:56.

In some embodiments, the subject comprises: a genomic nucleic acid molecule having a nucleotide sequence encoding a human RNF213 polypeptide, wherein the nucleotide sequence comprises a guanine at a position corresponding to position 102,917 according to SEQ ID NO:2, or the complement thereof; an mRNA molecule having a nucleotide sequence encoding a human RNF213 polypeptide, wherein the nucleotide sequence comprises a guanine at a position corresponding to position 11,887 according to SEQ ID NO:12, or the complement thereof; a cDNA molecule having a nucleotide sequence encoding a human RNF213 polypeptide, wherein the nucleotide sequence comprises a guanine at a position corresponding to position 11,887 according to SEQ ID NO:31, or the complement thereof; or an RNF213 polypeptide that comprises a glycine at a position corresponding to position 3,915 according to SEQ ID NO:50.

In some embodiments, the subject comprises: i) a genomic nucleic acid molecule having a nucleotide sequence encoding a human RNF213 polypeptide, wherein the nucleotide sequence comprises a cytosine at a position corresponding to position 102,391 according to SEQ ID NO:3, or the complement thereof; ii) an mRNA molecule having a nucleotide sequence encoding a human RNF213 polypeptide, wherein the nucleotide sequence comprises a cytosine at a position corresponding to: position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof; iii) an CDNA molecule having a nucleotide sequence encoding a human RNF213 polypeptide, wherein the nucleotide sequence comprises a cytosine at a position corresponding to: position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof; or iv) an RNF213 polypeptide that comprises a leucine at a position corresponding to: position 3,838 according to SEQ ID NO:57, position 3,887 according to SEQ ID NO:58, position 745 according to SEQ ID NO:59, position 273 according to SEQ ID NO:60, or position 69 according to SEQ ID NO:61.

In some embodiments, the subject comprises: a genomic nucleic acid molecule having a nucleotide sequence encoding a human RNF213 polypeptide, wherein the nucleotide sequence comprises a cytosine at a position corresponding to position 102,391 according to SEQ ID NO:3, or the complement thereof; an mRNA molecule having a nucleotide sequence encoding a human RNF213 polypeptide, wherein the nucleotide sequence comprises a cytosine at a position corresponding to position 11,655 according to SEQ ID NO:19, or the complement thereof; a cDNA molecule having a nucleotide sequence encoding a human RNF213 polypeptide, wherein the nucleotide sequence comprises a cytosine at a position corresponding to position 11,655 according to SEQ ID NO:38, or the complement thereof; or an RNF213 polypeptide that comprises a leucine at a position corresponding to position 3,838 according to SEQ ID NO:57.

In some embodiments, the subject comprises a genomic nucleic acid molecule having a nucleotide sequence encoding a human RNF213 polypeptide, wherein the nucleotide sequence comprises a thymine at a position corresponding to position 103,226 according to SEQ ID NO:4, or the complement thereof.

The present disclosure also provides RNF213 inhibitors for use in the treatment of a liver disease (or for use in the preparation of a medicament for treating a liver disease) in a subject, wherein the subject has any of the genomic nucleic acid molecules, mRNA molecules, and/or cDNA molecules encoding a human RNF213 polypeptide described herein. The RNF213 inhibitors can be any of the RNF213 inhibitors described herein.

In some embodiments, the subject comprises: i) a genomic nucleic acid molecule having a nucleotide sequence encoding a human RNF213 polypeptide, wherein the nucleotide sequence comprises a guanine at a position corresponding to position 102,917 according to SEQ ID NO:2, or the complement thereof; ii) an mRNA molecule having a nucleotide sequence encoding a human RNF213 polypeptide, wherein the nucleotide sequence comprises a guanine at a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; or position 84 according to SEQ ID NO:18, or the complement thereof; or iii) an cDNA molecule having a nucleotide sequence encoding a human RNF213 polypeptide, wherein the nucleotide sequence comprises a guanine at a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof; or iv) an RNF213 polypeptide that comprises a glycine at a position corresponding to: position 3,915 according to SEQ ID NO:50, position 3,964 according to SEQ ID NO:51, position 822 according to SEQ ID NO:52, position 350 according to SEQ ID NO:53, position 146 according to SEQ ID NO:54, position 37 according to SEQ ID NO:55, or position 28 according to SEQ ID NO:56.

In some embodiments, the subject comprises: a genomic nucleic acid molecule having a nucleotide sequence encoding a human RNF213 polypeptide, wherein the nucleotide sequence comprises a guanine at a position corresponding to position 102,917 according to SEQ ID NO:2, or the complement thereof; an mRNA molecule having a nucleotide sequence encoding a human RNF213 polypeptide, wherein the nucleotide sequence comprises a guanine at a position corresponding to position 11,887 according to SEQ ID NO:12, or the complement thereof; a cDNA molecule having a nucleotide sequence encoding a human RNF213 polypeptide, wherein the nucleotide sequence comprises a guanine at a position corresponding to position 11,887 according to SEQ ID NO:31, or the complement thereof; or an RNF213 polypeptide that comprises a glycine at a position corresponding to position 3,915 according to SEQ ID NO:50.

In some embodiments, the subject comprises: i) a genomic nucleic acid molecule having a nucleotide sequence encoding a human RNF213 polypeptide, wherein the nucleotide sequence comprises a cytosine at a position corresponding to position 102,391 according to SEQ ID NO:3, or the complement thereof; ii) an mRNA molecule having a nucleotide sequence encoding a human RNF213 polypeptide, wherein the nucleotide sequence comprises a cytosine at a position corresponding to: position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof; iii) an CDNA molecule having a nucleotide sequence encoding a human RNF213 polypeptide, wherein the nucleotide sequence comprises a cytosine at a position corresponding to: position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof; or iv) an RNF213 polypeptide that comprises a leucine at a position corresponding to: position 3,838 according to SEQ ID NO:57, position 3,887 according to SEQ ID NO:58, position 745 according to SEQ ID NO:59, position 273 according to SEQ ID NO:60, or position 69 according to SEQ ID NO:61.

In some embodiments, the subject comprises: a genomic nucleic acid molecule having a nucleotide sequence encoding a human RNF213 polypeptide, wherein the nucleotide sequence comprises a cytosine at a position corresponding to position 102,391 according to SEQ ID NO:3, or the complement thereof; an mRNA molecule having a nucleotide sequence encoding a human RNF213 polypeptide, wherein the nucleotide sequence comprises a cytosine at a position corresponding to position 11,655 according to SEQ ID NO:19, or the complement thereof; a cDNA molecule having a nucleotide sequence encoding a human RNF213 polypeptide, wherein the nucleotide sequence comprises a cytosine at a position corresponding to position 11,655 according to SEQ ID NO:38, or the complement thereof; or an RNF213 polypeptide that comprises a leucine at a position corresponding to position 3,838 according to SEQ ID NO:57.

In some embodiments, the subject comprises a genomic nucleic acid molecule having a nucleotide sequence encoding a human RNF213 polypeptide, wherein the nucleotide sequence comprises a thymine at a position corresponding to position 103,226 according to SEQ ID NO:4, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Loss of Function of the Gene Encoding RNF213 is Associated with Lower ALT, AST, and Protection Against Liver Disease

To identify genetic factors contributing to chronic liver disease, imputed genotype data, exome sequence data, and electronic health records were analyzed of 597,856 participants of European ancestry in the UK Biobank cohort (UKB), the Geisinger Health System MyCode Community Health Initiative cohort study (GHS) and Mount Sinai's BioMe Personalized Medicine Cohort (SINAI). A discovery analysis in UKB and GHS was performed to identify new genetic variants and genes associated with liver injury as measured by aspartate aminotransferase (AST) and alanine aminotransferase (ALT), which are widely used measures of liver damage. Statistically significant findings were subsequently evaluated for their relationship with a wide range of liver diseases in UKB, GHS, SINAI, The University of Pennsylvania Penn Medicine BioBank (UPENN-PMBB) and the Malmo Diet and Cancer Study (MDCS).

To discover protective genes for liver disease, a 2-step approach was adopted. In the first step, a genome wide analysis study of circulating AST and ALT levels was carried out to identify genes with common protein-coding variant associated at genome wide significance level with AST or ALT. In the second step, the association was determined between the burden of rare loss-of-function alleles in genes from step 1 and ALT or AST; to triangulate the evidence that the identified genes are causal.

In Step 1, the genome wide meta-analysis of AST and ALT using an imputed dataset of 11,914,698 variants in over 500,000 individuals of European ancestry identified 784 genome wide significant regions. One of the genome wide significant loci comprised the gene RNF213 and contained multiple associated coding variants driving the association signals at this locus. The strongest variant for AST was a missense variant in RNF213 rs61740658 (NP_001243000.2, Glu3915Gly). The strongest variant for ALT was an intronic variant rs36103733 (17:80364093:C:T) in perfect linkage disequilibrium with a missense variant in RNF213, rs35332090 (NP_001243000.2, Val3838Leu). After performing fine-mapping with the FINEMAP software, 2 additional coding variants in the 95% credible set of causal variants were identified: rs12944385 (NP_001243000.2, Lys4732Glu) with Posterior probability of 0.20 for ALT and 0.42 for AST, and rs72849841 (NP_066005.2, Pro729Leu) with posterior probability of 0.55 for AST. Association statistics for these variants are listed in Table 2.

TABLE 2
Multiple common coding variants in RNF213 are associated with
liver enzymes. Results are shown in standard deviation units

| | | Per allele beta | | Genotype counts, | |
|---|---|---|---|---|---|
| | | (95% confidence | | RR\|RA\|AA | |
| Genetic exposure | Outcome | interval) in SD | P-value | genotypes | AAF |
| p.Pro729Leu | ALT | −0.01 | 0.00049 | 427,883\|116,000\|8,003 | 0.1196 |
| | | (−0.01, −0.00) | | | |
| p.Pro729Leu | AST | −0.02 | 1.80E−10 | 425,799\|115,453\|7,962 | 0.1196 |
| | | (−0.02, −0.01) | | | |
| p.Glu3915Gly | ALT | −0.02 | 4.40E−10 | 468,399\|79,981\|3,506 | 0.07881 |
| | | (−0.03, −0.01) | | | |
| p.Glu3915Gly | AST | −0.03 | 4.40E−15 | 466,122\|79,608\|3,484 | 0.07882 |
| | | (−0.03, −0.02) | | | |
| 17:80364093:C:T | ALT | −0.02 | 2.20E−10 | 452,198\|94,573\|5,115 | 0.09495 |
| | | (−0.02, −0.01) | | | |
| 17:80364093:C:T | AST | −0.02 | 2.90E−14 | 449,995\|94,131\|5,088 | 0.09496 |
| | | (−0.03, −0.02) | | | |
| p.Val3838Leu | ALT | −0.02 | 6.00E−10 | 451,847\|94,898\|5,141 | 0.09529 |
| | | (−0.02, −0.01) | | | |
| p.Val3838Leu | AST | −0.02 | 2.80E−14 | 449,647\|94,452\|5,115 | 0.0953 |
| | | (−0.03, −0.02) | | | |
| p.Lys4732Glu | ALT | −0.04 | 7.10E−07 | 538,796\|13,002\|87 | 0.01194 |
| | | (−0.05, −0.02) | | | |
| p.Lys4732Glu | AST | −0.05 | 1.60E−08 | 536,195\|12,931\|87 | 0.01193 |
| | | (−0.06, −0.03) | | | |

RR indicates the number of individuals in the population studies carrying no alternative alleles; RA indicates the number of individuals carrying one or more heterozygous alternative alleles; AA indicates the number of individuals carrying one or more homozygous alternative alleles; The alternative allele is the allele causing loss of function or change in amino acid as coded following HGVS recommendations, for 17:80364093:C:T, C is the reference allele, T is the alternative and effect allele for which the summary statistics are reported. SD indicates standard deviation units; AAF indicates the alternative allele frequency.

In step 2, the association for the burden of rare (AAF<1%) predicted loss-of-function (pLOF) variants in RNF213 gene with ALT and AST was estimated using exome sequence data. In this analysis, 1,773 carriers of a pLOF variants in RNF213 had significantly reduced circulating AST and ALT levels as shown in Table 3.

TABLE 3
The burden of loss of function variants for RNF213 is significantly
associated with reduced circulating ALT and AST levels

| | | Per allele beta | | Genotype counts, | |
|---|---|---|---|---|---|
| | | (95% confidence | | RR\|RA\|AA | |
| Genetic exposure | Outcome | interval) | P-value | genotypes | AAF |
| RNF213 pLOF | ALT* | −0.10 | 1.60E−05 | 515,562\|1,772\|1 | 0.17% |
| | | (−0.14, −0.05) | | | |
| RNF213 pLOF | AST* | −0.07 | 0.00088 | 512,981\|1,755\|1 | 0.17% |
| | | (−0.12, −0.03) | | | |

*The associations were additionally corrected for the common fine-mapped signals for ALT or AST respectively to indicate statistical independence from results described in Table 1. RR indicates the number of individuals in the population studies carrying no alternative alleles; RA indicates the number of individuals carrying one or more heterozygous alternative alleles; AA indicates the number of individuals carrying one or more homozygous alternative alleles; The alternative allele is the allele causing loss of function or change in amino acid as coded following HGVS recommendations; SD indicates standard deviation units; AAF indicates the alternative allele frequency; pLOF indicates predicted loss of function.

The association between the burden of loss-of-function variants and AST or ALT was driven by multiple loss of function variants in RNF213 (see, Table 4). These results indicate the common coding alleles described in Table 3 are causing loss of function in RNF213.

TABLE 4
List of predicted loss-of-function variants in RNF213 included in the analysis - Chr:position:ref:alt
indicates the position of the genetic variant on chromosome (chr) with reference (ref) and
it's alternative (alt) allele on build 38 of the Human Genome Reference Consortium

| Variant, chr:position:ref:alt | Transcript IDs | Protein or cDNA change | AAF |
|---|---|---|---|
| 17:80263682:A:G | ENST00000582970, | hgvsp: p.Met1?, | 8.00E−06 |
| | ENST00000319921, | p.Met1?, | |
| | ENST00000508628 | p.Met1? | |
| 17:80287895:TGCCA:T | ENST00000582970, | hgvsp: p.Ser116fs, | 8.00E−06 |
| | ENST00000319921, | p.Ser116fs, | |
| | ENST00000508628 | p.Ser165fs | |
| 17:80288062:GCGACA:G | ENST00000582970, | hgvsp: p.Asp171fs, | 2.10E−05 |
| | ENST00000319921, | p.Asp171fs, | |
| | ENST00000508628 | p.Asp220fs | |
| 17:80288325:C:T | ENST00000582970, | hgvsp: p.Gln258*, | 1.43E−05 |
| | ENST00000319921, | p.Gln258*, | |
| | ENST00000508628 | p.Gln307* | |
| 17:80289797:CAAAA:C | ENST00000582970, | hgvsp: p.Lys359fs, | 8.00E−06 |
| | ENST00000319921, | p.Lys359fs, | |
| | ENST00000508628 | p.Lys408fs | |
| 17:80289797:C:CA | ENST00000582970, | hgvsp: p.Asn360fs, | 6.27E−06 |
| | ENST00000319921, | p.Asn360fs, | |
| | ENST00000508628 | p.Asn409fs | |
| 17:80290616:ATC:A | ENST00000582970, | hgvsp: p.Leu389fs, | 3.26E−06 |
| | ENST00000319921, | p.Leu389fs, | |
| | ENST00000508628 | p.Leu438fs | |
| 17:80290641:CT:C | ENST00000582970, | hgvsp: p.Asp396fs, | 8.00E−06 |
| | ENST00000319921, | p.Asp396fs, | |
| | ENST00000508628 | p.Asp445fs | |
| 17:80294733:C:G | ENST00000582970, | hgvsp: p.Tyr495*, | 1.39E−05 |
| | ENST00000319921, | p.Tyr495*, | |
| | ENST00000508628 | p.Tyr544* | |
| 17:80294917:G:T | ENST00000582970, | hgvsp: p.Glu557*, | 4.51E−05 |
| | ENST00000319921, | p.Glu557*, | |
| | ENST00000508628 | p.Glu606* | |
| 17:80294986:C:T | ENST00000582970, | hgvsp: p.Gln580*, | 1.60E−05 |
| | ENST00000319921, | p.Gln580*, | |
| | ENST00000508628 | p.Gln629* | |
| 17:80298324:G:A | ENST00000582970, | hgvsp: p.Trp672*, | 6.85E−06 |
| | ENST00000319921, | p.Trp672*, | |
| | ENST00000508628 | p.Trp721* | |
| 17:80298349:C:T | ENST00000582970, | hgvsp: p.Gln681*, | 2.78E−05 |
| | ENST00000319921, | p.Gln681*, | |
| | ENST00000508628 | p.Gln730* | |
| 17:80298375:CACCTGGCT:C | ENST00000582970, | hgvsp: p.Thr690fs, | 8.00E−06 |
| | ENST00000319921, | p.Thr690fs, | |
| | ENST00000508628 | p.Thr739fs | |
| 17:80307125:TAGGATGTTC:T | ENST00000582970, | hgvsp: p.Asp810fs, | 0.000165683 |
| | ENST00000319921, | p.Asp810fs, | |
| | ENST00000508628 | p.Asp859fs | |
| 17:80309117:GCCAGCCTTAT:G | ENST00000582970, | hgvsp: p.Pro868fs, | 8.00E−06 |
| | ENST00000319921, | p.Pro868fs, | |
| | ENST00000508628 | p.Pro917fs | |
| 17:80313090:AG:A | ENST00000582970, | hgvsp: p.Arg912fs, | 3.10E−05 |
| | ENST00000319921, | p.Arg912fs, | |
| | ENST00000508628 | p.Arg961fs | |
| 17:80313099:C:T | ENST00000582970, | hgvsp: p.Arg915*, | 8.13E−06 |
| | ENST00000319921, | p.Arg915*, | |
| | ENST00000508628 | p.Arg964* | |
| 17:80313162:A:AT | ENST00000582970, | hgvsp: p.Glu937fs, | 6.41E−06 |
| | ENST00000319921, | p.Glu937fs, | |
| | ENST00000508628 | p.Glu986fs | |
| 17:80317251:G:T | ENST00000582970, | hgvsp: p.Gly959*, | 3.75E−05 |
| | ENST00000319921, | p.Gly959*, | |
| | ENST00000508628 | p.Gly1008* | |
| 17:80319424:G:T | ENST00000319921 | hgvsp: p.Glu1046* | 8.00E−06 |
| 17:80319441:ACCCT:A | ENST00000319921 | hgvsp: p.Ser1053fs | 1.60E−05 |
| 17:80319479:A:G | ENST00000319921 | hgvsp: p.Ter1064Trpext*? | 6.86E−06 |
| 17:80340128:C:T | ENST00000582970, | hgvsp: p.Arg1921*, | 9.75E−06 |
| | ENST00000508628 | p.Arg1970* | |
| 17:80340178:C:A | ENST00000582970, | hgvsp: p.Cys1937*, | 8.00E−06 |
| | ENST00000508628 | p.Cys1986* | |
| 17:80340257:G:GC | ENST00000582970, | hgvsp: p.Gly1965fs, | 8.00E−06 |
| | ENST00000508628 | p.Gly2014fs | |
| 17:80343212:C:T | ENST00000582970, | hgvsp: p.Arg2024*, | 9.26E−06 |
| | ENST00000508628 | p.Arg2073* | |
| 17:80343877:GTTTCT:G | ENST00000582970, | hgvsp: p.Leu2070fs, | 1.77E−05 |
| | ENST00000508628 | p.Leu2119fs | |
| 17:80344720:A:T | ENST00000582970, | hgvsp: p.Lys2129*, | 6.85E−06 |
| | ENST00000508628 | p.Lys2178* | |
| 17:80345275:AC:A | ENST00000582970, | hgvsp: p.Met2315fs, | 8.00E−06 |
| | ENST00000508628 | p.Met2364fs | |
| 17:80345381:A:ACCTGTACCAGGGC | ENST00000582970, | hgvsp: p.Leu2354fs, | 2.71E−06 |
| | ENST00000508628 | p.Leu2403fs | |
| 17:80345812:G:T | ENST00000582970, | hgvsp: p.Glu2493*, | 6.85E−06 |
| | ENST00000508628 | p.Glu2542* | |
| 17:80346285:GT:G | ENST00000582970, | hgvsp: p.Phe2651fs, | 3.17E−06 |
| | ENST00000508628 | p.Phe2700fs | |
| 17:80346386:C:A | ENST00000582970, | hgvsp: p.Ser2684*, | 8.00E−06 |
| | ENST00000508628 | p.Ser2733* | |
| 17:80346627:CCT:C | ENST00000582970, | hgvsp: p.Phe2766fs, | 0 |
| | ENST00000508628 | p.Phe2815fs | |
| 17:80346635:TG:T | ENST00000582970, | hgvsp: p.Val2768fs, | 8.00E−06 |
| | ENST00000508628 | p.Val2817fs | |
| 17:80346804:GCAGTGCGCCCGCTT:G | ENST00000582970, | hgvsp: p.Gln2824fs, | 6.85E−06 |
| | ENST00000508628 | p.Gln2873fs | |
| 17:80346805:C:T | ENST00000582970, | hgvsp: p.Gln2824*, | 8.00E−06 |
| | ENST00000508628 | p.Gln2873* | |
| 17:80346838:CAG:C | ENST00000582970, | hgvsp: p.Gln2835fs, | 1.03E−05 |
| | ENST00000508628 | p.Gln2884fs | |
| 17:80347084:ATC:A | ENST00000582970, | hgvsp: p.Ile2917fs, | 8.00E−06 |
| | ENST00000508628 | p.Ile2966fs | |
| 17:80347089:C:A | ENST00000582970, | hgvsp: p.Cys2918*, | 1.03E−05 |
| | ENST00000508628 | p.Cys2967* | |
| 17:80347570:G:T | ENST00000582970, | hgvsp: p.Glu3079*, | 8.00E−06 |
| | ENST00000508628 | p.Glu3128* | |
| 17:80347638:GC:G | ENST00000582970, | hgvsp: p.Leu3102fs, | 2.64E−06 |
| | ENST00000508628 | p.Leu3151fs | |
| 17:80348255:CAG:C | ENST00000582970, | hgvsp: p.Asp3308fs, | 1.60E−05 |
| | ENST00000508628 | p.Asp3357fs | |
| 17:80349881:G:T | ENST00000582970, | hgvsp: p.Glu3355*, | 8.00E−06 |
| | ENST00000508628 | p.Glu3404* | |
| 17:80351683:A:G | ENST00000582970, | hgvsc: c.10185 − 2A > G, | 0.000367849 |
| | ENST00000508628 | c.10332 − 2A > G | |
| 17:80351716:C:T | ENST00000582970, | hgvsp: p.Arg3406*, | 7.12E−06 |
| | ENST00000508628 | p.Arg3455* | |
| 17:80352946:G:A | ENST00000582970, | hgvsp: p.Trp3437*, | 2.41E−06 |
| | ENST00000508628 | p.Trp3486* | |
| 17:80352981:AC:A | ENST00000582970, | hgvsp: p.Leu3450fs, | 8.00E−06 |
| | ENST00000508628 | p.Leu3499fs | |
| 17:80353018:TCA:T | ENST00000582970, | hgvsp: p.Thr3462fs, | 8.00E−06 |
| | ENST00000508628 | p.Thr3511fs | |
| 17:80353020:AC:A | ENST00000582970, | hgvsp: p.Ile3463fs, | 8.00E−06 |
| | ENST00000508628 | p.Ile3512fs | |
| 17:80353040:GC:G | ENST00000582970, | hgvsp: p.Gly3470fs, | 8.00E−06 |
| | ENST00000508628 | p.Gly3519fs | |
| 17:80353044:G:T | ENST00000582970, | hgvsp: p.Gly3470*, | 8.00E−06 |
| | ENST00000508628 | p.Gly3519* | |
| 17:80353049:CT:C | ENST00000582970, | hgvsp: p.Leu3472fs, | 0 |
| | ENST00000508628 | p.Leu3521fs | |
| 17:80353056:G:T | ENST00000582970, | hgvsp: p.Glu3474*, | 8.00E−06 |
| | ENST00000508628 | p.Glu3523* | |
| 17:80353060:G:A | ENST00000582970, | hgvsc: c.10423 + 1G > A, | 7.88E−06 |
| | ENST00000508628 | c.10570 + 1G > A | |
| 17:80353061:T:C | ENST00000582970, | hgvsc: c.10423 + 2T > C, | 8.00E−06 |
| | ENST00000508628 | c.10570 + 2T > C | |
| 17:80353614:CAG:C | ENST00000582970, | hgvsp: p.Glu3510fs, | 2.89E−05 |
| | ENST00000508628 | p.Glu3559fs | |
| 17:80354148:G:T | ENST00000582970, | hgvsp: p.Glu3570*, | 8.00E−06 |
| | ENST00000508628 | p.Glu3619* | |
| 17:80358480:G:C | ENST00000582970, | hgvsc: c.11054 + 1G > C, | 2.71E−06 |
| | ENST00000508628 | c.11201 + 1G > C | |
| 17:80360082:C:G | ENST00000582970, | hgvsp: p.Tyr3692*, | 1.72E−05 |
| | ENST00000508628 | p.Tyr3741* | |
| 17:80360190:C:A | ENST00000582970, | hgvsp: p.Tyr3728*, | 8.00E−06 |
| | ENST00000508628 | p.Tyr3777* | |
| 17:80360208:T:C | ENST00000582970, | hgvsc: c.11200 + 2T > C, | 8.00E−06 |
| | ENST00000508628 | c.H347 + 2T > C | |
| 17:80363608:G:C | ENST00000582970, | hgvsc: c.11569 − 1G > C, | 1.84E−05 |
| | ENST00000508628 | c.11716 − 1G > C | |
| 17:80363668:G:GC | ENST00000582970, | hgvsp: p.Ser3878fs, | 2.49E−05 |
| | ENST00000508628 | p.Ser3927fs | |
| 17:80363686:G:A | ENST00000582970, | hgvsp: p.Trp3882*, | 3.98E−06 |
| | ENST00000508628 | p.Trp3931* | |
| 17:80364554:G:GT | ENST00000582970, | hgvsc: c.11871 + 1_11871 + 2insT, | 8.00E−06 |
| | ENST00000508628 | c.12018 + 1_12018 + 2insT | |
| 17:80367754:C:T | ENST00000582970, | hgvsp: p.Arg3960*, | 5.44E−06 |
| | ENST00000508628 | p.Arg4009* | |
| 17:80367960:G:C | ENST00000582970, | hgvsc: c.11973 − 1G > C, | 3.22E−06 |
| | ENST00000508628 | c.12120 − 1G > C | |
| 17:80369622:GCT:G | ENST00000582970, | hgvsp: p.Leu4095fs, | 2.77E−05 |
| | ENST00000508628 | p.Leu4144fs | |
| 17:80371973:C:CAACT | ENST00000582970, | hgvsp: p.Cys4177fs, | 8.00E−06 |
| | ENST00000508628 | p.Cys4226fs | |
| 17:80372972:C:CA | ENST00000582970, | hgvsc: c.12752 − 3_12752 − 2insA, | 2.55E−06 |
| | ENST00000508628 | c.12899 − 3_12899 − 2insA | |
| 17:80372973:A:T | ENST00000582970, | hgvsc: c.12752 − 2A > T, | 0.000842726 |
| | ENST00000508628 | c.12899 − 2A > T | |
| 17:80372986:G:T | ENST00000582970, | hgvsp: p.Glu4255*, | 8.00E−06 |
| | ENST00000508628 | p.Glu4304* | |
| 17:80373146:AG:A | ENST00000582970, | hgvsp: p.Asp4309fs, | 8.00E−06 |
| | ENST00000508628 | p.Asp4358fs | |
| 17:80373153:TG:T | ENST00000582970, | hgvsp: p.Val4311fs, | 0 |
| | ENST00000508628 | p.Val4360fs | |
| 17:80373166:G:T | ENST00000582970, | hgvsc: c.12942 + 1G > T, | 8.00E−06 |
| | ENST00000508628 | c.13089 + 1G > T | |
| 17:80375769:A:AC | ENST00000582970, | hgvsp: p.Gln4364fs, | 1.00E−05 |
| | ENST00000508628 | p.Gln4413fs | |
| 17:80375834:C:G | ENST00000582970, | hgvsp: p.Tyr4383*, | 8.00E−06 |
| | ENST00000508628 | p.Tyr4432* | |
| 17:80376895:CA:C | ENST00000582970, | hgvsp: p.Thr4482fs, | 0.000132745 |
| | ENST00000508628 | p.Thr4531fs | |
| 17:80376962:C:G | ENST00000582970, | hgvsp: p.Tyr4503*, | 8.57E−05 |
| | ENST00000508628 | p.Tyr4552* | |
| 17:80376964:G:C | ENST00000582970, | hgvsc: c.13510 + 1G > C, | 1.83E−06 |
| | ENST00000508628 | c.13657 + 1G > C | |
| 17:80376964:G:A | ENST00000582970, | hgvsc: c.13510 + 1G > A, | 8.00E−06 |
| | ENST00000508628 | c.13657 + 1G > A | |
| 17:80379619:G:C | ENST00000582970, | hgvsc: c.13546 − 1G > C, | 2.71E−06 |
| | ENST00000508628 | c.13693 − 1G > C | |
| 17:80380829:A:G | ENST00000582970, | hgvsc: c.13641 − 2A > G, | 8.00E−06 |
| | ENST00000508628 | c.13788 − 2A > G | |
| 17:80380844:A:AT | ENST00000582970, | hgvsp: p.Arg4552fs, | 1.58E−05 |
| | ENST00000508628 | p.Arg4601fs | |
| 17:80380912:G:GC | ENST00000582970, | hgvsp: p.Val4577fs, | 1.83E−06 |
| | ENST00000508628 | p.Val4626fs | |
| 17:80381709:CA:C | ENST00000582970, | hgvsp: p.His4654fs, | 8.00E−06 |
| | ENST00000508628 | p.His4703fs | |
| 17:80381728:G:T | ENST00000582970, | hgvsc: c.13978 + 1G > T, | 8.00E−06 |
| | ENST00000508628 | c.14125 + 1G > T | |
| 17:80381728:G:A | ENST00000582970, | hgvsc: c.13978 + 1G > A, | 3.22E−06 |
| | ENST00000508628 | c.14125 + 1G > A | |
| 17:80383030:G:A | ENST00000582970, | hgvsp: p.Trp4677*, | 4.45E−06 |
| | ENST00000508628 | p.Trp4726* | |
| 17:80383685:TAA:T | ENST00000582970, | hgvsp: p.Lys4694fs, | 8.00E−06 |
| | ENST00000508628 | p.Lys4743fs | |
| 17:80383863:C:T | ENST00000582970, | hgvsp: p.Gln4753*, | 1.60E−05 |
| | ENST00000508628 | p.Gln4802* | |
| 17:80385109:TC:T | ENST00000582970, | hgvsp: p.Gln4799fs, | 5.03E−05 |
| | ENST00000508628 | p.Gln4848fs | |
| 17:80386249:G:T | ENST00000582970, | hgvsc: c.14540 − 1G > T, | 1.76E−06 |
| | ENST00000508628 | c.14687 − 1G > T | |
| 17:80386267:A:T | ENST00000582970, | hgvsp: p.Lys4853*, | 8.00E−06 |
| | ENST00000508628 | p.Lys4902* | |
| 17:80386374:TC:T | ENST00000582970, | hgvsp: p.Arg4889fs, | 8.00E−06 |
| | ENST00000508628 | p.Arg4938fs | |
| 17:80386763:CT:C | ENST00000582970, | hgvsp: p.Leu4932fs, | 8.49E−06 |
| | ENST00000508628 | p.Leu4981fs | |
| 17:80386865:CA:C | ENST00000582970, | hgvsp: p.Gln4966fs, | 8.00E−06 |
| | ENST00000508628 | p.Gln5015fs | |
| 17:80386865:C:T | ENST00000582970, | hgvsp: p.Gln4966*, | 2.71E−06 |
| | ENST00000508628 | p.Gln5015* | |
| 17:80389850:AAC:A | ENST00000582970, | hgvsp: p.Thr5075fs, | 3.59E−06 |
| | ENST00000508628 | p.Thr5124fs | |
| 17:80389868:G:A | ENST00000582970, | hgvsp: p.Trp5079*, | 1.45E−05 |
| | ENST00000508628 | p.Trp5128* | |
| 17:80389918:G:A | ENST00000582970, | hgvsc: c.15285 + 1G > A, | 4.87E−05 |
| | ENST00000508628 | c.15432 + 1G > A | |
| 17:80390010:A:G | ENST00000582970, | hgvsc: c.15286 − 2A > G, | 1.07E−05 |
| | ENST00000508628 | c.15433 − 2A > G | |
| 17:80393428:T:TA | ENST00000582970, | hgvsp: p.Leu5186fs, | 8.00E−06 |
| | ENST00000508628 | p.Leu5235fs | |
| 17:80393484:C:T | ENST00000582970, | hgvsp: p.Arg5204*, | 8.54E−06 |
| | ENST00000508628 | p.Arg5253* | |
| 17:80263721:A:AC | ENST00000582970, | hgvsp: p.Lys16fs, | 7.16E−06 |
| | ENST00000319921, | p.Lys16fs, | |
| | ENST00000508628 | p.Lys16fs | |
| 17:80263756:GC:G | ENST00000582970, | hgvsp: p.Pro26fs, | 6.00E−06 |
| | ENST00000319921, | p.Pro26fs, | |
| | ENST00000508628 | p.Pro26fs | |
| 17:80273317:A:AG | ENST00000582970, | hgvsp: p.Cys62fs, | 6.00E−06 |
| | ENST00000319921, | p.Cys62fs, | |
| | ENST00000508628 | p.Cys62fs | |
| 17:80289838:G:A | ENST00000582970, | hgvsc: c.1112 + 1G > A, | 7.73E−06 |
| | ENST00000319921, | c.1112 + 1G > A, | |
| | ENST00000508628 | c.1259 + 1G > A | |
| 17:80291794:CTGTT:C | ENST00000582970, | hgvsp: p.Leu480fs, | 1.70E−05 |
| | ENST00000319921, | p.Leu480fs, | |
| | ENST00000508628 | p.Leu529fs | |
| 17:80295556:G:A | ENST00000582970, | hgvsc: c.1756 − 1G > A, | 1.20E−05 |
| | ENST00000319921, | c.1756 − 1G > A, | |
| | ENST00000508628 | c.1903 − 1G > A | |
| 17:80298476:A:AG | ENST00000582970, | hgvsp: p.Leu725fs, | 6.00E−06 |
| | ENST00000319921, | p.Leu725fs, | |
| | ENST00000508628 | p.Leu774fs | |
| 17:80307182:C:CT | ENST00000582970, | hgvsp: p.Asp829fs, | 6.00E−06 |
| | ENST00000319921, | p.Asp829fs, | |
| | ENST00000508628 | p.Asp878fs | |
| 17:80313066:C:T | ENST00000582970, | hgvsp: p.Gln904*, | 1.20E−05 |
| | ENST00000319921, | p.Gln904*, | |
| | ENST00000508628 | p.Gln953* | |
| 17:80319205:C:T | ENST00000582970, | hgvsp: p.Gln973*, | 6.00E−06 |
| | ENST00000319921, | p.Gln973*, | |
| | ENST00000508628 | p.Gln1022* | |
| 17:80319441:AC:A | ENST00000319921 | hgvsp: p.Ser1053fs | 1.20E−05 |
| 17:80325033:C:T | ENST00000582970, | hgvsp: p.Gln1010*, | 6.00E−06 |
| | ENST00000508628 | p.Gln1059* | |
| 17:80328460:T:TC | ENST00000582970, | hgvsp: p.Lys1168fs, | 6.00E−06 |
| | ENST00000508628 | p.Lys1217fs | |
| 17:80332023:GCAGTAAGA:G | ENST00000582970, | hgvsp: p.Val1180fs, | 6.00E−06 |
| | ENST00000508628 | p.Val1229fs | |
| 17:80332503:C:T | ENST00000582970, | hgvsp: p.Arg1339*, | 4.81E−06 |
| | ENST00000508628 | p.Arg1388* | |
| 17:80334264:CT:C | ENST00000582970, | hgvsp: p.Gly1436fs, | 6.00E−06 |
| | ENST00000508628 | p.Gly1485fs | |
| 17:80337585:G:T | ENST00000582970, | hgvsc: c.4528 − 1G > T, | 1.17E−05 |
| | ENST00000508628 | c.4675 − 1G > T | |
| 17:80337832:G:GATTT | ENST00000582970, | hgvsp: p.Ser1558fs, | 1.20E−05 |
| | ENST00000508628 | p.Ser1607fs | |
| 17:80337836:TC:T | ENST00000582970, | hgvsp: p.Pro1559fs, | 6.00E−06 |
| | ENST00000508628 | p.Pro1608fs | |
| 17:80337923:G:T | ENST00000582970, | hgvsp: p.Glu1587*, | 6.00E−06 |
| | ENST00000508628 | p.Glu1636* | |
| 17:80339340:AC:A | ENST00000582970, | hgvsp: p.Leu1659fs, | 6.00E−06 |
| | ENST00000508628 | p.Leu1708fs | |
| 17:80339467:C:A | ENST00000582970, | hgvsp: p.Tyr1700*, | 6.00E−06 |
| | ENST00000508628 | p.Tyr1749* | |
| 17:80339519:A:T | ENST00000582970, | hgvsp: p.Lys1718*, | 1.20E−05 |
| | ENST00000508628 | p.Lys1767* | |
| 17:80339852:C:T | ENST00000582970, | hgvsp: p.Gln1829*, | 3.46E−06 |
| | ENST00000508628 | p.Gln1878* | |
| 17:80339981:G:GAGGA | ENST00000582970, | hgvsp: p.Val1874fs, | 3.35E−05 |
| | ENST00000508628 | p.Val1923fs | |
| 17:80339981:G:T | ENST00000582970, | hgvsp: p.Glu1872*, | 6.00E−06 |
| | ENST00000508628 | p.Glu1921* | |
| 17:80340018:TG:T | ENST00000582970, | hgvsp: p.Gly1885fs, | 6.00E−06 |
| | ENST00000508628 | p.Gly1934fs | |
| 17:80340129:GA:G | ENST00000582970, | hgvsp: p.Glu1922fs, | 6.00E−06 |
| | ENST00000508628 | p.Glu1971fs | |
| 17:80340224:C:T | ENST00000582970, | hgvsp: p.Gln1953*, | 6.00E−06 |
| | ENST00000508628 | p.Gln2002* | |
| 17:80340344:C:T | ENST00000582970, | hgvsp: p.Arg1993*, | 5.77E−06 |
| | ENST00000508628 | p.Arg2042* | |
| 17:80343242:C:T | ENST00000582970, | hgvsp: p.Arg2034*, | 9.38E−06 |
| | ENST00000508628 | p.Arg2083* | |
| 17:80344840:CAG:C | ENST00000582970, | hgvsp: p.Gln2169fs, | 1.88E−05 |
| | ENST00000508628 | p.Gln2218fs | |
| 17:80344852:C:T | ENST00000582970, | hgvsp: p.Arg2173*, | 8.07E−06 |
| | ENST00000508628 | p.Arg2222* | |
| 17:80345032:CTCTT:C | ENST00000582970, | hgvsp: p.Leu2233fs, | 6.00E−06 |
| | ENST00000508628 | p.Leu2282fs | |
| 17:80345787:G:GT | ENST00000582970, | hgvsp: p.Asp2487fs, | 1.77E−06 |
| | ENST00000508628 | p.Asp2536fs | |
| 17:80346221:CAG:C | ENST00000582970, | hgvsp: p.Glu2630fs, | 4.31E−06 |
| | ENST00000508628 | p.Glu2679fs | |
| 17:80346292:G:T | ENST00000582970, | hgvsp: p.Glu2653*, | 6.00E−06 |
| | ENST00000508628 | p.Glu2702* | |
| 17:80346722:TC:T | ENST00000582970, | hgvsp: p.Arg2797fs, | 1.77E−06 |
| | ENST00000508628 | p.Arg2846fs | |
| 17:80346926:TG:T | ENST00000582970, | hgvsp: p.Glu2865fs, | 6.00E−06 |
| | ENST00000508628 | p.Glu2914fs | |
| 17:80346949:G:GCCCC | ENST00000582970, | hgvsp: p.Asp2872fs, | 6.00E−06 |
| | ENST00000508628 | p.Asp2921fs | |
| 17:80347058:C:CA | ENST00000582970, | hgvsp: p.Glu2909fs, | 6.00E−06 |
| | ENST00000508628 | p.Glu2958fs | |
| 17:80347196:G:GT | ENST00000582970, | hgvsp: p.Asp2955fs, | 6.00E−06 |
| | ENST00000508628 | p.Asp3004fs | |
| 17:80347248:TAGAA:T | ENST00000582970, | hgvsp: p.Arg2972fs, | 6.00E−06 |
| | ENST00000508628 | p.Arg3021fs | |
| 17:80347295:TC:T | ENST00000582970, | hgvsp: p.Phe2987fs, | 6.00E−06 |
| | ENST00000508628 | p.Phe3036fs | |
| 17:80347906:C:CT | ENST00000582970, | hgvsp: p.Cys3192fs, | 2.62E−06 |
| | ENST00000508628 | p.Cys3241fs | |
| 17:80348200:CACAG:C | ENST00000582970, | hgvsp: p.Gln3291fs, | 6.00E−06 |
| | ENST00000508628 | p.Gln3340fs | |
| 17:80349805:CTG:C | ENST00000582970, | hgvsp: p.Cys3330fs, | 7.73E−06 |
| | ENST00000508628 | p.Cys3379fs | |
| 17:80349825:AG:A | ENST00000582970, | hgvsp: p.Val3337fs, | 6.00E−06 |
| | ENST00000508628 | p.Val3386fs | |
| 17:80349847:AC:A | ENST00000582970, | hgvsp: p.Thr3345fs, | 6.00E−06 |
| | ENST00000508628 | p.Thr3394fs | |
| 17:80349870:A:AG | ENST00000582970, | hgvsp: p.Phe3352fs, | 6.00E−06 |
| | ENST00000508628 | p.Phe3401fs | |
| 17:80350394:TA:T | ENST00000582970, | hgvsp: p.Ile3391fs, | 6.00E−06 |
| | ENST00000508628 | p.Ile3440fs | |
| 17:80351719:GA:G | ENST00000582970, | hgvsp: p.Asn3408fs, | 6.00E−06 |
| | ENST00000508628 | p.Asn3457fs | |
| 17:80351795:TC:T | ENST00000582970, | hgvsp: p.His3433fs, | 0 |
| | ENST00000508628 | p.His3482fs | |
| 17:80353644:CCT:C | ENST00000582970, | hgvsp: p.Ser3520fs, | 6.00E−06 |
| | ENST00000508628 | p.Ser3569fs | |
| 17:80358414:TC:T | ENST00000582970, | hgvsp: p.Leu3664fs, | 6.00E−06 |
| | ENST00000508628 | p.Leu3713fs | |
| 17:80360060:G:C | ENST00000582970, | hgvsc: c.11055 − 1G > C, | 1.80E−05 |
| | ENST00000508628 | c.11202 − 1G > C | |
| 17:80360144:G:A | ENST00000582970, | hgvsp: p.Trp3713*, | 1.77E−06 |
| | ENST00000508628 | p.Trp3762* | |
| 17:80363101:G:GT | ENST00000582970, | hgvsp: p.Leu3787fs, | 6.00E−06 |
| | ENST00000508628 | p.Leu3836fs | |
| 17:80363286:G:A | ENST00000582970, | hgvsp: p.Trp3847*, | 2.38E−05 |
| | ENST00000508628 | p.Trp3896* | |
| 17:80363727:T:TC | ENST00000582970, | hgvsp: p.Cys3897fs, | 6.00E−06 |
| | ENST00000508628 | p.Cys3946fs | |
| 17:80368092:G:GTT | ENST00000582970, | hgvsp: p.Leu4036fs, | 6.00E−06 |
| | ENST00000508628 | p.Leu4085fs | |
| 17:80369645:AG:A | ENST00000582970, | hgvsp: p.Arg4102fs, | 6.00E−06 |
| | ENST00000508628 | p.Arg4151fs | |
| 17:80369868:GT:G | ENST00000582970, | hgvsc: c.12425 + 2delT, | 0 |
| | ENST00000508628 | c.12572 + 2delT | |
| 17:80371986:G:A | ENST00000582970, | hgvsc: c.12537 + 1G > A, | 6.76E−06 |
| | ENST00000508628 | c.12684 + 1G > A | |
| 17:80372723:A:AG | ENST00000582970, | hgvsp: p.Gly4249fs, | 1.56E−05 |
| | ENST00000508628 | p.Gly4298fs | |
| 17:80372977:AT:A | ENST00000582970, | hgvsp: p.Met4252fs, | 0.001243 |
| | ENST00000508628 | p.Met4301fs | |
| 17:80375758:AG:A | ENST00000582970, | hgvsc: c.13075 − 1delG, | 0 |
| | ENST00000508628 | c.13222 − 1delG | |
| 17:80375759:G:T | ENST00000582970, | hgvsc: c.13075 − 1G > T, | 6.00E−06 |
| | ENST00000508628 | c.13222 − 1G > T | |
| 17:80375857:AC:A | ENST00000582970, | hgvsp: p.Thr4393fs, | 1.77E−06 |
| | ENST00000508628 | p.Thr4442fs | |
| 17:80376965:T:C | ENST00000582970, | hgvsc: c.13510 + 2T > C, | 6.00E−06 |
| | ENST00000508628 | c.13657 + 2T > C | |
| 17:80379716:T:C | ENST00000582970, | hgvsc: c.13640 + 2T > C, | 1.77E−06 |
| | ENST00000508628 | c.13787 + 2T > C | |
| 17:80380838:GCAGA:G | ENST00000582970, | hgvsp: p.Asp4551fs, | 6.00E−06 |
| | ENST00000508628 | p.Asp4600fs | |
| 17:80380988:G:T | ENST00000582970, | hgvsc: c.13797 + 1G > T, | 1.77E−06 |
| | ENST00000508628 | c.13944 + 1G > T | |
| 17:80385172:G:A | ENST00000582970, | hgvsc: c.14455 + 1G > A, | 6.00E−06 |
| | ENST00000508628 | c.14602 + 1G > A | |
| 17:80385549:C:T | ENST00000582970, | hgvsp: p.Gln4823*, | 6.00E−06 |
| | ENST00000508628 | p.Gln4872* | |
| 17:80386327:C:T | ENST00000582970, | hgvsp: p.Arg4873*, | 6.45E−06 |
| | ENST00000508628 | p.Arg4922* | |
| 17:80386747:GC:G | ENST00000582970, | hgvsp: p.Arg4927fs, | 6.00E−06 |
| | ENST00000508628 | p.Arg4976fs | |
| 17:80389215:C:CGA | ENST00000582970, | hgvsp: p.Leu5015fs, | 6.00E−06 |
| | ENST00000508628 | p.Leu5064fs | |
| 17:80389217:G:GC | ENST00000582970, | hgvsp: p.Gln5016fs, | 6.00E−06 |
| | ENST00000508628 | p.Gln5065fs | |
| 17:80389286:T:TG | ENST00000582970, | hgvsp: p.Gly5040fs, | 1.17E−05 |
| | ENST00000508628 | p.Gly5089fs | |
| 17:80389832:T:TAA | ENST00000582970, | hgvsp: p.Asn5068fs, | 6.00E−06 |
| | ENST00000508628 | p.Asn5117fs | |
| 17:80393452:TG:T | ENST00000582970, | hgvsp: p.Trp5194fs, | 6.00E−06 |
| | ENST00000508628 | p.Trp5243fs | |
| 17:80263675:AGGACCCAT:A | ENST00000582970, | hgvsp: p.Met1fs, | 1.00E−06 |
| | ENST00000319921, | p.Met1fs, | |
| | ENST00000508628 | p.Met1fs | |
| 17:80263739:C:T | ENST00000582970, | hgvsp: p.Gln20*, | 1.00E−06 |
| | ENST00000319921, | p.Gln20*, | |
| | ENST00000508628 | p.Gln20* | |
| 17:80263761:C:CTGCAG | ENST00000582970, | hgvsp: p.Ala29fs, | 1.00E−06 |
| | ENST00000319921, | p.Ala29fs, | |
| | ENST00000508628 | p.Ala29fs | |
| 17:80263778:GGT:G | ENST00000582970, | hgvsc: c.97 + 1_97 + 2delGT, | 1.00E−06 |
| | ENST00000319921, | c.97 + 1_97 + 2delGT, | |
| | ENST00000508628 | c.97 + 1_97 + 2delGT | |
| 17:80263779:GT:G | ENST00000582970, | hgvsc: c.97 + 2delT, | 3.83E−06 |
| | ENST00000319921, | c.97 + 2delT, | |
| | ENST00000508628 | c.97 + 2delT | |
| 17:80263779:G:C | ENST00000582970, | hgvsc: c.97 + 1G > C, | 1.00E−06 |
| | ENST00000319921, | c.97 + 1G > C, | |
| | ENST00000508628 | c.97 + 1G > C | |
| 17:80273364:C:CT | ENST00000582970, | hgvsp: p.Glu75fs, | 1.00E−06 |
| | ENST00000319921, | p.Glu75fs, | |
| | ENST00000508628 | p.Glu75fs | |
| 17:80273374:CT:C | ENST00000582970, | hgvsp: p.Cys78fs, | 1.00E−06 |
| | ENST00000319921, | p.Cys78fs, | |
| | ENST00000508628 | p.Cys78fs | |
| 17:80278790:T:TGCTGC | ENST00000508628 | hgvsp: p.Val99fs | 1.00E−06 |
| 17:80278815:G:T | ENST00000508628 | hgvsp: p.Glu105* | 1.00E−06 |
| 17:80278853:G:GTT | ENST00000508628 | hgvsp: p.Lys118fs | 1.00E−06 |
| 17:80278878:GC:G | ENST00000508628 | hgvsp: p.Ala127fs | 3.00E−06 |
| 17:80278901:CTG:C | ENST00000508628 | hgvsp: p.Cys134fs | 1.00E−06 |
| 17:80287813:A:G | ENST00000582970, | hgvsc: c.262 − 2A > G, | 1.00E−06 |
| | ENST00000319921, | c.262 − 2A > G, | |
| | ENST00000508628 | c.409 − 2A > G | |
| 17:80287918:T:A | ENST00000582970, | hgvsp: p.Leu122*, | 1.00E−06 |
| | ENST00000319921, | p.Leu122*, | |
| | ENST00000508628 | p.Leu171* | |
| 17:80287924:C:A | ENST00000582970, | hgvsp: p.Ser124*, | 1.00E−06 |
| | ENST00000319921, | p.Ser124*, | |
| | ENST00000508628 | p.Ser173* | |
| 17:80287947:G:GCCCT | ENST00000582970, | hgvsp: p.His135fs, | 3.00E−06 |
| | ENST00000319921, | p.His135fs, | |
| | ENST00000508628 | p.His184fs | |
| 17:80287952:GC:G | ENST00000582970, | hgvsp: p.His135fs, | 1.00E−06 |
| | ENST00000319921, | p.His135fs, | |
| | ENST00000508628 | p.His184fs | |
| 17:80287989:C:T | ENST00000582970, | hgvsp: p.Gln146*, | 3.00E−06 |
| | ENST00000319921, | p.Gln146*, | |
| | ENST00000508628 | p.Gln195* | |
| 17:80288076:C:T | ENST00000582970, | hgvsp: p.Gln175*, | 1.00E−06 |
| | ENST00000319921, | p.Gln175*, | |
| | ENST00000508628 | p.Gln224* | |
| 17:80288079:GC:G | ENST00000582970, | hgvsp: p.Gln177fs, | 1.00E−06 |
| | ENST00000319921, | p.Gln177fs, | |
| | ENST00000508628 | p.Gln226fs | |
| 17:80288082:C:T | ENST00000582970, | hgvsp: p.Gln177*, | 7.58E−06 |
| | ENST00000319921, | p.Gln177*, | |
| | ENST00000508628 | p.Gln226* | |
| 17:80288089:T:A | ENST00000582970, | hgvsp: p.Leu179*, | 4.00E−06 |
| | ENST00000319921, | p.Leu179*, | |
| | ENST00000508628 | p.Leu228* | |
| 17:80288124:CAG:C | ENST00000582970, | hgvsp: p.Ser192fs, | 2.80E−06 |
| | ENST00000319921, | p.Ser192fs, | |
| | ENST00000508628 | p.Ser241fs | |
| 17:80288186:T:TG | ENST00000582970, | hgvsp: p.Arg214fs, | 1.00E−06 |
| | ENST00000319921, | p.Arg214fs, | |
| | ENST00000508628 | p.Arg263fs | |
| 17:80288214:A:AC | ENST00000582970, | hgvsp: p.Gly222fs, | 1.00E−06 |
| | ENST00000319921, | p.Gly222fs, | |
| | ENST00000508628 | p.Gly271fs | |
| 17:80288238:G:T | ENST00000582970, | hgvsp: p.Glu229*, | 1.00E−06 |
| | ENST00000319921, | p.Glu229*, | |
| | ENST00000508628 | p.Glu278* | |
| 17:80288265:GC:G | ENST00000582970, | hgvsp: p.Gln239fs, | 1.00E−06 |
| | ENST00000319921, | p.Gln239fs, | |
| | ENST00000508628 | p.Gln288fs | |
| 17:80288337:C:T | ENST00000582970, | hgvsp: p.Gln262*, | 3.00E−06 |
| | ENST00000319921, | p.Gln262*, | |
| | ENST00000508628 | p.Gln311* | |
| 17:80288340:C:T | ENST00000582970, | hgvsp: p.Gln263*, | 1.00E−06 |
| | ENST00000319921, | p.Gln263*, | |
| | ENST00000508628 | p.Gln312* | |
| 17:80288688:TGAAAG:T | ENST00000582970, | hgvsp: p.Met289fs, | 1.00E−06 |
| | ENST00000319921, | p.Met289fs, | |
| | ENST00000508628 | p.Met338fs | |
| 17:80288714:C:T | ENST00000582970, | hgvsp: p.Gln298*, | 2.00E−06 |
| | ENST00000319921, | p.Gln298*, | |
| | ENST00000508628 | p.Gln347* | |
| 17:80288753:C:T | ENST00000582970, | hgvsp: p.Gln311*, | 1.00E−06 |
| | ENST00000319921, | p.Gln311*, | |
| | ENST00000508628 | p.Gln360* | |
| 17:80288756:G:A | ENST00000582970, | hgvsc: c.933 + 1G > A, | 3.00E−06 |
| | ENST00000319921, | c.933 + 1G > A, | |
| | ENST00000508628 | c.1080 + 1G > A | |
| 17:80289657:A:G | ENST00000582970, | hgvsc: c.934 − 2A > G, | 1.00E−06 |
| | ENST00000319921, | c.934 − 2A > G, | |
| | ENST00000508628 | c.1081 − 2A > G | |
| 17:80289698:GA:G | ENST00000582970, | hgvsp: p.Glu326fs, | 1.00E−06 |
| | ENST00000319921, | p.Glu326fs, | |
| | ENST00000508628 | p.Glu375fs | |
| 17:80289762:A:AC | ENST00000582970, | hgvsp: p.Arg347fs, | 1.00E−06 |
| | ENST00000319921, | p.Arg347fs, | |
| | ENST00000508628 | p.Arg396fs | |
| 17:80289788:G:T | ENST00000582970, | hgvsp: p.Glu355*, | 1.00E−06 |
| | ENST00000319921, | p.Glu355*, | |
| | ENST00000508628 | p.Glu404* | |
| 17:80289797:CA:C | ENST00000582970, | hgvsp: p.Asn360fs, | 1.00E−06 |
| | ENST00000319921, | p.Asn360fs, | |
| | ENST00000508628 | p.Asn409fs | |
| 17:80289820:CCAGGA:C | ENST00000582970, | hgvsp: p.Gln366fs, | 1.00E−06 |
| | ENST00000319921, | p.Gln366fs, | |
| | ENST00000508628 | p.Gln415fs | |
| 17:80289828:TG:T | ENST00000582970, | hgvsp: p.Lys369fs, | 1.00E−06 |
| | ENST00000319921, | p.Lys369fs, | |
| | ENST00000508628 | p.Lys418fs | |
| 17:80290568:A:T | ENST00000582970, | hgvsc: c.1113 − 2A > T, | 2.00E−06 |
| | ENST00000319921, | c.1113 − 2A > T, | |
| | ENST00000508628 | c.1260 − 2A > T | |
| 17:80290604:T:TA | ENST00000582970, | hgvsp: p.Phe383fs, | 1.00E−06 |
| | ENST00000319921, | p.Phe383fs, | |
| | ENST00000508628 | p.Phe432fs | |
| 17:80290651:TAA:T | ENST00000582970, | hgvsp: p.Lys399fs, | 1.00E−06 |
| | ENST00000319921, | p.Lys399fs, | |
| | ENST00000508628 | p.Lys448fs | |
| 17:80290689:C:CA | ENST00000582970, | hgvsp: p.Trp413fs, | 1.00E−06 |
| | ENST00000319921, | p.Trp413fs, | |
| | ENST00000508628 | p.Trp462fs | |
| 17:80290730:T:G | ENST00000582970, | hgvsc: c.1271 + 2T > G, | 1.00E−06 |
| | ENST00000319921, | c.1271 + 2T > G, | |
| | ENST00000508628 | c.1418 + 2T > G | |
| 17:80291626:A:G | ENST00000582970, | hgvsc: c.1272 − 2A > G, | 1.97E−06 |
| | ENST00000319921, | c.1272 − 2A > G, | |
| | ENST00000508628 | c.1419 − 2A > G | |
| 17:80291689:G:GA | ENST00000582970, | hgvsp: p.Asp445fs, | 1.00E−06 |
| | ENST00000319921, | p.Asp445fs, | |
| | ENST00000508628 | p.Asp494fs | |
| 17:80291754:C:G | ENST00000582970, | hgvsp: p.Tyr466*, | 7.35E−06 |
| | ENST00000319921, | p.Tyr466*, | |
| | ENST00000508628 | p.Tyr515* | |
| 17:80291764:C:T | ENST00000582970, | hgvsp: p.Gln470*, | 3.00E−06 |
| | ENST00000319921, | p.Gln470*, | |
| | ENST00000508628 | p.Gln519* | |
| 17:80291794:CTG:C | ENST00000582970, | hgvsp: p.Phe481fs, | 3.00E−06 |
| | ENST00000319921, | p.Phe481fs, | |
| | ENST00000508628 | p.Phe530fs | |
| 17:80291822:C:G | ENST00000582970, | hgvsp: p.Ser489*, | 4.00E−06 |
| | ENST00000319921, | p.Ser489*, | |
| | ENST00000508628 | p.Ser538* | |
| 17:80291829:T:C | ENST00000582970, | hgvsc: c.1471 + 2T > C, | 1.00E−06 |
| | ENST00000319921, | c.1471 + 2T > C, | |
| | ENST00000508628 | c.1618 + 2T > C | |
| 17:80294941:C:T | ENST00000582970, | hgvsp: p.Gln565*, | 1.00E−06 |
| | ENST00000319921, | p.Gln565*, | |
| | ENST00000508628 | p.Gln614* | |
| 17:80295004:G:A | ENST00000582970, | hgvsc: c.1755 + 1G > A, | 4.00E−06 |
| | ENST00000319921, | c.1755 + 1G > A, | |
| | ENST00000508628 | c.1902 + 1G > A | |
| 17:80295004:G:T | ENST00000582970, | hgvsc: c.1755 + 1G > T, | 1.00E−06 |
| | ENST00000319921, | c.1755 + 1G > T, | |
| | ENST00000508628 | c.1902 + 1G > T | |
| 17:80295555:A:T | ENST00000582970, | hgvsc: c.1756 − 2A > T, | 1.00E−06 |
| | ENST00000319921, | c.1756 − 2A > T, | |
| | ENST00000508628 | c.1903 − 2A > T | |
| 17:80295567:A:AC | ENST00000582970, | hgvsp: p.Leu590fs, | 1.00E−06 |
| | ENST00000319921, | p.Leu590fs, | |
| | ENST00000508628 | p.Leu639fs | |
| 17:80295568:C:G | ENST00000582970, | hgvsp: p.Tyr589*, | 8.00E−06 |
| | ENST00000319921, | p.Tyr589*, | |
| | ENST00000508628 | p.Tyr638* | |
| 17:80295741:TC:T | ENST00000582970, | hgvsp: p.Leu648fs, | 1.00E−06 |
| | ENST00000319921, | p.Leu648fs, | |
| | ENST00000508628 | p.Leu697fs | |
| 17:80295808:T:TC | ENST00000582970, | hgvsp: p.Gln670fs, | 1.96E−06 |
| | ENST00000319921, | p.Gln670fs, | |
| | ENST00000508628 | p.Gln719fs | |
| 17:80295813:GGTATTAT:G | ENST00000582970, | hgvsc: | 1.00E−06 |
| | ENST00000319921, | c.2012 + 1_2012 + 7delGTATTAT, | |
| | ENST00000508628 | c.2012 + 1_2012 + 7delGTATTAT, | |
| | | c.2159 + 1_2159 + 7delGTATTAT | |
| 17:80295814:GTAT:G | ENST00000582970, | hgvsc: | 3.57E−06 |
| | ENST00000319921, | c.2012 + 2_2012 + 4delTAT, | |
| | ENST00000508628 | c.2012 + 2_2012 + 4delTAT, | |
| | | c.2159 + 2_2159 + 4delTAT | |
| 17:80295814:G:A | ENST00000582970, | hgvsc: c.2012 + 1G > A, | 1.00E−06 |
| | ENST00000319921, | c.2012 + 1G > A, | |
| | ENST00000508628 | c.2159 + 1G > A | |
| 17:80298410:G:GTA | ENST00000582970, | hgvsp: p.Met702fs, | 1.00E−06 |
| | ENST00000319921, | p.Met702fs, | |
| | ENST00000508628 | p.Met751fs | |
| 17:80298469:G:GC | ENST00000582970, | hgvsp: p.Leu722fs, | 1.00E−06 |
| | ENST00000319921, | p.Leu722fs, | |
| | ENST00000508628 | p.Leu771fs | |
| 17:80306283:C:T | ENST00000582970, | hgvsp: p.Gln748*, | 7.00E−06 |
| | ENST00000319921, | p.Gln748*, | |
| | ENST00000508628 | p.Gln797* | |
| 17:80306419:C:G | ENST00000582970, | hgvsp: p.Ser793*, | 1.00E−06 |
| | ENST00000319921, | p.Ser793*, | |
| | ENST00000508628 | p.Ser842* | |
| 17:80307152:C:T | ENST00000582970, | hgvsp: p.Gln818*, | 1.00E−06 |
| | ENST00000319921, | p.Gln818*, | |
| | ENST00000508628 | p.Gln867* | |
| 17:80307176:C:T | ENST00000582970, | hgvsp: p.Arg826*, | 1.88E−06 |
| | ENST00000319921, | p.Arg826*, | |
| | ENST00000508628 | p.Arg875* | |
| 17:80307189:CT:C | ENST00000582970, | hgvsp: p.Tyr831fs, | 4.00E−06 |
| | ENST00000319921, | p.Tyr831fs, | |
| | ENST00000508628 | p.Tyr880fs | |
| 17:80309053:CTGTG:C | ENST00000582970, | hgvsp: p.Cys848fs, | 1.00E−06 |
| | ENST00000319921, | p.Cys848fs, | |
| | ENST00000508628 | p.Cys897fs | |
| 17:80313011:G:C | ENST00000582970, | hgvsc: c.2656 − 1G > C, | 1.00E−06 |
| | ENST00000319921, | c.2656 − 1G > C, | |
| | ENST00000508628 | c.2803 − 1G > C | |
| 17:80313024:CAG:C | ENST00000582970, | hgvsp: p.Asp892fs, | 2.91E−06 |
| | ENST00000319921, | p.Asp892fs, | |
| | ENST00000508628 | p.Asp941fs | |
| 17:80313082:C:CT | ENST00000582970, | hgvsp: p.Thr910fs, | 1.00E−06 |
| | ENST00000319921, | p.Thr910fs, | |
| | ENST00000508628 | p.Thr959fs | |
| 17:80313091:GGT:G | ENST00000582970, | hgvsp: p.Trp913fs, | 1.00E−06 |
| | ENST00000319921, | p.Trp913fs, | |
| | ENST00000508628 | p.Trp962fs | |
| 17:80313146:CACAT:C | ENST00000582970, | hgvsp: p.Tyr932fs, | 3.71E−06 |
| | ENST00000319921, | p.Tyr932fs, | |
| | ENST00000508628 | p.Tyr981fs | |
| 17:80317278:G:A | ENST00000582970, | hgvsc: c.2901 + 1G > A, | 1.00E−06 |
| | ENST00000319921, | c.2901 + 1G > A, | |
| | ENST00000508628 | c.3048 + 1G > A | |
| 17:80319373:C:T | ENST00000319921 | hgvsp: p.Gln1029* | 1.06E−05 |
| 17:80319422:CAG:C | ENST00000319921 | hgvsp: p.Glu1046fs | 2.00E−06 |
| 17:80319437:G:A | ENST00000319921 | hgvsp: p.Trp1050* | 1.00E−06 |
| 17:80325108:A:T | ENST00000582970, | hgvsp: p.Lys1035*, | 1.00E−06 |
| | ENST00000508628 | p.Lys1084* | |
| 17:80325116:GC:G | ENST00000582970, | hgvsp: p.Pro1038fs, | 1.00E−06 |
| | ENST00000508628 | p.Pro1087fs | |
| 17:80325166:T:A | ENST00000582970, | hgvsp: p.Leu1054*, | 5.00E−06 |
| | ENST00000508628 | p.Leu1103* | |
| 17:80325199:GT:G | ENST00000582970, | hgvsc: c.3193 + 2delT, | 1.00E−06 |
| | ENST00000508628 | c.3340 + 2delT | |
| 17:80327879:ACT:A | ENST00000582970, | hgvsp: p.Ser1087fs, | 1.00E−06 |
| | ENST00000508628 | p.Ser1136fs | |
| 17:80327967:GT:G | ENST00000582970, | hgvsp: p.Asp1118fs, | 4.00E−06 |
| | ENST00000508628 | p.Asp1167fs | |
| 17:80327981:G:A | ENST00000582970, | hgvsp: p.Trp1120*, | 1.00E−06 |
| | ENST00000508628 | p.Trp1169* | |
| 17:80327991:T:C | ENST00000582970, | hgvsc: c.3367 + 2T > C, | 3.00E−06 |
| | ENST00000508628 | c.3514 + 2T > C | |
| 17:80328326:A:G | ENST00000582970, | hgvsc: c.3368 − 2A > G, | 1.00E−06 |
| | ENST00000508628 | c.3515 − 2A > G | |
| 17:80328330:G:GA | ENST00000582970, | hgvsp: p.Ser1126fs, | 2.00E−06 |
| | ENST00000508628 | p.Ser1175fs | |
| 17:80328330:G:T | ENST00000582970, | hgvsp: p.Glu1124*, | 1.00E−06 |
| | ENST00000508628 | p.Glu1173* | |
| 17:80328396:G:T | ENST00000582970, | hgvsp: p.Glu1146*, | 1.00E−06 |
| | ENST00000508628 | p.Glu1195* | |
| 17:80328427:GT:G | ENST00000582970, | hgvsp: p.Cys1156fs, | 3.00E−06 |
| | ENST00000508628 | p.Cys1205fs | |
| 17:80328473:AC:A | ENST00000582970, | hgvsp: p.Gln1172fs, | 2.00E−06 |
| | ENST00000508628 | p.Gln1221fs | |
| 17:80332004:A:G | ENST00000582970, | hgvsc: c.3518 − 2A > G, | 1.00E−06 |
| | ENST00000508628 | c.3665 − 2A > G | |
| 17:80332005:G:A | ENST00000582970, | hgvsc: c.3518 − 1G > A, | 1.00E−06 |
| | ENST00000508628 | c.3665 − 1G > A | |
| 17:80332012:TTG:T | ENST00000582970, | hgvsp: p.Phe1175fs, | 1.00E−06 |
| | ENST00000508628 | p.Phe1224fs | |
| 17:80332036:CA:C | ENST00000582970, | hgvsp: p.Gln1184fs, | 1.00E−06 |
| | ENST00000508628 | p.Gln1233fs | |
| 17:80332038:C:T | ENST00000582970, | hgvsp: p.Gln1184*, | 1.00E−06 |
| | ENST00000508628 | p.Gln1233* | |
| 17:80332188:C:T | ENST00000582970, | hgvsp: p.Gln1234*, | 1.88E−06 |
| | ENST00000508628 | p.Gln1283* | |
| 17:80332224:GA:G | ENST00000582970, | hgvsp: p.Glu1246fs, | 1.00E−06 |
| | ENST00000508628 | p.Glu1295fs | |
| 17:80332231:A:AG | ENST00000582970, | hgvsp: p.Glu1249fs, | 1.00E−06 |
| | ENST00000508628 | p.Glu1298fs | |
| 17:80332250:AGAGTTGCT:A | ENST00000582970, | hgvsp: p.Leu1256fs, | 1.00E−06 |
| | ENST00000508628 | p.Leu1305fs | |
| 17:80332302:GA:G | ENST00000582970, | hgvsp: p.Glu1273fs, | 4.00E−06 |
| | ENST00000508628 | p.Glu1322fs | |
| 17:80332631:TG:T | ENST00000582970, | hgvsc: c.4143 + 1delG, | 1.00E−06 |
| | ENST00000508628 | c.4290 + 1delG | |
| 17:80332632:G:A | ENST00000582970, | hgvsc: c.4143 + 1G > A, | 1.00E−06 |
| | ENST00000508628 | c.4290 + 1G > A | |
| 17:80336159:A:C | ENST00000582970, | hgvsc: c.4310 − 2A > C, | 5.00E−06 |
| | ENST00000508628 | c.4457 − 2A > C | |
| 17:80337615:GAA:G | ENST00000582970, | hgvsp: p.Lys1520fs, | 1.00E−06 |
| | ENST00000508628 | p.Lys1569fs | |
| 17:80337636:TG:T | ENST00000582970, | hgvsp: p.Ser1528fs, | 2.00E−06 |
| | ENST00000508628 | p.Ser1577fs | |
| 17:80337832:G:A | ENST00000582970, | hgvsc: c.4669 − 1G > A, | 2.00E−06 |
| | ENST00000508628 | c.4816 − 1G > A | |
| 17:80337896:C:T | ENST00000582970, | hgvsp: p.Arg1578*, | 2.00E−06 |
| | ENST00000508628 | p.Arg1627* | |
| 17:80337977:G:T | ENST00000582970, | hgvsp: p.Glu1605*, | 1.00E−06 |
| | ENST00000508628 | p.Glu1654* | |
| 17:80339278:G:A | ENST00000582970, | hgvsp: p.Trp1637*, | 8.86E−06 |
| | ENST00000508628 | p.Trp1686* | |
| 17:80339450:C:T | ENST00000582970, | hgvsp: p.Arg1695*, | 2.09E−05 |
| | ENST00000508628 | p.Arg1744* | |
| 17:80339524:GC:G | ENST00000582970, | hgvsp: p.Pro1721fs, | 4.62E−06 |
| | ENST00000508628 | p.Pro1770fs | |
| 17:80339592:TA:T | ENST00000582970, | hgvsp: p.Arg1743fs, | 2.00E−06 |
| | ENST00000508628 | p.Arg1792fs | |
| 17:80339594:AG:A | ENST00000582970, | hgvsp: p.Ala1744fs, | 1.00E−06 |
| | ENST00000508628 | p.Ala1793fs | |
| 17:80339602:T:TG | ENST00000582970, | hgvsp: p.Val1746fs, | 1.00E−06 |
| | ENST00000508628 | p.Val1795fs | |
| 17:80339632:C:G | ENST00000582970, | hgvsp: p.Tyr1755*, | 1.00E−06 |
| | ENST00000508628 | p.Tyr1804* | |
| 17:80339654:G:T | ENST00000582970, | hgvsp: p.Glu1763*, | 1.00E−06 |
| | ENST00000508628 | p.Glu1812* | |
| 17:80339673:T:TTG | ENST00000582970, | hgvsp: p.Leu1769fs, | 2.00E−06 |
| | ENST00000508628 | p.Leu1818fs | |
| 17:80339728:G:GTGCCTTCC | ENST00000582970, | hgvsp: p.Asp1795fs, | 3.93E−06 |
| | ENST00000508628 | p.Asp1844fs | |
| 17:80339728:GTGCCTTCC:G | ENST00000582970, | hgvsp: p.Phe1792fs, | 1.00E−05 |
| | ENST00000508628 | p.Phe1841fs | |
| 17:80339741:T:TTCCTGCCC | ENST00000582970, | hgvsp: p.Asp1795fs, | 1.00E−06 |
| | ENST00000508628 | p.Asp1844fs | |
| 17:80339894:GC:G | ENST00000582970, | hgvsp: p.Ala1844fs, | 1.00E−06 |
| | ENST00000508628 | p.Ala1893fs | |
| 17:80339942:T:TA | ENST00000582970, | hgvsp: p.Tyr1859fs, | 8.00E−06 |
| | ENST00000508628 | p.Tyr1908fs | |
| 17:80340010:C:A | ENST00000582970, | hgvsp: p.Cys1881*, | 1.00E−06 |
| | ENST00000508628 | p.Cys1930* | |
| 17:80340110:CTG:C | ENST00000582970, | hgvsp: p.Cys1916fs, | 1.40E−05 |
| | ENST00000508628 | p.Cys1965fs | |
| 17:80340169:G:A | ENST00000582970, | hgvsp: p.Trp1934*, | 3.00E−06 |
| | ENST00000508628 | p.Trp1983* | |
| 17:80340253:C:A | ENST00000582970, | hgvsp: p.Tyr1962*, | 5.00E−06 |
| | ENST00000508628 | p.Tyr2011* | |
| 17:80340258:CA:C | ENST00000582970, | hgvsp: p.Gly1965fs, | 1.00E−06 |
| | ENST00000508628 | p.Gly2014fs | |
| 17:80340357:G:A | ENST00000582970, | hgvsc: c.5989 + 1G > A, | 1.00E−06 |
| | ENST00000508628 | c.6136 + 1G > A | |
| 17:80343267:TC:T | ENST00000582970, | hgvsp: p.Leu2043fs, | 1.00E−06 |
| | ENST00000508628 | p.Leu2092fs | |
| 17:80343278:C:T | ENST00000582970, | hgvsp: p.Gln2046*, | 1.00E−06 |
| | ENST00000508628 | p.Gln2095* | |
| 17:80343284:C:T | ENST00000582970, | hgvsp: p.Gln2048*, | 1.00E−06 |
| | ENST00000508628 | p.Gln2097* | |
| 17:80343286:GA:G | ENST00000582970, | hgvsp: p.Lys2049fs, | 1.00E−06 |
| | ENST00000508628 | p.Lys2098fs | |
| 17:80343297:TG:T | ENST00000582970, | hgvsp: p.Leu2053fs, | 1.00E−06 |
| | ENST00000508628 | p.Leu2102fs | |
| 17:80343856:G:T | ENST00000582970, | hgvsc: c.6184 − 1G > T, | 1.00E−06 |
| | ENST00000508628 | c.6331 − 1G > T | |
| 17:80343880:TC:T | ENST00000582970, | hgvsp: p.Leu2070fs, | 3.00E−06 |
| | ENST00000508628 | p.Leu2119fs | |
| 17:80343902:C:T | ENST00000582970, | hgvsp: p.Gln2077*, | 1.00E−06 |
| | ENST00000508628 | p.Gln2126* | |
| 17:80343907:C:G | ENST00000582970, | hgvsp: p.Tyr2078*, | 1.00E−06 |
| | ENST00000508628 | p.Tyr2127* | |
| 17:80343933:G:A | ENST00000582970, | hgvsp: p.Trp2087*, | 3.10E−05 |
| | ENST00000508628 | p.Trp2136* | |
| 17:80343951:AT:A | ENST00000582970, | hgvsp: p.Leu2094fs, | 1.88E−06 |
| | ENST00000508628 | p.Leu2143fs | |
| 17:80343953:TTGTA:T | ENST00000582970, | hgvsp: p.Tyr2095fs, | 2.00E−06 |
| | ENST00000508628 | p.Tyr2144fs | |
| 17:80344008:C:A | ENST00000582970, | hgvsp: p.Ser2112*, | 1.00E−06 |
| | ENST00000508628 | p.Ser2161* | |
| 17:80344695:G:GTT | ENST00000582970, | hgvsp: p.Phe2123fs, | 2.00E−06 |
| | ENST00000508628 | p.Phe2172fs | |
| 17:80344718:CA:C | ENST00000582970, | hgvsp: p.Val2130fs, | 1.00E−06 |
| | ENST00000508628 | p.Val2179fs | |
| 17:80344815:CA:C | ENST00000582970, | hgvsp: p.Ser2161fs, | 1.00E−06 |
| | ENST00000508628 | p.Ser2210fs | |
| 17:80344839:C:A | ENST00000582970, | hgvsp: p.Tyr2168*, | 3.00E−06 |
| | ENST00000508628 | p.Tyr2217* | |
| 17:80344950:C:A | ENST00000582970, | hgvsp: p.Cys2205*, | 1.00E−06 |
| | ENST00000508628 | p.Cys2254* | |
| 17:80344950:C:CG | ENST00000582970, | hgvsp: p.Val2207fs, | 1.00E−06 |
| | ENST00000508628 | p.Val2256fs | |
| 17:80344961:AC:A | ENST00000582970, | hgvsp: p.Pro2210fs, | 1.00E−06 |
| | ENST00000508628 | p.Pro2259fs | |
| 17:80345037:CT:C | ENST00000582970, | hgvsp: p.Cys2235fs, | 3.83E−06 |
| | ENST00000508628 | p.Cys2284fs | |
| 17:80345064:A:AC | ENST00000582970, | hgvsp: p.Leu2244fs, | 2.00E−06 |
| | ENST00000508628 | p.Leu2293fs | |
| 17:80345066:TGA:T | ENST00000582970, | hgvsp: p.Arg2245fs, | 3.71E−06 |
| | ENST00000508628 | p.Arg2294fs | |
| 17:80345250:C:A | ENST00000582970, | hgvsp: p.Tyr2305*, | 3.00E−06 |
| | ENST00000508628 | p.Tyr2354* | |
| 17:80345302:C:T | ENST00000582970, | hgvsp: p.Gln2323*, | 1.00E−06 |
| | ENST00000508628 | p.Gln2372* | |
| 17:80345359:AAG:A | ENST00000582970, | hgvsp: p.Asp2344fs, | 1.00E−06 |
| | ENST00000508628 | p.Asp2393fs | |
| 17:80345388:C:G | ENST00000582970, | hgvsp: p.Tyr2351*, | 1.00E−06 |
| | ENST00000508628 | p.Tyr2400* | |
| 17:80345458:G:T | ENST00000582970, | hgvsp: p.Glu2375*, | 4.00E−06 |
| | ENST00000508628 | p.Glu2424* | |
| 17:80345483:T:TC | ENST00000582970, | hgvsp: p.Gln2385fs, | 1.00E−06 |
| | ENST00000508628 | p.Gln2434fs | |
| 17:80345488:C:T | ENST00000582970, | hgvsp: p.Gln2385*, | 9.00E−06 |
| | ENST00000508628 | p.Gln2434* | |
| 17:80345797:G:T | ENST00000582970, | hgvsp: p.Glu2488*, | 1.00E−06 |
| | ENST00000508628 | p.Glu2537* | |
| 17:80345881:T:TC | ENST00000582970, | hgvsp: p.Gly2517fs, | 1.00E−06 |
| | ENST00000508628 | p.Gly2566fs | |
| 17:80345974:G:GT | ENST00000582970, | hgvsp: p.Ser2548fs, | 1.00E−06 |
| | ENST00000508628 | p.Ser2597fs | |
| 17:80346095:AC:A | ENST00000582970, | hgvsp: p.Asp2587fs, | 1.00E−06 |
| | ENST00000508628 | p.Asp2636fs | |
| 17:80346270:GAA:G | ENST00000582970, | hgvsp: p.Lys2646fs, | 1.88E−06 |
| | ENST00000508628 | p.Lys2695fs | |
| 17:80346351:CA:C | ENST00000582970, | hgvsp: p.Asn2674fs, | 3.00E−06 |
| | ENST00000508628 | p.Asn2723fs | |
| 17:80346384:G:A | ENST00000582970, | hgvsp: p.Trp2683*, | 3.00E−06 |
| | ENST00000508628 | p.Trp2732* | |
| 17:80346414:C:G | ENST00000582970, | hgvsp: p.Tyr2693*, | 1.00E−06 |
| | ENST00000508628 | p.Tyr2742* | |
| 17:80346455:TC:T | ENST00000582970, | hgvsp: p.Ile2707fs, | 1.00E−06 |
| | ENST00000508628 | p.Ile2756fs | |
| 17:80346484:GA:G | ENST00000582970, | hgvsp: p.Asp2717fs, | 3.00E−06 |
| | ENST00000508628 | p.Asp2766fs | |
| 17:80346624:TC:T | ENST00000582970, | hgvsp: p.Leu2765fs, | 1.00E−06 |
| | ENST00000508628 | p.Leu2814fs | |
| 17:80346632:TC:T | ENST00000582970, | hgvsp: p.Leu2767fs, | 1.00E−06 |
| | ENST00000508628 | p.Leu2816fs | |
| 17:80346781:CAG:C | ENST00000582970, | hgvsp: p.Gln2816fs, | 1.00E−06 |
| | ENST00000508628 | p.Gln2865fs | |
| 17:80346820:C:T | ENST00000582970, | hgvsp: p.Gln2829*, | 2.00E−06 |
| | ENST00000508628 | p.Gln2878* | |
| 17:80346846:C:A | ENST00000582970, | hgvsp: p.Tyr2837*, | 1.00E−06 |
| | ENST00000508628 | p.Tyr2886* | |
| 17:80346955:G:GC | ENST00000582970, | hgvsp: p.His2876fs, | 5.00E−06 |
| | ENST00000508628 | p.His2925fs | |
| 17:80347044:CA:C | ENST00000582970, | hgvsp: p.Ser2904fs, | 1.00E−06 |
| | ENST00000508628 | p.Ser2953fs | |
| 17:80347171:C:T | ENST00000582970, | hgvsp: p.Gln2946*, | 1.00E−06 |
| | ENST00000508628 | p.Gln2995* | |
| 17:80347178:AG:A | ENST00000582970, | hgvsp: p.Glu2949fs, | 1.00E−06 |
| | ENST00000508628 | p.Glu2998fs | |
| 17:80347180:G:T | ENST00000582970, | hgvsp: p.Glu2949*, | 1.00E−06 |
| | ENST00000508628 | p.Glu2998* | |
| 17:80347393:CAG:C | ENST00000582970, | hgvsp: p.Asn3021fs, | 2.00E−06 |
| | ENST00000508628 | p.Asn3070fs | |
| 17:80347435:C:T | ENST00000582970, | hgvsp: p.Gln3034*, | 2.00E−06 |
| | ENST00000508628 | p.Gln3083* | |
| 17:80347460:T:G | ENST00000582970, | hgvsp: p.Leu3042*, | 4.00E−06 |
| | ENST00000508628 | p.Leu3091* | |
| 17:80347573:TAC:T | ENST00000582970, | hgvsp: p.Thr3081fs, | 1.00E−06 |
| | ENST00000508628 | p.Thr3130fs | |
| 17:80347684:A:AGG | ENST00000582970, | hgvsp: p.Asn3117fs, | 1.00E−06 |
| | ENST00000508628 | p.Asn3166fs | |
| 17:80347689:G:GGTCC | ENST00000582970, | hgvsp: p.Tyr3119fs, | 1.00E−06 |
| | ENST00000508628 | p.Tyr3168fs | |
| 17:80347690:TA:T | ENST00000582970, | hgvsp: p.Tyr3119fs, | 1.00E−06 |
| | ENST00000508628 | p.Tyr3168fs | |
| 17:80347695:C:G | ENST00000582970, | hgvsp: p.Tyr3120*, | 1.00E−06 |
| | ENST00000508628 | p.Tyr3169* | |
| 17:80347807:TACAA:T | ENST00000582970, | hgvsp: p.Lys3159fs, | 1.00E−06 |
| | ENST00000508628 | p.Lys3208fs | |
| 17:80347815:CT:C | ENST00000582970, | hgvsp: p.Ile3163fs, | 1.00E−06 |
| | ENST00000508628 | p.Ile3212fs | |
| 17:80347884:G:A | ENST00000582970, | hgvsp: p.Trp3183*, | 2.00E−06 |
| | ENST00000508628 | p.Trp3232* | |
| 17:80347998:C:G | ENST00000582970, | hgvsp: p.Tyr3221*, | 1.00E−06 |
| | ENST00000508628 | p.Tyr3270* | |
| 17:80348105:TC:T | ENST00000582970, | hgvsp: p.Leu3258fs, | 1.00E−06 |
| | ENST00000508628 | p.Leu3307fs | |
| 17:80348180:G:A | ENST00000582970, | hgvsp: p.Trp3282*, | 2.00E−06 |
| | ENST00000508628 | p.Trp3331* | |
| 17:80348181:G:A | ENST00000582970, | hgvsp: p.Trp3282*, | 1.00E−06 |
| | ENST00000508628 | p.Trp3331* | |
| 17:80348196:C:A | ENST00000582970, | hgvsp: p.Tyr3287*, | 9.00E−06 |
| | ENST00000508628 | p.Tyr3336* | |
| 17:80349779:TTC:T | ENST00000582970, | hgvsp: p.Ser3322fs, | 1.00E−06 |
| | ENST00000508628 | p.Ser3371fs | |
| 17:80349905:C:T | ENST00000582970, | hgvsp: p.Arg3363*, | 9.00E−06 |
| | ENST00000508628 | p.Arg3412* | |
| 17:80349907:G:T | ENST00000582970, | hgvsc: c.10088 + 1G > T, | 1.00E−06 |
| | ENST00000508628 | c.10235 + 1G > T | |
| 17:80350338:A:AT | ENST00000582970, | hgvsp: p.Gln3378fs, | 1.00E−06 |
| | ENST00000508628 | p.Gln3427fs | |
| 17:80350377:C:T | ENST00000582970, | hgvsp: p.Gln3389*, | 1.00E−06 |
| | ENST00000508628 | p.Gln3438* | |
| 17:80350398:T:C | ENST00000582970, | hgvsc: c.10184 + 2T > C, | 1.00E−06 |
| | ENST00000508628 | c.10331 + 2T > C | |
| 17:80351684:G:A | ENST00000582970, | hgvsc: c.10185 − 1G > A, | 1.00E−06 |
| | ENST00000508628 | c.10332 − 1G > A | |
| 17:80352939:G:T | ENST00000582970, | hgvsc: c.10304 − 1G > T, | 1.96E−06 |
| | ENST00000508628 | c.10451 − 1G > T | |
| 17:80353029:C:T | ENST00000582970, | hgvsp: p.Gln3465*, | 2.00E−06 |
| | ENST00000508628 | p.Gln3514* | |
| 17:80353510:A:G | ENST00000582970, | hgvsc: c.10424 − 2A > G, | 1.00E−06 |
| | ENST00000508628 | c.10571 − 2A > G | |
| 17:80353510:A:C | ENST00000582970, | hgvsc: c.10424 − 2A > C, | 1.00E−06 |
| | ENST00000508628 | c.10571 − 2A > C | |
| 17:80354036:C:CA | ENST00000582970, | hgvsp: p.Thr3533fs, | 5.00E−06 |
| | ENST00000508628 | p.Thr3582fs | |
| 17:80354094:G:T | ENST00000582970, | hgvsp: p.Glu3552*, | 1.00E−06 |
| | ENST00000508628 | p.Glu3601* | |
| 17:80354483:T:A | ENST00000582970, | hgvsp: p.Leu3590*, | 2.91E−06 |
| | ENST00000508628 | p.Leu3639* | |
| 17:80354494:G:T | ENST00000582970, | hgvsp: p.Glu3594*, | 1.00E−06 |
| | ENST00000508628 | p.Glu3643* | |
| 17:80354495:AAG:A | ENST00000582970, | hgvsp: p.Ser3596fs, | 1.00E−06 |
| | ENST00000508628 | p.Ser3645fs | |
| 17:80354557:C:T | ENST00000582970, | hgvsp: p.Gln3615*, | 1.00E−06 |
| | ENST00000508628 | p.Gln3664* | |
| 17:80354575:AGGT:A | ENST00000582970, | hgvsp: p.Arg3621fs, | 1.00E−05 |
| | ENST00000508628 | p.Arg3670fs | |
| 17:80358299:G:A | ENST00000582970, | hgvsp: p.Trp3625*, | 1.00E−06 |
| | ENST00000508628 | p.Trp3674* | |
| 17:80358310:C:T | ENST00000582970, | hgvsp: p.Gln3629*, | 3.00E−06 |
| | ENST00000508628 | p.Gln3678* | |
| 17:80358357:CAG:C | ENST00000582970, | hgvsp: p.Asp3646fs, | 2.00E−06 |
| | ENST00000508628 | p.Asp3695fs | |
| 17:80358357:C:CA | ENST00000582970, | hgvsp: p.Arg3645fs, | 5.00E−06 |
| | ENST00000508628 | p.Arg3694fs | |
| 17:80358404:G:A | ENST00000582970, | hgvsp: p.Trp3660*, | 1.00E−06 |
| | ENST00000508628 | p.Trp3709* | |
| 17:80358438:C:CA | ENST00000582970, | hgvsp: p.Thr3672fs, | 2.00E−06 |
| | ENST00000508628 | p.Thr3721fs | |
| 17:80361759:C:CA | ENST00000582970, | hgvsp: p.Phe3743fs, | 3.00E−06 |
| | ENST00000508628 | p.Phe3792fs | |
| 17:80363098:CCA:C | ENST00000582970, | hgvsc: c.H356 − 3_11356 − 2delCA, | 7.00E−06 |
| | ENST00000508628 | c.H503 − 3_11503 − 2delCA | |
| 17:80363151:C:G | ENST00000582970, | hgvsp: p.Ser3802*, | 1.30E−05 |
| | ENST00000508628 | p.Ser3851* | |
| 17:80363158:GC:G | ENST00000582970, | hgvsp: p.Glu3806fs, | 8.00E−06 |
| | ENST00000508628 | p.Glu3855fs | |
| 17:80363185:G:A | ENST00000582970, | hgvsp: p.Trp3813*, | 1.00E−06 |
| | ENST00000508628 | p.Trp3862* | |
| 17:80363199:A:AG | ENST00000582970, | hgvsp: p.Tyr3818fs, | 2.00E−06 |
| | ENST00000508628 | p.Tyr3867fs | |
| 17:80363200:C:A | ENST00000582970, | hgvsp: p.Tyr3818*, | 2.00E−06 |
| | ENST00000508628 | p.Tyr3867* | |
| 17:80363608:G:A | ENST00000582970, | hgvsc: c.11569 − 1G > A, | 1.00E−06 |
| | ENST00000508628 | c.11716 − 1G > A | |
| 17:80363674:TC:T | ENST00000582970, | hgvsp: p.Gln3880fs, | 1.00E−06 |
| | ENST00000508628 | p.Gln3929fs | |
| 17:80363780:AG:A | ENST00000582970, | hgvsp: p.Glu3915fs, | 3.00E−06 |
| | ENST00000508628 | p.Glu3964fs | |
| 17:80364441:G:A | ENST00000582970, | hgvsp: p.Trp3920*, | 1.88E−06 |
| | ENST00000508628 | p.Trp3969* | |
| 17:80364539:TTCTTACTA:T | ENST00000582970, | hgvsp: p.Phe3953fs, | 1.00E−06 |
| | ENST00000508628 | p.Phe4002fs | |
| 17:80367764:C:CT | ENST00000582970, | hgvsp: p.Asp3964fs, | 1.00E−06 |
| | ENST00000508628 | p.Asp4013fs | |
| 17:80367765:T:TGACGTGAA | ENST00000582970, | hgvsp: p.His3968fs, | 1.00E−06 |
| | ENST00000508628 | p.His4017fs | |
| 17:80368024:C:A | ENST00000582970, | hgvsp: p.Cys4012*, | 1.00E−06 |
| | ENST00000508628 | p.Cys4061* | |
| 17:80368059:G:A | ENST00000582970, | hgvsp: p.Trp4024*, | 2.00E−06 |
| | ENST00000508628 | p.Trp4073* | |
| 17:80368090:C:A | ENST00000582970, | hgvsp: p.Tyr4034*, | 1.00E−06 |
| | ENST00000508628 | p.Tyr4083* | |
| 17:80369513:AAAAG:A | ENST00000582970, | hgvsp: p.Glu4056fs, | 1.00E−06 |
| | ENST00000508628 | p.Glu4105fs | |
| 17:80369542:AAC:A | ENST00000582970, | hgvsp: p.Asn4066fs, | 2.90E−06 |
| | ENST00000508628 | p.Asn4115fs | |
| 17:80369595:GC:G | ENST00000582970, | hgvsp: p.Pro4084fs, | 1.00E−06 |
| | ENST00000508628 | p.Pro4133fs | |
| 17:80369668:CA:C | ENST00000582970, | hgvsp: p.Gln4108fs, | 3.00E−06 |
| | ENST00000508628 | p.Gln4157fs | |
| 17:80369672:G:A | ENST00000582970, | hgvsc: c.12325 + 1G > A, | 4.00E−06 |
| | ENST00000508628 | c.12472 + 1G > A | |
| 17:80369673:T:G | ENST00000582970, | hgvsc: c.12325 + 2T > G, | 2.00E−06 |
| | ENST00000508628 | c.12472 + 2T > G | |
| 17:80369673:T:C | ENST00000582970, | hgvsc: c.12325 + 2T > C, | 3.00E−06 |
| | ENST00000508628 | c.12472 + 2T > C | |
| 17:80369787:ATC:A | ENST00000582970, | hgvsp: p.Pro4119fs, | 5.53E−06 |
| | ENST00000508628 | p.Pro4168fs | |
| 17:80369787:ATCTC:A | ENST00000582970, | hgvsp: p.Ser4118fs, | 2.00E−06 |
| | ENST00000508628 | p.Ser4167fs | |
| 17:80369869:T:A | ENST00000582970, | hgvsc: c.12425 + 2T > A, | 1.00E−06 |
| | ENST00000508628 | c.12572 + 2T > A | |
| 17:80371872:A:G | ENST00000582970, | hgvsc: c.12426 − 2A > G, | 1.88E−06 |
| | ENST00000508628 | c.12573 − 2A > G | |
| 17:80371887:AAAGATTAT:A | ENST00000582970, | hgvsp: p.Lys4147fs, | 1.00E−06 |
| | ENST00000508628 | p.Lys4196fs | |
| 17:80371899:C:T | ENST00000582970, | hgvsp: p.Gln4151*, | 1.00E−06 |
| | ENST00000508628 | p.Gln4200* | |
| 17:80371909:T:A | ENST00000582970, | hgvsp: p.Leu4154*, | 0 |
| | ENST00000508628 | p.Leu4203* | |
| 17:80371918:T:TA | ENST00000582970, | hgvsp: p.Lys4160fs, | 3.00E−06 |
| | ENST00000508628 | p.Lys4209fs | |
| 17:80371918:TAA:T | ENST00000582970, | hgvsp: p.Lys4159fs, | 0 |
| | ENST00000508628 | p.Lys4208fs | |
| 17:80371926:A:AAAGC | ENST00000582970, | hgvsp: p.Phe4162fs, | 2.00E−06 |
| | ENST00000508628 | p.Phe4211fs | |
| 17:80371929:GCATT:G | ENST00000582970, | hgvsp: p.Phe4162fs, | 1.00E−06 |
| | ENST00000508628 | p.Phe4211fs | |
| 17:80371945:AT:A | ENST00000582970, | hgvsp: p.Asp4166fs, | 2.00E−06 |
| | ENST00000508628 | p.Asp4215fs | |
| 17:80371946:TA:T | ENST00000582970, | hgvsp: p.Thr4168fs, | 0 |
| | ENST00000508628 | p.Thr4217fs | |
| 17:80371953:G:GAACT | ENST00000582970, | hgvsp: p.Tyr4171fs, | 1.00E−06 |
| | ENST00000508628 | p.Tyr4220fs | |
| 17:80371967:CT:C | ENST00000582970, | hgvsp: p.Phe4174fs, | 1.00E−06 |
| | ENST00000508628 | p.Phe4223fs | |
| 17:80371979:C:A | ENST00000582970, | hgvsp: p.Cys4177*, | 1.00E−06 |
| | ENST00000508628 | p.Cys4226* | |
| 17:80371981:TG:T | ENST00000582970, | hgvsp: p.Glu4179fs, | 0 |
| | ENST00000508628 | p.Glu4228fs | |
| 17:80371986:GT:G | ENST00000582970, | hgvsc: c.12537 + 2delT, | 1.00E−06 |
| | ENST00000508628 | c.12684 + 2delT | |
| 17:80371986:G:GT | ENST00000582970, | hgvsc: c.12537 + 1_12537 + 2insT, | 1.00E−06 |
| | ENST00000508628 | c.12684 + 1_12684 + 2insT | |
| 17:80372525:C:G | ENST00000582970, | hgvsp: p.Ser4181*, | 4.00E−06 |
| | ENST00000508628 | p.Ser4230* | |
| 17:80372525:C:A | ENST00000582970, | hgvsp: p.Ser4181*, | 1.00E−06 |
| | ENST00000508628 | p.Ser4230* | |
| 17:80372553:CAG:C | ENST00000582970, | hgvsp: p.Arg4191fs, | 1.00E−06 |
| | ENST00000508628 | p.Arg4240fs | |
| 17:80372607:T:A | ENST00000582970, | hgvsp: p.Tyr4208*, | 4.00E−06 |
| | ENST00000508628 | p.Tyr4257* | |
| 17:80372662:C:T | ENST00000582970, | hgvsp: p.Gln4227*, | 1.00E−06 |
| | ENST00000508628 | p.Gln4276* | |
| 17:80372736:C:T | ENST00000582970, | hgvsc: c.12751 + 2C > T, | 2.00E−06 |
| | ENST00000508628 | c.12898 + 2C > T | |
| 17:80372992:C:T | ENST00000582970, | hgvsp: p.Gln4257*, | 1.00E−06 |
| | ENST00000508628 | p.Gln4306* | |
| 17:80373059:TG:T | ENST00000582970, | hgvsp: p.Val4280fs, | 3.00E−06 |
| | ENST00000508628 | p.Val4329fs | |
| 17:80373135:G:A | ENST00000582970, | hgvsp: p.Trp4304*, | 1.00E−06 |
| | ENST00000508628 | p.Trp4353* | |
| 17:80373142:C:CA | ENST00000582970, | hgvsp: p.Pro4307fs, | 1.00E−06 |
| | ENST00000508628 | p.Pro4356fs | |
| 17:80374482:C:CA | ENST00000582970, | hgvsp: p.Met4324fs, | 2.90E−06 |
| | ENST00000508628 | p.Met4373fs | |
| 17:80374494:T:TAC | ENST00000582970, | hgvsp: p.Leu4328fs, | 1.00E−06 |
| | ENST00000508628 | p.Leu4377fs | |
| 17:80374512:G:T | ENST00000582970, | hgvsp: p.Glu4333*, | 8.00E−06 |
| | ENST00000508628 | p.Glu4382* | |
| 17:80374519:A:AG | ENST00000582970, | hgvsp: p.Ala4336fs, | 1.00E−06 |
| | ENST00000508628 | p.Ala4385fs | |
| 17:80374574:TAA:T | ENST00000582970, | hgvsp: p.Lys4354fs, | 1.00E−06 |
| | ENST00000508628 | p.Lys4403fs | |
| 17:80374587:AAG:A | ENST00000582970, | hgvsp: p.Lys4358fs, | 2.70E−05 |
| | ENST00000508628 | p.Lys4407fs | |
| 17:80375764:GC:G | ENST00000582970, | hgvsp: p.Cys4360fs, | 1.60E−05 |
| | ENST00000508628 | p.Cys4409fs | |
| 17:80375813:T:TA | ENST00000582970, | hgvsp: p.Arg4377fs, | 1.00E−06 |
| | ENST00000508628 | p.Arg4426fs | |
| 17:80375827:TTTTG:T | ENST00000582970, | hgvsp: p.Leu4382fs, | 1.00E−06 |
| | ENST00000508628 | p.Leu4431fs | |
| 17:80375854:T:TC | ENST00000582970, | hgvsp: p.His4391fs, | 1.88E−06 |
| | ENST00000508628 | p.His4440fs | |
| 17:80376435:TG:T | ENST00000582970, | hgvsp: p.Gly4441fs, | 1.00E−06 |
| | ENST00000508628 | p.Gly4490fs | |
| 17:80376544:G:T | ENST00000582970, | hgvsc: c.13428 + 1G > T, | 1.00E−06 |
| | ENST00000508628 | c.13575 + 1G > T | |
| 17:80376934:G:A | ENST00000582970, | hgvsp: p.Trp4494*, | 1.00E−06 |
| | ENST00000508628 | p.Trp4543* | |
| 17:80376959:G:A | ENST00000582970, | hgvsp: p.Trp4502*, | 1.00E−06 |
| | ENST00000508628 | p.Trp4551* | |
| 17:80377782:TG:T | ENST00000582970, | hgvsp: p.Cys4511fs, | 1.00E−06 |
| | ENST00000508628 | p.Cys4560fs | |
| 17:80377790:G:GC | ENST00000582970, | hgvsp: p.Gly4514fs, | 2.91E−06 |
| | ENST00000508628 | p.Gly4563fs | |
| 17:80379638:C:T | ENST00000582970, | hgvsp: p.Gln4522*, | 1.00E−06 |
| | ENST00000508628 | p.Gln4571* | |
| 17:80379708:TG:T | ENST00000582970, | hgvsp: p.Val4546fs, | 1.00E−06 |
| | ENST00000508628 | p.Val4595fs | |
| 17:80380830:G:A | ENST00000582970, | hgvsc: c.13641 − 1G > A, | 1.00E−06 |
| | ENST00000508628 | c.13788 − 1G > A | |
| 17:80380896:CAT:C | ENST00000582970, | hgvsp: p.Cys4570fs, | 1.00E−06 |
| | ENST00000508628 | p.Cys4619fs | |
| 17:80380914:C:CCCCAGTG | ENST00000582970, | hgvsp: p.Val4578fs, | 2.00E−06 |
| | ENST00000508628 | p.Val4627fs | |
| 17:80380917:C:CA | ENST00000582970, | hgvsp: p.Val4577fs, | 1.00E−06 |
| | ENST00000508628 | p.Val4626fs | |
| 17:80380982:TC:T | ENST00000582970, | hgvsp: p.Gln4599fs, | 1.00E−06 |
| | ENST00000508628 | p.Gln4648fs | |
| 17:80380988:G:A | ENST00000582970, | hgvsc: c.13797 + 1G > A, | 1.00E−06 |
| | ENST00000508628 | c.13944 + 1G > A | |
| 17:80381545:A:T | ENST00000582970, | hgvsc: c.13798 − 2A > T, | 1.00E−06 |
| | ENST00000508628 | c.13945 − 2A > T | |
| 17:80381545:A:C | ENST00000582970, | hgvsc: c.13798 − 2A > C, | 1.00E−06 |
| | ENST00000508628 | c.13945 − 2A > C | |
| 17:80381546:G:T | ENST00000582970, | hgvsc: c.13798 − 1G > T, | 1.00E−06 |
| | ENST00000508628 | c.13945 − 1G > T | |
| 17:80381601:C:CAGCACATCCT | ENST00000582970, | hgvsp: p.Lys4622fs, | 5.00E−06 |
| | ENST00000508628 | p.Lys4671fs | |
| 17:80381614:AG:A | ENST00000582970, | hgvsp: p.Asp4623fs, | 8.51E−06 |
| | ENST00000508628 | p.Asp4672fs | |
| 17:80381706:C:T | ENST00000582970, | hgvsp: p.Gln4653*, | 1.00E−06 |
| | ENST00000508628 | p.Gln4702* | |
| 17:80382978:G:A | ENST00000582970, | hgvsc: c.13979 − 1G > A, | 1.00E−06 |
| | ENST00000508628 | c.14126 − 1G > A | |
| 17:80382997:CAG:C | ENST00000582970, | hgvsp: p.Glu4667fs, | 1.00E−06 |
| | ENST00000508628 | p.Glu4716fs | |
| 17:80383014:G:T | ENST00000582970, | hgvsp: p.Glu4672*, | 1.00E−06 |
| | ENST00000508628 | p.Glu4721* | |
| 17:80383020:AG:A | ENST00000582970, | hgvsp: p.Asn4675fs, | 1.00E−06 |
| | ENST00000508628 | p.Asn4724fs | |
| 17:80383032:GA:G | ENST00000582970, | hgvsp: p.Lys4679fs, | 3.00E−06 |
| | ENST00000508628 | p.Lys4728fs | |
| 17:80383071:G:A | ENST00000582970, | hgvsc: c.14070 + 1G > A, | 1.00E−06 |
| | ENST00000508628 | c.14217 + 1G > A | |
| 17:80383719:CAAGAT:C | ENST00000582970, | hgvsp: p.Asp4706fs, | 1.00E−06 |
| | ENST00000508628 | p.Asp4755fs | |
| 17:80383788:C:CA | ENST00000582970, | hgvsp: p.His4728fs, | 1.00E−06 |
| | ENST00000508628 | p.His4777fs | |
| 17:80383853:TG:T | ENST00000582970, | hgvsp: p.Glu4750fs, | 1.00E−06 |
| | ENST00000508628 | p.Glu4799fs | |
| 17:80383861:TC:T | ENST00000582970, | hgvsp: p.Gln4753fs, | 1.00E−06 |
| | ENST00000508628 | p.Gln4802fs | |
| 17:80383878:C:T | ENST00000582970, | hgvsp: p.Gln4758*, | 1.00E−06 |
| | ENST00000508628 | p.Gln4807* | |
| 17:80383880:G:GA | ENST00000582970, | hgvsp: p.Asn4760fs, | 8.00E−06 |
| | ENST00000508628 | p.Asn4809fs | |
| 17:80383889:CAAAG:C | ENST00000582970, | hgvsp: p.Arg4764fs, | 2.00E−06 |
| | ENST00000508628 | p.Arg4813fs | |
| 17:80383929:G:T | ENST00000582970, | hgvsc: c.14322 + 1G > T, | 1.00E−06 |
| | ENST00000508628 | c.14469 + 1G > T | |
| 17:80383930:T:C | ENST00000582970, | hgvsc: c.14322 + 2T > C, | 4.00E−06 |
| | ENST00000508628 | c.14469 + 2T > C | |
| 17:80385087:C:T | ENST00000582970, | hgvsp: p.Gln4791*, | 1.00E−06 |
| | ENST00000508628 | p.Gln4840* | |
| 17:80385143:C:CA | ENST00000582970, | hgvsp: p.Arg4810fs, | 1.00E−06 |
| | ENST00000508628 | p.Arg4859fs | |
| 17:80385144:A:T | ENST00000582970, | hgvsp: p.Arg4810*, | 1.00E−06 |
| | ENST00000508628 | p.Arg4859* | |
| 17:80385568:TCA:T | ENST00000582970, | hgvsp: p.Thr4830fs, | 1.00E−06 |
| | ENST00000508628 | p.Thr4879fs | |
| 17:80385589:G:A | ENST00000582970, | hgvsp: p.Trp4836*, | 1.00E−06 |
| | ENST00000508628 | p.Trp4885* | |
| 17:80386248:A:C | ENST00000582970, | hgvsc: c.14540 − 2A > C, | 1.00E−06 |
| | ENST00000508628 | c. 14687 − 2A > C | |
| 17:80386256:TC:T | ENST00000582970, | hgvsp: p.Asn4850fs, | 4.00E−06 |
| | ENST00000508628 | p.Asn4899fs | |
| 17:80386301:CTG:C | ENST00000582970, | hgvsp: p.Glu4865fs, | 1.00E−06 |
| | ENST00000508628 | p.Glu4914fs | |
| 17:80386301:C:CT | ENST00000582970, | hgvsp: p.Glu4865fs, | 1.00E−06 |
| | ENST00000508628 | p.Glu4914fs | |
| 17:80386301:C:CTGAGTTTGAGATCCTCT | ENST00000582970, | hgvsp: p.Pro4871fs, | 1.00E−06 |
| | ENST00000508628 | p.Pro4920fs | |
| 17:80386304:AG:A | ENST00000582970, | hgvsp: p.Glu4865fs, | 1.00E−06 |
| | ENST00000508628 | p.Glu4914fs | |
| 17:80386309:G:T | ENST00000582970, | hgvsp: p.Glu4867*, | 1.00E−06 |
| | ENST00000508628 | p.Glu4916* | |
| 17:80386400:C:CCG | ENST00000582970, | hgvsp: p.Val4898fs, | 1.00E−06 |
| | ENST00000508628 | p.Val4947fs | |
| 17:80386431:G:A | ENST00000582970, | hgvsc: c.14720 + 1G > A, | 1.00E−06 |
| | ENST00000508628 | c.14867 + 1G > A | |
| 17:80386720:AC:A | ENST00000582970, | hgvsp: p.Leu4918fs, | 5.00E−06 |
| | ENST00000508628 | p.Leu4967fs | |
| 17:80386738:T:A | ENST00000582970, | hgvsp: p.Tyr4923*, | 1.00E−06 |
| | ENST00000508628 | p.Tyr4972* | |
| 17:80386766:A:AT | ENST00000582970, | hgvsp: p.Leu4934fs, | 1.00E−06 |
| | ENST00000508628 | p.Leu4983fs | |
| 17:80386786:C:G | ENST00000582970, | hgvsp: p.Tyr4939*, | 1.00E−06 |
| | ENST00000508628 | p.Tyr4988* | |
| 17:80386801:C:CAGAG | ENST00000582970, | hgvsp: p.Thr4947fs, | 1.00E−06 |
| | ENST00000508628 | p.Thr4996fs | |
| 17:80386838:C:T | ENST00000582970, | hgvsp: p.Gln4957*, | 1.00E−06 |
| | ENST00000508628 | p.Gln5006* | |
| 17:80386884:GC:G | ENST00000582970, | hgvsp: p.Leu4973fs, | 3.00E−06 |
| | ENST00000508628 | p.Leu5022fs | |
| 17:80388611:G:A | ENST00000582970, | hgvsc: c.14923 − 1G > A, | 1.00E−06 |
| | ENST00000508628 | c.15070 − 1G > A | |
| 17:80388678:A:T | ENST00000582970, | hgvsp: p.Lys4997*, | 1.00E−06 |
| | ENST00000508628 | p.Lys5046* | |
| 17:80388690:G:C | ENST00000582970, | hgvsc: c.15000 + 1G > C, | 2.00E−06 |
| | ENST00000508628 | c.15147 + 1G > C | |
| 17:80388690:G:A | ENST00000582970, | hgvsc: c.15000 + 1G > A, | 1.00E−06 |
| | ENST00000508628 | c.15147 + 1G > A | |
| 17:80389193:CA:C | ENST00000582970, | hgvsp: p.Ile5008fs, | 1.00E−06 |
| | ENST00000508628 | p.Ile5057fs | |
| 17:80389218:C:T | ENST00000582970, | hgvsp: p.Gln5016*, | 2.00E−06 |
| | ENST00000508628 | p.Gln5065* | |
| 17:80389339:T:TG | ENST00000582970, | hgvsp: p.Asp5058fs, | 1.00E−06 |
| | ENST00000508628 | p.Asp5107fs | |
| 17:80389897:C:T | ENST00000582970, | hgvsp: p.Gln5089*, | 8.00E−06 |
| | ENST00000508628 | p.Gln5138* | |
| 17:80390159:CA:C | ENST00000582970, | hgvsp: p.Thr5146fs, | 2.00E−06 |
| | ENST00000508628 | p.Thr5195fs | |
| 17:80390159:C:T | ENST00000582970, | hgvsp: p.Gln5145*, | 3.00E−06 |
| | ENST00000508628 | p.Gln5194* | |
| 17:80390174:G:T | ENST00000582970, | hgvsp: p.Glu5150*, | 3.00E−06 |
| | ENST00000508628 | p.Glu5199* | |
| 17:80390193:G:A | ENST00000582970, | hgvsp: p.Trp5156*, | 3.00E−06 |
| | ENST00000508628 | p.Trp5205* | |
| 17:80390197:G:A | ENST00000582970, | hgvsc: c.15470 + 1G > A, | 1.00E−06 |
| | ENST00000508628 | c.15617 + 1G > A | |
| 17:80390197:G:C | ENST00000582970, | hgvsc: c.15470 + 1G > C, | 1.00E−06 |
| | ENST00000508628 | c.15617 + 1G > C | |
| 17:80393347:TGA:T | ENST00000582970, | hgvsp: p.Asp5160fs, | 1.00E−06 |
| | ENST00000508628 | p.Asp5209fs | |
| 17:80393378:TAAAG:T | ENST00000582970, | hgvsp: p.Glu5170fs, | 1.00E−06 |
| | ENST00000508628 | p.Glu5219fs | |
| 17:80393428:TA:T | ENST00000582970, | hgvsp: p.Leu5186fs, | 1.00E−06 |
| | ENST00000508628 | p.Leu5235fs | |
| 17:80393487:G:T | ENST00000582970, | hgvsp: p.Glu5205*, | 1.00E−06 |
| | ENST00000508628 | p.Glu5254* | |
| 17:80393496:T:C | ENST00000582970, | hgvsp: p.Ter5208Glnext*?, | 7.00E−06 |
| | ENST00000508628 | p.Ter5257Glnext*? | |
| 17:80263684:G:A | ENST00000582970, | hgvsp: p.Met1?, | 1.60E−05 |
| | ENST00000319921, | p.Met1?, | |
| | ENST00000508628 | p.Met1? | |
| 17:80287998:C:T | ENST00000582970, | hgvsp: p.Gln149*, | 1.60E−05 |
| | ENST00000319921, | p.Gln149*, | |
| | ENST00000508628 | p.Gln198* | |
| 17:80288205:C:T | ENST00000582970, | hgvsp: p.Gln218*, | 1.60E−05 |
| | ENST00000319921, | p.Gln218*, | |
| | ENST00000508628 | p.Gln267* | |
| 17:80294958:AG:A | ENST00000582970, | hgvsp: p.Gly571fs, | 1.60E−05 |
| | ENST00000319921, | p.Gly571fs, | |
| | ENST00000508628 | p.Gly620fs | |
| 17:80294974:TGG:T | ENST00000582970, | hgvsp: p.Trp576fs, | 1.60E−05 |
| | ENST00000319921, | p.Trp576fs, | |
| | ENST00000508628 | p.Trp625fs | |
| 17:80295725:GA:G | ENST00000582970, | hgvsp: p.Asp642fs, | 1.60E−05 |
| | ENST00000319921, | p.Asp642fs, | |
| | ENST00000508628 | p.Asp691fs | |
| 17:80306337:CCT:C | ENST00000582970, | hgvsp: p.Leu767fs, | 1.31E−05 |
| | ENST00000319921, | p.Leu767fs, | |
| | ENST00000508628 | p.Leu816fs | |
| 17:80306470:T:A | ENST00000582970, | hgvsc: c.2427 + 2T > A, | 1.60E−05 |
| | ENST00000319921, | c.2427 + 2T > A, | |
| | ENST00000508628 | c.2574 + 2T > A | |
| 17:80309133:G:T | ENST00000582970, | hgvsp: p.Glu873*, | 1.60E−05 |
| | ENST00000319921, | p.Glu873*, | |
| | ENST00000508628 | p.Glu922* | |
| 17:80313037:CTG:C | ENST00000582970, | hgvsp: p.Gly895fs, | 1.60E−05 |
| | ENST00000319921, | p.Gly895fs, | |
| | ENST00000508628 | p.Gly944fs | |
| 17:80319422:C:CA | ENST00000319921 | hgvsp: p.Glu1046fs | 1.60E−05 |
| 17:80328329:GGA:G | ENST00000582970, | hgvsp: p.Glu1124fs, | 1.60E−05 |
| | ENST00000508628 | p.Glu1173fs | |
| 17:80339592:T:TA | ENST00000582970, | hgvsp: p.Arg1743fs, | 1.60E−05 |
| | ENST00000508628 | p.Arg1792fs | |
| 17:80339717:C:T | ENST00000582970, | hgvsp: p.Gln1784*, | 1.60E−05 |
| | ENST00000508628 | p.Gln1833* | |
| 17:80339912:A:AT | ENST00000582970, | hgvsp: p.Met1849fs, | 1.31E−05 |
| | ENST00000508628 | p.Met1898fs | |
| 17:80343176:C:T | ENST00000582970, | hgvsp: p.Gln2012*, | 1.60E−05 |
| | ENST00000508628 | p.Gln2061* | |
| 17:80343222:A:AC | ENST00000582970, | hgvsp: p.Gln2029fs, | 1.60E−05 |
| | ENST00000508628 | p.Gln2078fs | |
| 17:80345599:A:ACT | ENST00000582970, | hgvsp: p.Gly2423fs, | 1.60E−05 |
| | ENST00000508628 | p.Gly2472fs | |
| 17:80345622:TATTAA:T | ENST00000582970, | hgvsp: p.Lys2431fs, | 1.60E−05 |
| | ENST00000508628 | p.Lys2480fs | |
| 17:80346327:CTT:C | ENST00000582970, | hgvsp: p.Phe2665fs, | 1.60E−05 |
| | ENST00000508628 | p.Phe2714fs | |
| 17:80346963:CA:C | ENST00000582970, | hgvsp: p.Lys2878fs, | 1.60E−05 |
| | ENST00000508628 | p.Lys2927fs | |
| 17:80353580:G:T | ENST00000582970, | hgvsp: p.Glu3498*, | 1.60E−05 |
| | ENST00000508628 | p.Glu3547* | |
| 17:80363688:TAC:T | ENST00000582970, | hgvsp: p.Gln3884fs, | 1.31E−05 |
| | ENST00000508628 | p.Gln3933fs | |
| 17:80369769:AC:A | ENST00000582970, | hgvsp: p.His4110fs, | 1.60E−05 |
| | ENST00000508628 | p.His4159fs | |
| 17:80374495:A:AC | ENST00000582970, | hgvsp: p.Leu4328fs, | 1.60E−05 |
| | ENST00000508628 | p.Leu4377fs | |
| 17:80376954:C:CA | ENST00000582970, | hgvsp: p.His4501fs, | 1.31E−05 |
| | ENST00000508628 | p.His4550fs | |
| 17:80380891:G:GGTGACAT | ENST00000582970, | hgvsp: p.Arg4572fs, | 1.60E−05 |
| | ENST00000508628 | p.Arg4621fs | |
| 17:80381644:GAC:G | ENST00000582970, | hgvsp: p.His4633fs, | 1.60E−05 |
| | ENST00000508628 | p.His4682fs | |
| 17:80386345:T:TGA | ENST00000582970, | hgvsp: p.Cys4879fs, | 3.20E−05 |
| | ENST00000508628 | p.Cys4928fs | |
| 17:80273288:ATGGAGTG:A | ENST00000582970, | hgvsp: p.Glu50fs, | 1.10E−05 |
| | ENST00000319921, | p.Glu50fs, | |
| | ENST00000508628 | p.Glu50fs | |
| 17:80278763:G:A | ENST00000508628 | hgvsc: c.262 − 1G > A | 1.10E−05 |
| 17:80288756:G:C | ENST00000582970, | hgvsc: c.933 + 1G > C, | 3.40E−05 |
| | ENST00000319921, | c.933 + 1G > C, | |
| | ENST00000508628 | c.1080 + 1G > C | |
| 17:80290656:TC:T | ENST00000582970, | hgvsp: p.Phe401fs, | 1.10E−05 |
| | ENST00000319921, | p.Phe401fs, | |
| | ENST00000508628 | p.Phe450fs | |
| 17:80295630:T:A | ENST00000582970, | hgvsp: p.Leu610*, | 1.10E−05 |
| | ENST00000319921, | p.Leu610*, | |
| | ENST00000508628 | p.Leu659* | |
| 17:80298443:G:A | ENST00000582970, | hgvsp: p.Trp712*, | 1.10E−05 |
| | ENST00000319921, | p.Trp712*, | |
| | ENST00000508628 | p.Trp761* | |
| 17:80306351:G:GGT | ENST00000582970, | hgvsp: p.Met772fs, | 1.10E−05 |
| | ENST00000319921, | p.Met772fs, | |
| | ENST00000508628 | p.Met821fs | |
| 17:80306454:GT:G | ENST00000582970, | hgvsp: p.Val805fs, | 1.10E−05 |
| | ENST00000319921, | p.Val805fs, | |
| | ENST00000508628 | p.Val854fs | |
| 17:80313058:CA:C | ENST00000582970, | hgvsp: p.Val902fs, | 1.10E−05 |
| | ENST00000319921, | p.Val902fs, | |
| | ENST00000508628 | p.Val951fs | |
| 17:80313094:G:A | ENST00000582970, | hgvsp: p.Trp913*, | 3.40E−05 |
| | ENST00000319921, | p.Trp913*, | |
| | ENST00000508628 | p.Trp962* | |
| 17:80319333:G:A | ENST00000319921 | hgvsp: p.Trp1015* | 1.10E−05 |
| 17:80328330:GA:G | ENST00000582970, | hgvsp: p.Ser1126fs, | 1.10E−05 |
| | ENST00000508628 | p.Ser1175fs | |
| 17:80332452:A:T | ENST00000582970, | hgvsp: p.Lys1322*, | 1.10E−05 |
| | ENST00000508628 | p.Lys1371* | |
| 17:80332618:CTT:C | ENST00000582970, | hgvsp: p.Leu1378fs, | 1.10E−05 |
| | ENST00000508628 | p.Leu1427fs | |
| 17:80334156:G:T | ENST00000582970, | hgvsp: p.Glu1399*, | 1.10E−05 |
| | ENST00000508628 | p.Glu1448* | |
| 17:80339293:C:G | ENST00000582970, | hgvsp: p.Tyr1642*, | 1.10E−05 |
| | ENST00000508628 | p.Tyr1691* | |
| 17:80344930:C:T | ENST00000582970, | hgvsp: p.Gln2199*, | 1.10E−05 |
| | ENST00000508628 | p.Gln2248* | |
| 17:80344934:AT:A | ENST00000582970, | hgvsp: p.Phe2201fs, | 1.10E−05 |
| | ENST00000508628 | p.Phe2250fs | |
| 17:80345138:AC:A | ENST00000582970, | hgvsp: p.His2268fs, | 1.10E−05 |
| | ENST00000508628 | p.His2317fs | |
| 17:80346302:C:CG | ENST00000582970, | hgvsp: p.Met2657fs, | 1.10E−05 |
| | ENST00000508628 | p.Met2706fs | |
| 17:80346443:AT:A | ENST00000582970, | hgvsp: p.Arg2704fs, | 1.10E−05 |
| | ENST00000508628 | p.Arg2753fs | |
| 17:80347067:TAGAG:T | ENST00000582970, | hgvsp: p.Glu2912fs, | 1.10E−05 |
| | ENST00000508628 | p.Glu2961fs | |
| 17:80348287:G:A | ENST00000582970, | hgvsc: c.9951 + 1G > A, | 1.10E−05 |
| | ENST00000508628 | c.10098 + 1G > A | |
| 17:80349812:AT:A | ENST00000582970, | hgvsp: p.Leu3333fs, | 1.10E−05 |
| | ENST00000508628 | p.Leu3382fs | |
| 17:80351788:GT:G | ENST00000582970, | hgvsp: p.Val3430fs, | 1.10E−05 |
| | ENST00000508628 | p.Val3479fs | |
| 17:80353510:A:T | ENST00000582970, | hgvsc: c.10424 − 2A > T, | 1.10E−05 |
| | ENST00000508628 | c.10571 − 2A > T | |
| 17:80354503:C:T | ENST00000582970, | hgvsp: p.Gln3597*, | 1.10E−05 |
| | ENST00000508628 | p.Gln3646* | |
| 17:80354522:G:A | ENST00000582970, | hgvsp: p.Trp3603*, | 1.10E−05 |
| | ENST00000508628 | p.Trp3652* | |
| 17:80358405:G:A | ENST00000582970, | hgvsp: p.Trp3660*, | 1.10E−05 |
| | ENST00000508628 | p.Trp3709* | |
| 17:80363694:T:A | ENST00000582970, | hgvsp: p.Leu3885*, | 2.30E−05 |
| | ENST00000508628 | p.Leu3934* | |
| 17:80364501:G:GCGTC | ENST00000582970, | hgvsp: p.Pro3942fs, | 1.10E−05 |
| | ENST00000508628 | p.Pro3991fs | |
| 17:80364515:C:T | ENST00000582970, | hgvsp: p.Gln3945*, | 2.30E−05 |
| | ENST00000508628 | p.Gln3994* | |
| 17:80367754:CGA:C | ENST00000582970, | hgvsp: p.Asn3962fs, | 1.10E−05 |
| | ENST00000508628 | p.Asn4011fs | |
| 17:80371933:TCA:T | ENST00000582970, | hgvsp: p.Ile4163fs, | 1.10E−05 |
| | ENST00000508628 | p.Ile4212fs | |
| 17:80375795:C:G | ENST00000582970, | hgvsp: p.Tyr4370*, | 1.10E−05 |
| | ENST00000508628 | p.Tyr4419* | |
| 17:80382993:GAC:G | ENST00000582970, | hgvsp: p.Thr4666fs, | 1.10E−05 |
| | ENST00000508628 | p.Thr4715fs | |
| 17:80383072:T:C | ENST00000582970, | hgvsc: c.14070 + 2T > C, | 1.10E−05 |
| | ENST00000508628 | c.14217 + 2T > C | |
| 17:80385544:TG:T | ENST00000582970, | hgvsp: p.Arg4822fs, | 1.10E−05 |
| | ENST00000508628 | p.Arg4871fs | |
| 17:80393456:G:A | ENST00000582970, | hgvsp: p.Trp5194*, | 1.10E−05 |
| | ENST00000508628 | p.Trp5243* | |
| 17:80393480:G:A | ENST00000582970, | hgvsp: p.Trp5202*, | 1.10E−05 |
| | ENST00000508628 | p.Trp5251* | |
| 17:80273317:AG:A | ENST00000582970, | hgvsp: p.Gly60fs, | 1.70E−05 |
| | ENST00000319921, | p.Gly60fs, | |
| | ENST00000508628 | p.Gly60fs | |
| 17:80290689:C:G | ENST00000582970, | hgvsp: p.Ser411*, | 1.70E−05 |
| | ENST00000319921, | p.Ser411*, | |
| | ENST00000508628 | p.Ser460* | |
| 17:80313053:CCAAA:C | ENST00000582970, | hgvsp: p.Thr901fs, | 1.70E−05 |
| | ENST00000319921, | p.Thr901fs, | |
| | ENST00000508628 | p.Thr950fs | |
| 17:80327814:A:C | ENST00000582970, | hgvsc: c.3194 − 2A > C, | 0.00017 |
| | ENST00000508628 | c.3341 − 2A > C | |
| 17:80328326:A:AG | ENST00000582970, | hgvsc: c.3368 − 2_3368 − 1insG, | 1.70E−05 |
| | ENST00000508628 | c.3515 − 2_3515 − 1insG | |
| 17:80339214:T:TG | ENST00000582970, | hgvsp: p.Gln1617fs, | 0.000119 |
| | ENST00000508628 | p.Gln1666fs | |
| 17:80339640:GGA:G | ENST00000582970, | hgvsp: p.Arg1759fs, | 1.70E−05 |
| | ENST00000508628 | p.Arg1808fs | |
| 17:80343136:GTC:G | ENST00000582970, | hgvsp: p.Leu2000fs, | 0.000153 |
| | ENST00000508628 | p.Leu2049fs | |
| 17:80343304:TC:T | ENST00000582970, | hgvsp: p.His2055fs, | 1.70E−05 |
| | ENST00000508628 | p.His2104fs | |
| 17:80345571:T:A | ENST00000582970, | hgvsp: p.Cys2412*, | 5.10E−05 |
| | ENST00000508628 | p.Cys2461* | |
| 17:80345692:G:T | ENST00000582970, | hgvsp: p.Gly2453*, | 3.40E−05 |
| | ENST00000508628 | p.Gly2502* | |
| 17:80347528:C:T | ENST00000582970, | hgvsp: p.Gln3065*, | 5.10E−05 |
| | ENST00000508628 | p.Gln3114* | |
| 17:80349769:G:A | ENST00000582970, | hgvsc: c.9952 − 1G > A, | 1.70E−05 |
| | ENST00000508628 | c.10099 − 1G > A | |
| 17:80354564:CG:C | ENST00000582970, | hgvsp: p.Gly3618fs, | 5.10E−05 |
| | ENST00000508628 | p.Gly3667fs | |
| 17:80371948:AAACTG:A | ENST00000582970, | hgvsp: p.Glu4169fs, | 3.40E−05 |
| | ENST00000508628 | p.Glu4218fs | |
| 17:80372620:CG:C | ENST00000582970, | hgvsp: p.Gly4214fs, | 1.70E−05 |
| | ENST00000508628 | p.Gly4263fs | |
| 17:80372714:C:A | ENST00000582970, | hgvsp: p.Ser4244*, | 1.70E−05 |
| | ENST00000508628 | p.Ser4293* | |
| 17:80373015:GC:G | ENST00000582970, | hgvsp: p.Gln4265fs, | 1.70E−05 |
| | ENST00000508628 | p.Gln4314fs | |
| 17:80374457:G:A | ENST00000582970, | hgvsc: c.12943 − 1G > A, | 1.70E−05 |
| | ENST00000508628 | c.13090 − 1G > A | |
| 17:80374590:G:A | ENST00000582970, | hgvsc: c.13074 + 1G > A, | 1.70E−05 |
| | ENST00000508628 | c.13221 + 1G > A | |
| 17:80375871:G:C | ENST00000582970, | hgvsc: c.13185 + 1G > C, | 1.70E−05 |
| | ENST00000508628 | c.13332 + 1G > C | |
| 17:80376440:CG:C | ENST00000582970, | hgvsp: p.Val4443fs, | 1.70E−05 |
| | ENST00000508628 | p.Val4492fs | |
| 17:80376478:CTG:C | ENST00000582970, | hgvsp: p.Cys4456fs, | 3.40E−05 |
| | ENST00000508628 | p.Cys4505fs | |
| 17:80386300:ACT:A | ENST00000582970, | hgvsp: p.Thr4864fs, | 1.70E−05 |
| | ENST00000508628 | p.Thr4913fs | |
| 17:80386801:CAG:C | ENST00000582970, | hgvsp: p.Glu4946fs, | 1.70E−05 |
| | ENST00000508628 | p.Glu4995fs | |
| 17:80386848:TC:T | ENST00000582970, | hgvsp: p.Ile4960fs, | 1.70E−05 |
| | ENST00000508628 | p.Ile5009fs | |
| 17:80390041:C:G | ENST00000582970, | hgvsp: p.Tyr5105*, | 1.70E−05 |
| | ENST00000508628 | p.Tyr5154* | |
| 17:80393343:A:G | ENST00000582970, | hgvsc: c.15471 − 2A > G, | 1.70E−05 |
| | ENST00000508628 | c.15618 − 2A > G | |

Protein changes follow the recommendation of the Human Genome Variation Society and correspond to each to the Ensembl transcript IDs, hgvsp (protein change) is given in case of a protein coding variant, hgvsc (cDNA change) is given in case of a splice variant. AAF indicates the alternative allele frequency.

Table 5 shows that loss of function alleles in RNF213 are strongly associated with protection against liver diseases diagnoses, including parenchymal liver disease, alcoholic, non-alcoholic liver disease, liver fibrosis and cirrhosis of the liver. These results indicate that loss-of-function of RNF213 protects against chronic liver diseases.

TABLE 5
Associations between RNF213 pLOF and the most significant ALT associated
common coding variant Val3838Leu and various clinical diagnoses of liver
disease in a meta- analysis of the UKB, GHS, SINAI, MDCS and UPENN-PMBB

| | | Per allele | | | |
|---|---|---|---|---|---|
| | | Effect OR (95% | | Genotype counts, | |
| Genetic | | confidence | | RR\|RA\|AA | |
| exposure | Outcome | interval) | P-value | genotypes | AAF |
| pLOF | Alcoholic | 0.88 | 7.05E−01 | cases: | 0.00206 |
| | liver disease | (0.46, 1.70) | | 2,155\|8\|0 | |
| | | | | controls: | |
| | | | | 415,506\|1,521\|1 | |
| pLOF | Any liver | 0.78 | 1.30E−02 | cases: | 0.00242 |
| | disease | (0.65, 0.95) | | 26,764\|102\|0 | |
| | | | | controls: | |
| | | | | 466,358\|1,850\|2 | |
| pLOF | Liver cirrhosis | 0.65 | 4.30E−02 | cases: | 0.00277 |
| | | (0.43, 0.99) | | 4,554\|15\|0 | |
| | | | | controls: | |
| | | | | 444,787\|1,766\|2 | |
| pLOF | Alcoholic | 0.79 | 6.01E−01 | cases: | 0.00215 |
| | liver cirrhosis | (0.33, 1.90) | | 1,162\|4\|0 | |
| | | | | controls: | |
| | | | | 387,820\|1,383\|1 | |
| pLOF | Non alcoholic | 0.64 | 4.76E−02 | cases: | 0.00286 |
| | liver cirrhosis | (0.41, 1.00) | | 3,970\|13\|0 | |
| | | | | controls: | |
| | | | | 444,552\|1,764\|2 | |
| pLOF | Non-alcoholic | 0.72 | 4.53E−02 | cases: | 0.00243 |
| | fatty liver | (0.53, 0.99) | | 7,887\|28\|0 | |
| | disease and | | | controls: | |
| | steatosis | | | 413,423\|1,608\|2 | |
| | hepatitis | | | | |
| pLOF | Non alcoholic | 0.73 | 1.08E−02 | cases: | 0.00257 |
| | liver disease | (0.57, 0.93) | | 14,845\|54\|0 | |
| | | | | controls: | |
| | | | | 444,787\|1,766\|2 | |
| pLOF | Parenchymal | 0.77 | 2.94E−02 | cases: | 0.00248 |
| | liver disease | (0.61, 0.97) | | 16,984\|64\|0 | |
| | | | | controls: | |
| | | | | 438,672\|1,712\|2 | |
| p.Val3838Leu | Alcoholic | 0.96 | 4.43E−01 | cases: | 0.09731 |
| | liver disease | (0.87, 1.06) | | 1,791\|348\|24 | |
| | | | | controls: | |
| | | | | 341,045\|72,093\|3,890 | |
| p.Val3838Leu | Any liver | 0.97 | 4.64E−02 | cases: | 0.11008 |
| | disease | (0.94, 1.00) | | 21,406\|5,144\|320 | |
| | | | | controls: | |
| | | | | 378,557\|84,421\|5,239 | |
| p.Val3838Leu | Liver cirrhosis | 0.87 | 3.40E−05 | cases: | 0.12255 |
| | | (0.81, 0.93) | | 3,6521855165 | |
| | | | | controls: | |
| | | | | 360,859\|80,678\|5,025 | |
| p.Val3838Leu | Alcoholic | 0.87 | 4.63E−02 | cases: | 0.09922 |
| | liver cirrhosis | (0.76, 1.00) | | 978\|176\|12 | |
| | | | | controls: | |
| | | | | 318,031\|67,490\|3,683 | |
| p.Val3838Leu | Non alcoholic | 0.87 | 1.40E−04 | cases: | 0.12529 |
| | liver cirrhosis | (0.81, 0.93) | | 3,166\|761\|59 | |
| | | | | controls: | |
| | | | | 360,674\|80,629\|5,022 | |
| p.Val3838Leu | Non-alcoholic | 0.97 | 1.78E−01 | cases: | 0.10807 |
| | fatty liver | (0.92, 1.02) | | 6,340\|1,491\|84 | |
| | disease and | | | controls: | |
| | steatosis | | | 334,882\|75,382\|4,776 | |
| | hepatitis | | | | |
| p.Val3838Leu | Non alcoholic | 0.94 | 1.78E−03 | cases: | 0.11211 |
| | liver disease | (0.90, 0.98) | | 11,892\|2,830\|180 | |
| | | | | controls: | |
| | | | | 360,859\|80,678\|5,025 | |
| p.Val3838Leu | Parenchymal | 0.96 | 4.69E−02 | cases: | 0.10934 |
| | liver disease | (0.93, 1.00) | | 13,620\|3,236\|195 | |
| | | | | controls: | |
| | | | | 355,543\|79,818\|5,032 | |

RR indicates the number of individuals in the population studies carrying no alternative alleles; RA indicates the number of individuals carrying one or more heterozygous alternative alleles; AA indicates the number of individuals carrying one or more homozygous alternative alleles; The alternative allele is the allele causing loss of function or change in amino acid as coded following HGVS recommendations; The alternative allele is the allele causing loss of function or change in amino acid as coded following HGVS recommendations; OR indicates odds ratio; AAF indicates the alternative allele frequency.

### Participating Cohorts

Genetic association studies were performed in the United Kingdom Biobank (UKB) cohort (Sudlow et al., PLoS Med., 2015, 12, e1001779) and the DiscoverEHR cohort from the Geisinger Health System (GHS) MyCode Community Health Initiative (Carey et al., Genet. Med., 2016, 18, 906-13). UKB is a population-based cohort study of people aged between 40 and 69 years recruited through 22 testing centers in the UK between 2006-2010. Over 430,000 European ancestry participants from UKB with available whole-exome sequencing and clinical phenotype data were included. The GHS MyCode study Community Health Initiative is a health system-based cohort of patients from Central and Eastern Pennsylvania (USA) recruited in 2007-2019. Over 130,000 European ancestry participants from GHS with available whole-exome sequencing and clinical phenotype data were included. The associations with liver outcomes also included the Mount Sinai BioMe Biobank cohort (SINAI, Cell, 2019, 177, 58-69), The University of Pennsylvania Penn Medicine BioBank (UPENN-PMBB; (Park et al., 2020, doi:10.1038/s41436-019-0625-8)) and Malmo Diet and Cancer Study (MDCS) a Swedish population-based, prospective, observational cohort recruited between 1991 and 1996 (Berglund et al., 1993, doi:10.1111/j.1365-2796.1993.tb00647.x).

### Phenotype Definitions

Clinical laboratory measurements for ALT or AST were extracted from electronic health records (EHRs) of participants from GHS. Median values were calculated for all participants with two or more measurements. In UKB, ALT and AST were measured by IFCC (International Federation of Clinical Chemistry) analysis on a Beckman Coulter AU5800 at the baseline visit of the study; Hb1Ac was measured by HPLC using a Bio-Rad VARIANT II Turbo. Prior to genetic association analysis, continues phenotype values were transformed by the inverse standard normal function, applied within each ancestry group and separately in men and women. Disease outcomes were defined according to the International Classification of Diseases, Ninth and Tenth Revision (ICD-9 and ICD-10) using EHRs and self-reports when available and combined into single variables as described in Table 6.

TABLE 6
Definitions of liver disease outcomes in
UKB, GHS, SINAI, UPENN-PMBB and MALMO

| Liver disease | | Controls |
|---|---|---|
| outcome | Case definition | definition |
| Any liver disease | ICD10: | See footnote* |
| | K70, K71, K72, K73, K74, K75, | |
| | K76, K77, I81, I85, I982, | |
| | I983, I864, T864, Z944, C220 | |
| | UKB.OPCS4: G10, G144, J01 | |
| | UKB.f.20002: 1604, 1158, 1141 | |
| Parenchymal liver | ICD10: | See footnote* |
| disease | K70, K71, K72, K73, K74, K753, | |
| | K753, K752, K754, K758, | |
| | K759, K760, K767, K7681 | |
| | OPCS4: G10, G144, J01 | |
| | UKB.f.20002: 1604, 1158, 1141 | |
| Alcoholic liver | ICD10: K70 | See footnote* |
| disease | | |
| Non-alcoholic liver | ICD10: | See footnote* |
| disease | K721, K740, K741, K742, K746, | |
| | K758, K760 | |
| Liver cirrhosis (any | ICD10: | See footnote* |
| etiology) | K703, K704, K717, K721, K746 | |
| Alcoholic liver | ICD10: K703, K704 | See footnote* |
| cirrhosis | | |
| Non-alcoholic liver | ICD10: K746 | See footnote* |
| cirrhosis | | |
| NAFLD/NASH | ICD10: K760, K7581 | See footnote* |
| Viral hepatitis | ICD10: K746, K758, K760 | See footnote* |

*Participants were excluded from the control population if they were diagnosed with the “any liver disease” outcome codes (as defined in the table) or if they had elevated ALT >33 U/L for men and >25 U/L for women.

ICD10 indicates the 10th revision of the International Statistical Classification of Diseases and Related Health Problems; UKB.OPCS4 indicates Office of Population Censuses and Surveys (OPCS) Classification of Interventions and Procedures version 4 as used in the UK Biobank (UKB); UKB.f.20002 indicates self-reported non-cancer illness codes as used in UKB. UKB.f.20004 indicates self-reported medical procedures as used in UKB.

### Genotype Data

High coverage whole exome sequencing was performed as previously described (Science, 2016, 354:aaf6814; and Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

### Association Analysis of Gene Burden of Rare Loss of Function Variation

Association between the burden of rare predicted loss-of-function or missense variants in a given gene and phenotype was examined by fitting a linear (for quantitative traits) or firth bias-corrected logistic (for binary traits) regression model adjusted for a polygenic score that approximates a genomic kinship matrix using REGENIE v1.0 (doi: “world wide web” at “doi.org/10.1101/2020.06.19.162354”). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals were labeled as heterozygotes if they carried one or more qualifying rare variant (as described above based on frequency and functional annotation) and as homozygotes if they carried any qualifying variant in the homozygous state. This “composite genotype” was then used to test for association.

### GWAS of Common Variants and Fine-Mapping Independent Signals

Associated common variants were identified by performing a genome-wide association study including over 12 million common-to-low-frequency genetic variants imputed using the Haplotype Reference Consortium panel. In the GHS study, imputation was performed separately in samples genotyped with the Illumina Human Omni Express Exome array (OMNI set) and the Global Screening array (GSA set). Dosage data from imputed variants were then merged across the two GHS sets, to obtain a combined dataset for association analysis. Genome-wide association analyses were performed in GHS and UKB separately by fitting whole genome regression models using REGENIE (Mbatchou et al., 2020, doi:10.1101/2020.06.19.162354). As described above for burden tests, within each cohort analyses were stratified by ancestry and adjusted for age, age², sex, age-by-sex and age²-by-sex interaction terms, experimental batch-related covariates, and 10 common variant-derived principal components. Results from the UKB and GHS analyses were then combined by inverse variance-weighted meta-analysis to obtain a genome-wide meta-analysis in the European subset of the discovery cohorts. To identify conditionally independent genetic association signals driven by common variants, fine-mapping was performed at genomic regions harboring genetic variants associated with each trait of interest at the genome-wide significance threshold of p<5×10⁻⁰⁸ using FINEMAP (Benner et al., 2016, doi:10.1093/bioinformatics/btw018). Linkage disequilibrium was estimated using genetic data from the exact set of individuals included in the genome-wide association analyses. Fine-mapping was performed separately in the meta-analysis of the European ancestry GHS and UKB cohorts. Fine-mapping identifies independent common variant signals and assigns a posterior probability of causal association for variants linked to a given independent signal. For each locus that was fine-mapped, the 95% credible set of causal variants (i.e., the minimal set of variants that capture the 95% posterior probability of causal association) were identified. The sentinel variant was defined as the variant with the highest posterior probability of causal association at each given independent signal.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

What is claimed is:

1. A method of treating a subject having liver disease, the method comprising administering a Ring Finger Protein 213 (RNF213) inhibitor to the subject.

2. A method of treating a subject having fatty liver disease, the method comprising administering a Ring Finger Protein 213 (RNF213) inhibitor to the subject.

3. The method according to claim 2, wherein the fatty liver disease is alcoholic fatty liver disease (AFLD) or nonalcoholic fatty liver disease (NAFLD).

4. A method of treating a subject having hepatocellular carcinoma, the method comprising administering a Ring Finger Protein 213 (RNF213) inhibitor to the subject.

5. A method of treating a subject having liver cirrhosis, the method comprising administering a Ring Finger Protein 213 (RNF213) inhibitor to the subject.

6. A method of treating a subject having liver fibrosis, the method comprising administering a Ring Finger Protein 213 (RNF213) inhibitor to the subject.

7. A method of treating a subject having simple steatosis, steatohepatitis, or non-alcoholic steatohepatitis (NASH), the method comprising administering a Ring Finger Protein 213 (RNF213) inhibitor to the subject.

8. The method according to any one of claims 1 to 7, wherein the RNF213 inhibitor comprises an antisense nucleic acid molecule, a small interfering RNA (siRNA), or a short hairpin RNA (shRNA) that hybridizes to an RNF213 mRNA.

9. The method according to any one of claims 1 to 7, wherein the RNF213 inhibitor comprises a Cas protein and guide RNA (gRNA) that hybridizes to a gRNA recognition sequence within an RNF213 genomic nucleic acid molecule.

10. The method according to claim 9, wherein the Cas protein is Cas9 or Cpf1.

11. The method according to claim 9 or claim 10, wherein the gRNA recognition sequence includes or is proximate to a position corresponding to: position 102,917 according to SEQ ID NO:1, position 102,391 according to SEQ ID NO:1, or position 103,226 according to SEQ ID NO:1.

12. The method according to claim 9 or claim 10, wherein the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 102,917 according to SEQ ID NO:1, position 102,391 according to SEQ ID NO:1, or position 103,226 according to SEQ ID NO:1.

13. The method according to claim 9 or claim 10, wherein a Protospacer Adjacent Motif (PAM) sequence is about 2 to about 6 nucleotides downstream of the gRNA recognition sequence.

14. The method according to any one of claims 9 to 13, wherein the gRNA comprises from about 17 to about 23 nucleotides.

15. The method according to any one of claims 9 to 13, wherein the gRNA recognition sequence comprises a nucleotide sequence according to any one of SEQ ID NOS:62-81.

16. The method according to any one of claims 1 to 15, further comprising detecting the presence or absence of an RNF213 predicted loss-of-function or missense variant nucleic acid molecule encoding a human RNF213 polypeptide in a biological sample from the subject.

17. The method according to claim 16, wherein when the subject is RNF213 reference, the subject is also administered a therapeutic agent that treats or inhibits a liver disease in a standard dosage amount.

18. The method according to claim 16, wherein when the subject is heterozygous for an RNF213 predicted loss-of-function or missense variant, the subject is also administered a therapeutic agent that treats or inhibits a liver disease in a dosage amount that is the same as or lower than a standard dosage amount.

19. The method according to any one of claims 16 to 18, wherein the RNF213 predicted loss-of-function or missense variant nucleic acid molecule is a nucleic acid molecule encoding Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu.

20. The method according to any one of claims 16 to 18, wherein the RNF213 predicted loss-of-function or missense variant nucleic acid molecule is a nucleic acid molecule encoding Glu3915Gly or Val3838Leu.

21. The method according to claim 19, wherein the RNF213 predicted loss-of-function or missense variant nucleic acid molecule is:
a genomic nucleic acid molecule having a nucleotide sequence comprising: a guanine at a position corresponding to position 102,917 according to SEQ ID NO:2; a cytosine at a position corresponding to position 102,391 according to SEQ ID NO:3; or a thymine at a position corresponding to position 103,226 according to SEQ ID NO:4;
an mRNA molecule having a nucleotide sequence comprising: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23; or
a cDNA molecule produced from an mRNA molecule, wherein the cDNA molecule has a nucleotide sequence comprising: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

22. The method according to any one of claims 16 to 21, wherein the detecting step is carried out in vitro.

23. The method according to any one of claims 16 to 22, wherein the detecting step comprises sequencing at least a portion of the nucleotide sequence of the RNF213 genomic nucleic acid molecule in the biological sample, wherein the sequenced portion comprises a position corresponding to: position 102,917 according to SEQ ID NO:2, or the complement thereof; position 102,391 according to SEQ ID NO:3, or the complement thereof; or position 103,226 according to SEQ ID NO:4, or the complement thereof;
wherein when the sequenced portion of the RNF213 genomic nucleic acid molecule in the biological sample comprises: a guanine at a position corresponding to position 102,917 according to SEQ ID NO:2; a cytosine at a position corresponding to position 102,391 according to SEQ ID NO:3; or a thymine at a position corresponding to position 103,226 according to SEQ ID NO:4, then the RNF213 genomic nucleic acid molecule in the biological sample is an RNF213 predicted loss-of-function or missense variant genomic nucleic acid molecule.

24. The method according to any one of claims 16 to 22, wherein the detecting step comprises sequencing at least a portion of the nucleotide sequence of the RNF213 mRNA molecule in the biological sample, wherein the sequenced portion comprises a position corresponding to: position 11,887 according to SEQ ID NO:12, or the complement thereof; position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof; or position 84 according to SEQ ID NO:18, or the complement thereof; position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof; or position 206 according to SEQ ID NO:23, or the complement thereof;
wherein when the sequenced portion of the RNF213 mRNA molecule in the biological sample comprises: a guanine at a position corresponding to position 11,887 according to SEQ ID NO:12; a guanine at a position corresponding to position 12,036 according to SEQ ID NO:13; a guanine at a position corresponding to position 2,685 according to SEQ ID NO:14; a guanine at a position corresponding to position 1,050 according to SEQ ID NO:15; a guanine at a position corresponding to position 438 according to SEQ ID NO:16; a guanine at a position corresponding to position 112 according to SEQ ID NO:17; a guanine at a position corresponding to position 84 according to SEQ ID NO:18; a cytosine at a position corresponding to position 11,655 according to SEQ ID NO:19; a cytosine at a position corresponding to position 11,804 according to SEQ ID NO:20; a cytosine at a position corresponding to position 2,453 according to SEQ ID NO:21; a cytosine at a position corresponding to position 818 according to SEQ ID NO:22; or a cytosine at a position corresponding to position 206 according to SEQ ID NO:23; then the RNF213 mRNA molecule in the biological sample is an RNF213 predicted loss-of-function or missense variant mRNA molecule.

25. The method according to any one of claims 16 to 22, wherein the detecting step comprises sequencing at least a portion of the nucleotide sequence of the RNF213 cDNA molecule produced from an mRNA molecule in the biological sample, wherein the sequenced portion comprises a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof; or position 84 according to SEQ ID NO:37, or the complement thereof; position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof;
wherein when the sequenced portion of the RNF213 cDNA molecule in the biological sample comprises: a guanine at a position corresponding to position 11,887 according to SEQ ID NO:31; a guanine at a position corresponding to position 12,036 according to SEQ ID NO:32; a guanine at a position corresponding to position 2,685 according to SEQ ID NO:33; a guanine at a position corresponding to position 1,050 according to SEQ ID NO:34; a guanine at a position corresponding to position 438 according to SEQ ID NO:35; a guanine at a position corresponding to position 112 according to SEQ ID NO:36; a guanine at a position corresponding to position 84 according to SEQ ID NO:37; a cytosine at a position corresponding to position 11,655 according to SEQ ID NO:38; a cytosine at a position corresponding to position 11,804 according to SEQ ID NO:39; a cytosine at a position corresponding to position 2,453 according to SEQ ID NO:40; a cytosine at a position corresponding to position 818 according to SEQ ID NO:41; or a cytosine at a position corresponding to position 206 according to SEQ ID NO:42; then the RNF213 cDNA molecule in the biological sample is an RNF213 predicted loss-of-function or missense variant cDNA molecule.

26. The method according to any one of claims 16 to 22, wherein the detecting step comprises:
a) contacting the biological sample with a primer hybridizing to a portion of the nucleotide sequence of the RNF213 genomic nucleic acid molecule that is proximate to a position corresponding to: position 102,917 according to SEQ ID NO:2, position 102,391 according to SEQ ID NO:3, or position 103,226 according to SEQ ID NO:4;
b) extending the primer at least through the position of the nucleotide sequence of the RNF213 genomic nucleic acid molecule corresponding to: position 102,917 according to SEQ ID NO:2, position 102,391 according to SEQ ID NO:3, or position 103,226 according to SEQ ID NO:4; and
c) determining whether the extension product of the primer comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4.

27. The method according to any one of claims 16 to 22, wherein the detecting step comprises:
a) contacting the biological sample with a primer hybridizing to a portion of the nucleotide sequence of the RNF213 mRNA molecule that is proximate to a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23;
b) extending the primer at least through the position of the nucleotide sequence of the RNF213 mRNA molecule corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and
c) determining whether the extension product of the primer comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23.

28. The method according to any one of claims 16 to 22, wherein the detecting step comprises:
a) contacting the biological sample with a primer hybridizing to a portion of the nucleotide sequence of the RNF213 cDNA molecule that is proximate to a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42;
b) extending the primer at least through the position of the nucleotide sequence of the RNF213 cDNA molecule corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; and
c) determining whether the extension product of the primer comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

29. The method according to any one of claims 23 to 28, wherein the detecting step comprises sequencing the entire nucleic acid molecule.

30. The method according to any one of claims 16 to 22, wherein the detecting step comprises:
a) amplifying at least a portion of the genomic nucleic acid molecule that encodes the human RNF213 polypeptide, wherein the portion comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
d) detecting the detectable label.

31. The method according to any one of claims 16 to 22, wherein the detecting step comprises:
a) amplifying at least a portion of the mRNA molecule that encodes the human RNF213 polypeptide, wherein the portion comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and
d) detecting the detectable label.

32. The method according to any one of claims 16 to 22, wherein the detecting step comprises:
a) amplifying at least a portion of the cDNA molecule that encodes the human RNF213 polypeptide, wherein the portion comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
d) detecting the detectable label.

33. The method according to claim 32, wherein the nucleic acid molecule in the sample is mRNA and the mRNA is reverse-transcribed into cDNA prior to the amplifying step.

34. The method according to any one of claims 16 to 22, wherein the detecting step comprises:
contacting the genomic nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
detecting the detectable label.

35. The method according to any one of claims 16 to 22, wherein the detecting step comprises:
contacting the mRNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and
detecting the detectable label.

36. The method according to any one of claims 16 to 22, wherein the detecting step comprises:
contacting the cDNA molecule produced from an mRNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
detecting the detectable label.

37. A method of treating a subject with a therapeutic agent that treats or inhibits a liver disease, wherein the subject is suffering from a liver disease, the method comprising the steps of:
determining whether the subject has a Ring Finger Protein 213 (RNF213) predicted loss-of-function or missense variant nucleic acid molecule encoding a human RNF213 polypeptide by:
obtaining or having obtained a biological sample from the subject; and
performing or having performed a genotyping assay on the biological sample to determine if the subject has a genotype comprising the RNF213 predicted loss-of-function or missense variant nucleic acid molecule; and
when the subject is RNF213 reference, then administering or continuing to administer to the subject the therapeutic agent that treats or inhibits a liver disease in a standard dosage amount, and administering to the subject an RNF213 inhibitor; and
when the subject is heterozygous for an RNF213 predicted loss-of-function or missense variant, then administering or continuing to administer to the subject the therapeutic agent that treats or inhibits a liver disease in an amount that is the same as or lower than a standard dosage amount, and administering to the subject an RNF213 inhibitor;
wherein the presence of a genotype having the RNF213 predicted loss-of-function or missense variant nucleic acid molecule encoding the human RNF213 polypeptide indicates the subject has a reduced risk of developing a liver disease.

38. The method according to claim 37, wherein the subject is RNF213 reference, and the subject is administered or continued to be administered the therapeutic agent that treats or inhibits a liver disease in a standard dosage amount, and is administered an RNF213 inhibitor.

39. The method according to claim 37, wherein the subject is heterozygous for an RNF213 predicted loss-of-function or missense variant, and the subject is administered or continued to be administered the therapeutic agent that treats or inhibits a liver disease in an amount that is the same as or lower than a standard dosage amount, and is administered an RNF213 inhibitor.

40. The method according to any one of claims 37 to 39, wherein the RNF213 predicted loss-of-function or missense variant nucleic acid molecule is a nucleic acid molecule encoding Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu.

41. The method according to any one of claims 37 to 39, wherein the RNF213 predicted loss-of-function or missense variant nucleic acid molecule is a nucleic acid molecule encoding Glu3915Gly or Val3838Leu.

42. The method according to claim 40, wherein the RNF213 predicted loss-of-function or missense variant nucleic acid molecule is:
a genomic nucleic acid molecule having a nucleotide sequence comprising: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4;
an mRNA molecule having a nucleotide sequence comprising: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23; or
a cDNA molecule produced from an mRNA molecule, wherein the cDNA molecule has a nucleotide sequence comprising: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

43. The method according to any one of claims 37 to 42, wherein the genotyping assay comprises sequencing at least a portion of the nucleotide sequence of the RNF213 genomic nucleic acid molecule in the biological sample, wherein the sequenced portion comprises a position corresponding to: position 102,917 according to SEQ ID NO:2, or the complement thereof; position 102,391 according to SEQ ID NO:3, or the complement thereof; or position 103,226 according to SEQ ID NO:4, or the complement thereof;
wherein when the sequenced portion of the RNF213 genomic nucleic acid molecule in the biological sample comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4, then the RNF213 genomic nucleic acid molecule in the biological sample is an RNF213 predicted loss-of-function or missense variant genomic nucleic acid molecule.

44. The method according to any one of claims 37 to 42, wherein the genotyping assay comprises sequencing at least a portion of the nucleotide sequence of the RNF213 mRNA molecule in the biological sample, wherein the sequenced portion comprises a position corresponding to: position 11,887 according to SEQ ID NO:12, or the complement thereof;
position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof;
or position 84 according to SEQ ID NO:18, or the complement thereof; position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof;
or position 206 according to SEQ ID NO:23, or the complement thereof;
wherein when the sequenced portion of the RNF213 mRNA molecule in the biological sample comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23, then the RNF213 mRNA molecule in the biological sample is an RNF213 predicted loss-of-function or missense variant mRNA molecule.

45. The method according to any one of claims 37 to 42, wherein the genotyping assay comprises sequencing at least a portion of the nucleotide sequence of the RNF213 cDNA molecule in the biological sample, wherein the sequenced portion comprises a position corresponding to: position 11,887 according to SEQ ID NO:31, or the complement thereof;
position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof;
position 84 according to SEQ ID NO:37, or the complement thereof; position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof;
or position 206 according to SEQ ID NO:42, or the complement thereof;
wherein when the sequenced portion of the RNF213 cDNA molecule in the biological sample comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42, then the RNF213 cDNA molecule in the biological sample is an RNF213 predicted loss-of-function or missense variant cDNA molecule.

46. The method according to any one of claims 37 to 42, wherein the genotyping assay comprises:
a) contacting the biological sample with a primer hybridizing to a portion of the nucleotide sequence of the RNF213 genomic nucleic acid molecule that is proximate to a position corresponding to: position 102,917 according to SEQ ID NO:2, position 102,391 according to SEQ ID NO:3, or position 103,226 according to SEQ ID NO:4;
b) extending the primer at least through the position of the nucleotide sequence of the RNF213 genomic nucleic acid molecule corresponding to: position 102,917 according to SEQ ID NO:2, position 102,391 according to SEQ ID NO:3, or position 103,226 according to SEQ ID NO:4; and
c) determining whether the extension product of the primer comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4.

47. The method according to any one of claims 37 to 42, wherein the genotyping assay comprises:
a) contacting the biological sample with a primer hybridizing to a portion of the nucleotide sequence of the RNF213 mRNA molecule that is proximate to a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23;
b) extending the primer at least through the position of the nucleotide sequence of the RNF213 mRNA molecule corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and
c) determining whether the extension product of the primer comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23.

48. The method according to any one of claims 37 to 42, wherein the genotyping assay comprises:
a) contacting the biological sample with a primer hybridizing to a portion of the nucleotide sequence of the RNF213 cDNA molecule that is proximate to a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42;
b) extending the primer at least through the position of the nucleotide sequence of the RNF213 cDNA molecule corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42; and
c) determining whether the extension product of the primer comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

49. The method according to any one of claims 43 to 48, wherein the genotyping assay comprises sequencing the entire nucleic acid molecule.

50. The method according to any one of claims 37 to 42, wherein the genotyping assay comprises:
a) amplifying at least a portion of the genomic nucleic acid molecule that encodes the human RNF213 polypeptide, wherein the portion comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
d) detecting the detectable label.

51. The method according to any one of claims 37 to 42, wherein the genotyping assay comprises:
a) amplifying at least a portion of the mRNA molecule that encodes the human RNF213 polypeptide, wherein the portion comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and
d) detecting the detectable label.

52. The method according to any one of claims 37 to 42, wherein the genotyping assay comprises:
a) amplifying at least a portion of the cDNA molecule that encodes the human RNF213 polypeptide, wherein the portion comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
d) detecting the detectable label.

53. The method according to claim 52, wherein the nucleic acid molecule in the sample is mRNA and the mRNA is reverse-transcribed into cDNA prior to the amplifying step.

54. The method according to any one of claims 37 to 42, wherein the genotyping assay comprises:
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
detecting the detectable label.

55. The method according to any one of claims 37 to 42, wherein the genotyping assay comprises:
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23; and
detecting the detectable label.

56. The method according to any one of claims 37 to 42, wherein the genotyping assay comprises:
contacting the cDNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
detecting the detectable label.

57. The method according to any one of claims 37 to 56, wherein the nucleic acid molecule is present within a cell obtained from the subject.

58. The method according to any one of claims 37 to 57, wherein the RNF213 inhibitor comprises an antisense nucleic acid molecule, a small interfering RNA (siRNA), or a short hairpin RNA (shRNA) that hybridizes to an RNF213 mRNA.

59. The method according to any one of claims 37 to 57, wherein the RNF213 inhibitor comprises a Cas protein and guide RNA (gRNA) that hybridizes to a gRNA recognition sequence within an RNF213 genomic nucleic acid molecule.

60. The method according to claim 59, wherein the Cas protein is Cas9 or Cpf1.

61. The method according to claim 59 or claim 60, wherein the gRNA recognition sequence includes or is proximate to a position corresponding to: position 102,917 according to SEQ ID NO:1, position 102,391 according to SEQ ID NO:1, or position 103,226 according to SEQ ID NO:1.

62. The method according to claim 59 or claim 60, wherein the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 102,917 according to SEQ ID NO:1, position 102,391 according to SEQ ID NO:1, or position 103,226 according to SEQ ID NO:1.

63. The method according to claim 59 or claim 60, wherein a Protospacer Adjacent Motif (PAM) sequence is about 2 to 6 nucleotides downstream of the gRNA recognition sequence.

64. The method according to any one of claims 59 to 63, wherein the gRNA comprises from about 17 to about 23 nucleotides.

65. The method according to any one of claims 59 to 64, wherein the gRNA recognition sequence comprises a nucleotide sequence according to any one of SEQ ID NOs:62-81.

66. A method of identifying a subject having an increased risk for developing a liver disease, wherein the method comprises:
determining or having determined the presence or absence of a Ring Finger Protein 213 (RNF213) predicted loss-of-function or missense variant nucleic acid molecule encoding a human RNF213 polypeptide in a biological sample obtained from the subject;
wherein:
when the subject is RNF213 reference, then the subject has an increased risk for developing a liver disease; and
when the subject is heterozygous for an RNF213 predicted loss-of-function or missense variant or homozygous for an RNF213 predicted loss-of-function or missense variant, then the subject has a decreased risk for developing a liver disease.

67. The method according to claim 66, wherein the RNF213 predicted loss-of-function or missense variant nucleic acid molecule is a nucleic acid molecule encoding Glu3915Gly, Glu3964Gly, Glu822Gly, Glu350Gly, Glu146Gly, Glu37Gly, Glu28Gly, Val3838Leu, Val3887Leu, Val745Leu, Val273Leu, or Val69Leu.

68. The method according to claim 66, wherein the RNF213 predicted loss-of-function or missense variant nucleic acid molecule is a nucleic acid molecule encoding Glu3915Gly or Val3838Leu.

69. The method according to claim 67, wherein the RNF213 predicted loss-of-function or missense variant nucleic acid molecule is:
a genomic nucleic acid molecule having a nucleotide sequence comprising: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4;
an mRNA molecule having a nucleotide sequence comprising: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23; or
a cDNA molecule produced from an mRNA molecule, wherein the cDNA molecule has a nucleotide sequence comprising: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

70. The method according to any one of claims 66 to 69, wherein the determining step is carried out in vitro.

71. The method according to any one of claims 66 to 70, wherein the determining step comprises sequencing at least a portion of the nucleotide sequence of the RNF213 genomic nucleic acid molecule in the biological sample, wherein the sequenced portion comprises a position corresponding to: position 102,917 according to SEQ ID NO:2, or the complement thereof; position 102,391 according to SEQ ID NO:3, or the complement thereof; or position 103,226 according to SEQ ID NO:4, or the complement thereof;
wherein when the sequenced portion of the RNF213 genomic nucleic acid molecule in the biological sample comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4, then the RNF213 genomic nucleic acid molecule in the biological sample is an RNF213 predicted loss-of-function or missense variant genomic nucleic acid molecule.

72. The method according to any one of claims 66 to 70, wherein the determining step comprises sequencing at least a portion of the nucleotide sequence of the RNF213 mRNA molecule in the biological sample, wherein the sequenced portion comprises a position corresponding to: position 11,887 according to SEQ ID NO:12, or the complement thereof;
position 12,036 according to SEQ ID NO:13, or the complement thereof; position 2,685 according to SEQ ID NO:14, or the complement thereof; position 1,050 according to SEQ ID NO:15, or the complement thereof; position 438 according to SEQ ID NO:16, or the complement thereof; position 112 according to SEQ ID NO:17, or the complement thereof;
or position 84 according to SEQ ID NO:18, or the complement thereof; position 11,655 according to SEQ ID NO:19, or the complement thereof; position 11,804 according to SEQ ID NO:20, or the complement thereof; position 2,453 according to SEQ ID NO:21, or the complement thereof; position 818 according to SEQ ID NO:22, or the complement thereof;
or position 206 according to SEQ ID NO:23, or the complement thereof;
wherein when the sequenced portion of the RNF213 mRNA molecule in the biological sample comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23; then the RNF213 mRNA molecule in the biological sample is an RNF213 predicted loss-of-function or missense variant mRNA molecule.

73. The method according to any one of claims 66 to 70, wherein the determining step comprises sequencing at least a portion of the nucleotide sequence of the RNF213 cDNA molecule in the biological sample, wherein the sequenced portion comprises a position corresponding to: position 11,887 according to SEQ ID NO:31, or the complement thereof; position 12,036 according to SEQ ID NO:32, or the complement thereof; position 2,685 according to SEQ ID NO:33, or the complement thereof; position 1,050 according to SEQ ID NO:34, or the complement thereof; position 438 according to SEQ ID NO:35, or the complement thereof; position 112 according to SEQ ID NO:36, or the complement thereof;
position 84 according to SEQ ID NO:37, or the complement thereof; position 11,655 according to SEQ ID NO:38, or the complement thereof; position 11,804 according to SEQ ID NO:39, or the complement thereof; position 2,453 according to SEQ ID NO:40, or the complement thereof; position 818 according to SEQ ID NO:41, or the complement thereof; or position 206 according to SEQ ID NO:42, or the complement thereof;
wherein when the sequenced portion of the RNF213 cDNA molecule in the biological sample comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42; then the RNF213 cDNA molecule in the biological sample is an RNF213 predicted loss-of-function or missense variant cDNA molecule.

74. The method according to any one of claims 66 to 70, wherein the determining step comprises:
a) contacting the biological sample with a primer hybridizing to a portion of the nucleotide sequence of the RNF213 genomic nucleic acid molecule that is proximate to a position corresponding to: position 102,917 according to SEQ ID NO:2, position 102,391 according to SEQ ID NO:3, or position 103,226 according to SEQ ID NO:4;
b) extending the primer at least through the position of the nucleotide sequence of the RNF213 genomic nucleic acid molecule corresponding to: position 102,917 according to SEQ ID NO:2, position 102,391 according to SEQ ID NO:3, or position 103,226 according to SEQ ID NO:4; and
c) determining whether the extension product of the primer comprises: a guanine at a position corresponding to position 102,917 according to SEQ ID NO:2, a cytosine at a position corresponding to position 102,391 according to SEQ ID NO:3, or a thymine at a position corresponding to position 103,226 according to SEQ ID NO:4.

75. The method according to any one of claims 66 to 70, wherein the determining step comprises:
a) contacting the biological sample with a primer hybridizing to a portion of the nucleotide sequence of the RNF213 mRNA molecule that is proximate to a position corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23;
b) extending the primer at least through the position of the nucleotide sequence of the RNF213 mRNA molecule corresponding to: position 11,887 according to SEQ ID NO:12, position 12,036 according to SEQ ID NO:13, position 2,685 according to SEQ ID NO:14, position 1,050 according to SEQ ID NO:15, position 438 according to SEQ ID NO:16, position 112 according to SEQ ID NO:17, position 84 according to SEQ ID NO:18, position 11,655 according to SEQ ID NO:19, position 11,804 according to SEQ ID NO:20, position 2,453 according to SEQ ID NO:21, position 818 according to SEQ ID NO:22, or position 206 according to SEQ ID NO:23; and
c) determining whether the extension product of the primer comprises: a guanine at a position corresponding to position 11,887 according to SEQ ID NO:12, a guanine at a position corresponding to position 12,036 according to SEQ ID NO:13, a guanine at a position corresponding to position 2,685 according to SEQ ID NO:14, a guanine at a position corresponding to position 1,050 according to SEQ ID NO:15, a guanine at a position corresponding to position 438 according to SEQ ID NO:16, a guanine at a position corresponding to position 112 according to SEQ ID NO:17, a guanine at a position corresponding to position 84 according to SEQ ID NO:18, a cytosine at a position corresponding to position 11,655 according to SEQ ID NO:19, a cytosine at a position corresponding to position 11,804 according to SEQ ID NO:20, a cytosine at a position corresponding to position 2,453 according to SEQ ID NO:21, a cytosine at a position corresponding to position 818 according to SEQ ID NO:22, or a cytosine at a position corresponding to position 206 according to SEQ ID NO:23.

76. The method according to any one of claims 66 to 70, wherein the determining step comprises:
a) contacting the biological sample with a primer hybridizing to a portion of the nucleotide sequence of the RNF213 cDNA molecule that is proximate to a position corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, or position 206 according to SEQ ID NO:42;
b) extending the primer at least through the position of the nucleotide sequence of the RNF213 cDNA molecule corresponding to: position 11,887 according to SEQ ID NO:31, position 12,036 according to SEQ ID NO:32, position 2,685 according to SEQ ID NO:33, position 1,050 according to SEQ ID NO:34, position 438 according to SEQ ID NO:35, position 112 according to SEQ ID NO:36, position 84 according to SEQ ID NO:37, position 11,655 according to SEQ ID NO:38, position 11,804 according to SEQ ID NO:39, position 2,453 according to SEQ ID NO:40, position 818 according to SEQ ID NO:41, position 206 according to SEQ ID NO:42; and
c) determining whether the extension product of the primer comprises: a guanine at a position corresponding to position 11,887 according to SEQ ID NO:31, a guanine at a position corresponding to position 12,036 according to SEQ ID NO:32, a guanine at a position corresponding to position 2,685 according to SEQ ID NO:33, a guanine at a position corresponding to position 1,050 according to SEQ ID NO:34, a guanine at a position corresponding to position 438 according to SEQ ID NO:35, a guanine at a position corresponding to position 112 according to SEQ ID NO:36, a guanine at a position corresponding to position 84 according to SEQ ID NO:37, a cytosine at a position corresponding to position 11,655 according to SEQ ID NO:38, a cytosine at a position corresponding to position 11,804 according to SEQ ID NO:39, a cytosine at a position corresponding to position 2,453 according to SEQ ID NO:40, a cytosine at a position corresponding to position 818 according to SEQ ID NO:41, or a cytosine at a position corresponding to position 206 according to SEQ ID NO:42.

77. The method according to any one of claims 71 to 76, wherein the determining step comprises sequencing the entire nucleic acid molecule.

78. The method according to any one of claims 66 to 70, wherein the determining step comprises:
a) amplifying at least a portion of the genomic nucleic acid molecule that encodes the human RNF213 polypeptide, wherein the portion comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
d) detecting the detectable label.

79. The method according to any one of claims 66 to 70, wherein the determining step comprises:
a) amplifying at least a portion of the mRNA molecule that encodes the human RNF213 polypeptide, wherein the portion comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and
d) detecting the detectable label.

80. The method according to any one of claims 66 to 70, wherein the determining step comprises:
a) amplifying at least a portion of the cDNA molecule that encodes the human RNF213 polypeptide, wherein the portion comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
d) detecting the detectable label.

81. The method according to claim 80, wherein the nucleic acid molecule in the sample is mRNA and the mRNA is reverse-transcribed into cDNA prior to the amplifying step.

82. The method according to any one of claims 66 to 70, wherein the detecting step comprises:
contacting the genomic nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof; and
detecting the detectable label.

83. The method according to any one of claims 66 to 70, wherein the detecting step comprises:
contacting the mRNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; and
detecting the detectable label.

84. The method according to any one of claims 66 to 70, wherein the detecting step comprises:
contacting the cDNA molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof; and
detecting the detectable label.

85. The method according to any one of claims 66 to 84, wherein the subject is RNF213 reference, and the subject is administered a therapeutic agent that treats or inhibits a liver disease in a standard dosage amount, and is administered an RNF213 inhibitor.

86. The method according to any one of claims 66 to 84, wherein the subject is heterozygous for an RNF213 predicted loss-of-function or missense variant, and the subject is administered a therapeutic agent that treats or inhibits a liver disease in an amount that is the same as or lower than a standard dosage amount, and is administered an RNF213 inhibitor.

87. A therapeutic agent that treats or inhibits a liver disease for use in the treatment of a liver disease in a subject having:
a genomic nucleic acid molecule having a nucleotide sequence encoding a human Ring Finger Protein 213 (RNF213) polypeptide, wherein the nucleotide sequence comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof;
an mRNA molecule having a nucleotide sequence encoding a human RNF213 polypeptide, wherein the nucleotide sequence comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; or
a cDNA molecule having a nucleotide sequence encoding a human RNF213 polypeptide, wherein the nucleotide sequence comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof.

88. A Ring Finger Protein 213 (RNF213) inhibitor for use in the treatment of a liver disease in a subject having:
a genomic nucleic acid molecule having a nucleotide sequence encoding a human Ring Finger Protein 213 polypeptide, wherein the nucleotide sequence comprises: a guanine at a position corresponding to position 102,917 according to SEQ ID NO:2, or the complement thereof; a cytosine at a position corresponding to position 102,391 according to SEQ ID NO:3, or the complement thereof; or a thymine at a position corresponding to position 103,226 according to SEQ ID NO:4, or the complement thereof;
an mRNA molecule having a nucleotide sequence encoding a human RNF213 polypeptide, wherein the nucleotide sequence comprises: a guanine at a position corresponding to position 11,887 according to SEQ ID NO:12, or the complement thereof; a guanine at a position corresponding to position 12,036 according to SEQ ID NO:13, or the complement thereof; a guanine at a position corresponding to position 2,685 according to SEQ ID NO:14, or the complement thereof; a guanine at a position corresponding to position 1,050 according to SEQ ID NO:15, or the complement thereof; a guanine at a position corresponding to position 438 according to SEQ ID NO:16, or the complement thereof; a guanine at a position corresponding to position 112 according to SEQ ID NO:17, or the complement thereof; a guanine at a position corresponding to position 84 according to SEQ ID NO:18, or the complement thereof; a cytosine at a position corresponding to position 11,655 according to SEQ ID NO:19, or the complement thereof; a cytosine at a position corresponding to position 11,804 according to SEQ ID NO:20, or the complement thereof; a cytosine at a position corresponding to position 2,453 according to SEQ ID NO:21, or the complement thereof; a cytosine at a position corresponding to position 818 according to SEQ ID NO:22, or the complement thereof; or a cytosine at a position corresponding to position 206 according to SEQ ID NO:23, or the complement thereof; or
a cDNA molecule having a nucleotide sequence encoding a human RNF213 polypeptide, wherein the nucleotide sequence comprises: a guanine at a position corresponding to position 11,887 according to SEQ ID NO:31, or the complement thereof; a guanine at a position corresponding to position 12,036 according to SEQ ID NO:32, or the complement thereof; a guanine at a position corresponding to position 2,685 according to SEQ ID NO:33, or the complement thereof; a guanine at a position corresponding to position 1,050 according to SEQ ID NO:34, or the complement thereof; a guanine at a position corresponding to position 438 according to SEQ ID NO:35, or the complement thereof; a guanine at a position corresponding to position 112 according to SEQ ID NO:36, or the complement thereof; a guanine at a position corresponding to position 84 according to SEQ ID NO:37, or the complement thereof; a cytosine at a position corresponding to position 11,655 according to SEQ ID NO:38, or the complement thereof; a cytosine at a position corresponding to position 11,804 according to SEQ ID NO:39, or the complement thereof; a cytosine at a position corresponding to position 2,453 according to SEQ ID NO:40, or the complement thereof; a cytosine at a position corresponding to position 818 according to SEQ ID NO:41, or the complement thereof; or a cytosine at a position corresponding to position 206 according to SEQ ID NO:42, or the complement thereof.

89. The RNF213 inhibitor according to claim 88, which is an antisense nucleic acid molecule, a small interfering RNA (siRNA), or a short hairpin RNA (shRNA) that hybridizes to an RNF213 mRNA.

90. The RNF213 inhibitor according to claim 88, which comprises a Cas protein and guide RNA (gRNA) that hybridizes to a gRNA recognition sequence within an RNF213 genomic nucleic acid molecule.

91. The RNF213 inhibitor according to claim 90, wherein the Cas protein is Cas9 or Cpf1.

92. The RNF213 inhibitor according to claim 90 or claim 91, wherein the gRNA recognition sequence includes or is proximate to: position 102,917 according to SEQ ID NO:1, position 102,391 according to SEQ ID NO:1, or position 103,226 according to SEQ ID NO:1.

93. The RNF213 inhibitor according to claim 90 or claim 91, wherein the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 102,917 according to SEQ ID NO:1, position 102,391 according to SEQ ID NO:1, or position 103,226 according to SEQ ID NO:1.

94. The RNF213 inhibitor according to claim 90 or claim 91, wherein a Protospacer Adjacent Motif (PAM) sequence is about 2 to about 6 nucleotides downstream of the gRNA recognition sequence.

95. The RNF213 inhibitor according to any one of claims 90 to 94, wherein the gRNA comprises from about 17 to about 23 nucleotides.

96. The RNF213 inhibitor according to any one of claims 90 to 95, wherein the gRNA recognition sequence comprises a nucleotide sequence according to any one of SEQ ID NOs:62-81.
